(19) United States
(12) Patent Application Publication
(10) Pub. No.: US 2021/0261966 A1
(43) Pub. Date: Aug. 26, 2021

(54) TREATMENT OF CARDIOMYOPATHY THROUGH MODULATION OF HYPOXIA-INDUCED eRNA ACTIVITY

(71) Applicant: UNITED KINGDOM RESEARCH AND INNOVATION, Swindon (GB)

(72) Inventor: Jaya KRISHNAN, Frankfurt am Main Hessen (DE)

(73) Assignee: UNITED KINGDOM RESEARCH AND INNOVATION, Swindon (GB)

(21) Appl. No.: 17/256,293

(22) PCT Filed: Jun. 28, 2019
(86) PCT No.: PCT/EP2019/067494

§ 371 Date: Dec. 28, 2020

(30) Foreign Application Priority Data
Jun. 28, 2018 (EP) 18180595.3

(51) Int. Cl. C12N 15/113; A61K 31/7125; A61K 47/69; A61K 35/76; A61P 9/04

(52) U.S. Cl. CPC C12N 15/1135; A61K 31/7125; A61K 47/6949; A61K 35/76; C12N 2310/321; C12N 2310/11; C12N 2310/3231; C12N 2310/3513; C12N 2310/315; A61P 9/04

(57) ABSTRACT

The invention relates to an inhibitor, in particular an antisense oligonucleotide, directed against the enhancer RNA SINT1 for use in a method for the prevention or treatment of heart disease, particularly for cardiomyopathy. The antisense oligonucleotide is a gapmer or RNA interference (RNAi) agent. The antisense oligonucleotide is particularly suitable for the treatment of cardiomyopathy resulting from cardiac overload.

The present invention relates to inhibitors targeting stress induced non-coding RNA transcript 1 (SINT1).

### BACKGROUND OF THE INVENTION

Chronic heart failure is characterized by clinical symptoms of cardiac dysfunction and represents the culmination of prolonged left ventricular growth in response to pathologic stressors including ischemia, hypertension, aortic stenosis, and genetic mutations. Although it is a leading cause of hospitalization and mortality worldwide, and in spite of its immense impact on human health and the associated socio-economic implications, therapeutic strategies for chronic heart failure remain limited. Of particular concern is the fact that current treatment modalities (based on heart glycosides, diuretics, vasodilatators or neurohumoral intervention) have limited potential to treat the underlying cause of heart disease. Thus, its prevalence, morbidity and mortality remain high, and with increasing hospitalization and health care costs, heart failure threatens to become a pandemic health issue. Hence, drug discovery should ideally focus on targeting key causative molecular drivers of disease maintenance and progression.

The advent of genomewide transcriptomic and epigenetic analysis has not only deepened the mechanistic understanding of gene regulation but has also served to unveil a novel class of gene modulating non-coding RNAs, termed enhancer-templated RNAs (eRNAs). Enhancers are regulatory DNA elements that bind transcription factors to induce gene transcription through the formation of secondary structures that mediate the interaction of the enhancer with the promoter. Transcription at enhancer elements positively correlates with enhancer activity and is characterised by high histone 3 lysine 4 monomethylation (H3K4me1) and low or absent H3K4 trimethylation (me3). In addition to H3K4 methylation marks, enrichment of H3K27 acetylation and absence of the repressive H3K27me3 signature are other characteristics of active enhancers and correlate positively with the expression of eRNAs.

eRNA transcription has been shown to be regulated by specific transcription factors and their expression serves to induce transcription of either one or both neighbouring 5′ and 3′ genes (De Santa et al., PLoS Biol 8, e1000384, 2010; Li et al., Nature 498, 516, 2013). A number of eRNAs have been identified in the heart but their biological function remains unclear (Ounzain et al., Eur Heart J 36, 353, 2015). Active enhancers during heart development have been identified by genomewide chromatin immunoprecipitation with antibodies against the transcriptional coactivator p300 coupled to parallel sequencing (ChIP-Seq) on mouse embryonic hearts. A report by Ounzain et al. revealed that a subset of active enhancer loci produces eRNAs and that the majority of these transcripts are selectively expressed in the heart. The eRNAs mm67 and mm85 identified by Ounzain et al. positively correlated with the expression of the flanking coding gene myocardin. Recent studies have also identified eRNAs induced by myocardial infarction and transaortic constriction surgery (TAC) in mice (Ounzain et al., J Mol Cell Cardiol 76, 55, 2014). eRNA mm132 expression was shown to be elevated in response to both stressors and correlated positively with the upregulation of its flanking gene endothelin 1, which has been implicated in cardiac hypertrophy and heart failure. Furthermore, eRNA Novlnc6 expression is inhibited in human patients with dilated cardiomyopathy and in a mouse model of myocardial infarction. Thus, the specific modulation of cell-type and stress-responsive eRNAs has the potential to very precisely influence pathophysiologic gene networks.

Hypoxia inducible factors (HIFs) are heterodimeric transcription factors composed of HIF1α and HIF1β subunits that occupy central roles in regulating oxygen homeostasis (Wang et al., Proc Natl Acad Sci USA 92, 5510, 1995) and the pathogenesis of human disease including cancer and cardiovascular disease (Semenza, Cell 148, 399, 2012). They are activated in hypoxic tissue to induce a transcriptional program embracing coding and non-coding RNA transcripts that are entrusted to modulate both the supply and consumption of oxygen. To date however, it is unclear if HIFs also activate transcription of eRNAs to afford cell- and signal-specificity of select HIF output responses.

In the present invention a conserved pathology-induced eRNA, termed stress induced non-coding RNA transcript 1 (SINT1), was identified as a critical determinant of maladaptive cardiac growth, disease-associated metabolic reprogramming and contractile dysfunction. SINT1 expression is restricted to the ventricular myocardium and imposes function through induction of a pro-hypertrophic gene cluster comprising of suppressor of morphogenesis in genitalia 1 (SMG1) and synaptotagmin XVII (SYT17). SINT1 induction correlates with hypertrophic cardiomyopathy in humans and mice, and in vivo inactivation, in particular anti sense oligonucleotide (ASO)-mediated inactivation, of SINT1 surprisingly prevents stress-induced cardiac pathogenesis and reverses pathology and dramatically improves overall survival in diseased mice. Mechanistically, the inventors disclose SINT1 interaction at the promoters of SMG1 and SYT17 and uncouple their impact on gene programs critical for the development of pathologic cardiac hypertrophy and its progression to heart failure.

Based on the above-mentioned state of the art, the objective of the present invention is to provide means and methods for treatment of cardiomyopathy. This objective is attained by the subject matter of the claims of the present specification.

### DESCRIPTION

The term inhibitor in the context of the present specification relates to oligonucleotide agents that bind specifically to either the transcribed enhancer RNA SINT1 or the genomic region encoding SINT1, thereby decreasing or abolishing the molecular function of SINT1.

“Capable of forming a hybrid” in the context of the present invention relates to sequences that under the conditions existing within the cytosol of a mammalian cell, are able to bind selectively to their target sequence. Such hybridizing sequences may be contiguously reverse-complimentary to the target sequence, or may comprise gaps, mismatches or additional non-matching nucleotides. The minimal length for a sequence to be capable of forming a hybrid depends on its composition, with C or G nucleotides contributing more to the energy of binding than A or T/U nucleotides, and the backbone chemistry.

The term oligonucleic acid agent in the context of the present specification refers to an oligonucleotide capable of specifically binding to and leading to a significant reduction of the physiological role of SINT1. Examples of oligonucleic acid agents of the present invention are antisense oligomers made of DNA, DNA having phosphorothioate modified linkages in their backbone, ribonucleotide oligomers, RNA comprising bridged or locked nucleotides, particularly wherein the ribose ring is connected by a methylene bridge between the 2′-O and 4′-C atoms, RNA having phosphorothioate modified linkages in their backbone or any mixture of deoxyribonucleotide and ribonucleotide bases as an oligomer.

The term antisense oligonucleotide or oligonucleotide agent in the context of the present specification refers to any oligonucleotide capable of specifically binding to and leading to a significant reduction of the physiological role of SINT1. Examples of antisense oligonucleotides of the present invention are antisense oligomers made of DNA, DNA having phosphorothioate modified linkages in their backbone, ribonucleotide oligomers, RNA comprising bridged or locked nucleotides, particularly wherein the ribose ring is connected by a methylene bridge between the 2′-O and 4′-C atoms, RNA having phosphorothioate modified linkages in their backbone or any mixture of deoxyribonucleotide and ribonucleotide bases as an oligomer. The terms oligonucleic acid agent and antisense oligonucleotide or oligonucleotide agent are used interchangeably in the present specification.

In certain embodiments, the antisense oligonucleotide of the invention comprises analogues of nucleic acids such as phosphotioates, 2′O-methylphosphothioates, peptide nucleic acids (PNA; N-(2-aminoethyl)-glycine units linked by peptide linkage, with the nucleobase attached to the alpha-carbon of the glycine) or locked nucleic acids (LNA; 2′O, 4′C methylene bridged RNA building blocks). The antisense sequence may be composed partially of any of the above analogues of nucleic acids, with the rest of the nucleotides being “native” ribonucleotides occurring in nature, or may be mixtures of different analogues, or may be entirely composed of one kind of analogue.

The term gapmer is used in its meaning known in the field of molecular biology and refers to an antisense oligonucleotide complementary to its target sequence, that comprises a central block of a deoxyribonucleotide oligomer flanked by short ribonucleotide oligomers. The flanking ribonucleotide oligomers consist of nuclease and protease resistant ribonucleotides. In certain embodiments, the nuclease and protease resistant ribonucleotides comprise 2′-O modified ribonucleotides, in particular bridged nucleic acids with a bridge between the 2′-O and 4′-C of the ribose moiety.

“Nucleotides” in the context of the present invention are nucleic acid or nucleic acid analogue building blocks, oligomers of which are capable of forming selective hybrids with miRNA oligomers on the basis of base pairing. The term nucleotides in this context includes the classic ribonucleotide building blocks adenosine, guanosine, uridine (and ribosylthymin), cytidine, the classic deoxyribonucleotides deoxyadenosine, deoxyguanosine, thymidine, deoxyuridine and deoxycytidine. It further includes analogues of nucleic acids such as phosphotioates, 2′O-methylphosphothioates, peptide nucleic acids (PNA; N-(2-aminoethyl)-glycine units linked by peptide linkage, with the nucleobase attached to the alpha-carbon of the glycine) or locked nucleic acids (LNA; 2′O, 4′C methylene bridged RNA building blocks). The hybridizing sequence may be composed of any of the above nucleotides, or mixtures thereof.

In the context of the present specification, the terms sequence identity and percentage of sequence identity refer to the values determined by comparing two aligned sequences. Methods for alignment of sequences for comparison are well-known in the art. Alignment of sequences for comparison may be conducted by the local homology algorithm of Smith and Waterman, Adv. Appl. Math. 2:482 (1981), by the global alignment algorithm of Needleman and Wunsch, J. Mol. Biol. 48:443 (1970), by the search for similarity method of Pearson and Lipman, Proc. Nat. Acad. Sci. 85:2444 (1988) or by computerized implementations of these algorithms, including, but not limited to: CLUSTAL, GAP, BESTFIT, BLAST, FASTA and TFASTA. Software for performing BLAST analyses is publicly available, e.g., through the National Center for Biotechnology-Information (http://blast.ncbi.nlm.nih.gov/).

One example for comparison of amino acid sequences is the BLASTP algorithm that uses the default settings: Expect threshold: 10; Word size: 3; Max matches in a query range: 0; Matrix: BLOSUM62; Gap Costs: Existence 11, Extension 1; Compositional adjustments: Conditional compositional score matrix adjustment. One such example for comparison of nucleic acid sequences is the BLASTN algorithm that uses the default settings: Expect threshold: 10; Word size: 28; Max matches in a query range: 0; Match/Mismatch Scores: 1.-2; Gap costs: Linear. Unless otherwise stated, sequence identity values provided herein refer to the value obtained using the BLAST suite of programs (Altschul et al., J. Mol. Biol. 215:403-410 (1990)) using the above identified default parameters for protein and nucleic acid comparison, respectively.

### DETAILED DESCRIPTION OF THE INVENTION

A first aspect of the invention relates to an inhibitor directed against the enhancer RNA SINT1 (SEQ ID NO 001), for use in a method for the prevention or treatment of heart disease, particularly cardiomyopathy. SEQ ID NO 001 represents the genomic template of the transcribed human SINT1 eRNA.

An alternative of this aspect relates to an oligonucleic acid agent directed at and capable of specifically inhibiting and/or degrading the enhancer RNA SINT1, for use in a method for the prevention or treatment of heart disease, particularly cardiomyopathy.

In certain embodiments, the oligonucleic acid agent comprises a sequence hybridizing to SINT1. The agent sequence is at least 95% identical, particularly 96%, 97%, 98%, 99% or 100% identical to a sequence selected from Table 3 or Table 4. In certain embodiments, the hybridizing sequence comprises deoxynucleotides, phosphothioate deoxynucleotides, LNA and/or PNA nucleotides or mixtures thereof.

In certain embodiments, the inhibitor or oligonucleic acid agent has a dissociation constant (K_{D}) smaller than 100 nM, in particular smaller than 50 nM, more particular smaller than 10 nM, in relation to its binding to the target SINT1.

In certain embodiments, the interaction of the inhibitor with other non-specifically bound targets has a KD larger than 1 μM, in particular larger than 10 μM, more particular larger than 100 μM.

In certain embodiments, the oligonucleic acid agent of the invention is an antisense oligonucleotide, particularly an antisense gapmer.

In certain embodiments, the inhibitor is an antisense oligonucleotide.

The oligonucleic acid agent of the invention is not only suitable for the prevention of cardiomyopathy, but is also able to reverse pathogenic alterations already established.

Antisense Composed Partially or Entirely of Nucleoside Analogues

In certain embodiments, the oligonucleic acid agent comprises or is essentially composed of LNA moieties and comprises about 20 or fewer nucleotides.

In certain embodiments, the oligonucleic acid agent is essentially composed of LNA moieties and is described by a sequence selected from Table 3. In a particular embodiment, the nucleoside analogues of any sequence of Table 3 are linked by phosphate esters. In a particular embodiment, the nucleoside analogues of any sequence of Table 3 are linked by phosphothioate esters.

In certain embodiments, the oligonucleic acid agent for use in a method of treatment or prevention of heart disease comprises, or essentially consists of one or several peptide nucleic acid (PNA) moieties.

In certain embodiments, the antisense oligonucleotides of the invention are between 8 and 40 bases in length, in particular between 12 and 16 bases in length, more particular between 15 and 16 bases in length.

In certain embodiments, the antisense oligonucleotide comprises ribonucleotides and deoxyribonucleotides, in particular modified ribonucleotides and modified deoxyribonucleotides. A non-limiting example of a modification of deoxyribonucleotides and ribonucleotides are phosphorothioate modified linkages in their backbone. A non-limiting example of a modification of ribonucleotides is a 2′-O to 4′-C bridge.

Antisense Gapmers

In certain embodiments, the oligonucleic acid agent is a gapmer characterized by a central DNA block, the sequence of which is complementary to SINT1, and which is flanked on either side (5′ and 3′) by nuclease-resistant LNA sequences which are also complementary to SINT1. The central DNA block contains the RNase H activating domain, in other words is the part that lead the target DNA to be hydrolyzed. In certain embodiments, the flanking LNA is fully phosphorothioated.

The flanking exonuclease-protected nucleoside analogues impart high binding energy. In certain embodiments, the flanking exonuclease-protected nucleoside analogues are characterized by a ribose unit having a 2′-O/4′-C bridge.

In certain embodiment the 2′-O/4′-C bridge of the flanking region of the gapmer is a five-membered, six-membered or seven-membered bridged structure.

In certain embodiments, the central deoxyribonucleotide oligomer block of the gapmer comprises at least 5 deoxyribonucleotides.

In certain embodiments, the oligonucleic acid agent comprises 12-20 nucleotides. In certain particular embodiments, the oligonucleic acid agent comprises 14-16 nucleotides.

In certain embodiments, the hybridizing sequence of the oligonucleic acid agent according to the invention comprises 14, 15 or 16 nucleotides.

In certain embodiments, the central deoxyribonucleotide oligomer block of the gapmer comprises a phosphate backbone between the deoxyribonucleotides.

In certain embodiments, the oligonucleic acid agent comprises, or essentially consists of, a central block of 5 to 10 deoxyribonucleotides linked by phosphate ester bonds flanked on either side by 2′-O modified ribonucleotides or PNA oligomers. In certain embodiments, the oligonucleic acid agent comprises, or essentially consists of, a central block of 5 to 10 deoxyribonucleosides flanked by LNA nucleoside analogues. In certain particular embodiments, said LNA nucleoside analogues are linked by phosphothioate moieties.

In certain embodiments, the oligonucleic acid agent of the invention comprises or essentially consists of any of the sequences of Table 4, wherein the non-underscore letters signify nucleoside analogues, particularly LNA, more particularly LNA linked by phosphothioate esters, and the central underscored letters signify DNA nucleosides linked by phosphate esters, and the link between a nucleoside analogue and a DNA nucleoside is selected from phosphate ester and thiophosphate.

RNAi/siRNA/shRNA

In certain embodiments, the oligonucleic acid agent of the invention is an RNA interference agent.

An RNA interference (RNAi) agent in the context of the present specification refers to a ribonucleotide oligomer that causes the degradation of its enhancer RNA (eRNA) target sequence.

In certain embodiments, the RNAi agents of the invention comprise, or consist of,
a single-stranded or double-stranded interfering ribonucleic acid oligomer or precursor thereof, comprising a sequence tract complementary to the targeted enhancer RNA molecule; or
a single-stranded or double-stranded antisense ribonucleic or deoxyribonucleic acid, comprising a sequence tract complementary to the targeted enhancer RNA molecule.

In certain embodiments, the sequence tract complementary to the targeted enhancer RNA molecule is a contiguous sequence tract 7, 8, 9, 10, 11, 12, 13, 14, 15, 16, 17, 18, 19, 20, 21 or 22 nucleotides in length.

In certain embodiments, the RNAi agents of the invention include, but are not limited to, small interfering RNAs (siRNAs), short hairpin RNAs (shRNAs), microRNAs and non-coding RNAs or the like, Morpholinos (phosphodiamidate morpholino oligomers) and Dicer substrate siRNAs (DsiRNAs, DsiRNAs are cleaved by the RNAse III class endoribonuclease Dicer into 21-23 base duplexes having 2-base 3′-overhangs), UsiRNAs (UsiRNAs are duplex siRNAs that are modified with non-nucleotide acyclic monomers, termed unlocked nucleobase analogues (UNA), where the bond between two adjacent carbon atoms of ribose is removed), self-delivering RNAs (sdRNAs) including rxRNA™ (RXi Pharmaceuticals, Westborough, Mass., USA).

In some embodiments, the RNAi agents of the invention comprise analogues of nucleic acids such as phosphotioates, 2′O-methylphosphothioates, peptide nucleic acids (PNA; N-(2-aminoethyl)-glycine units linked by peptide linkage, with the nucleobase attached to the alpha-carbon of the glycine) or locked nucleic acids (LNA; 2′O, 4′C methylene bridged RNA building blocks). The hybridizing sequence may be composed partially of any of the above nucleotides, with the rest of the nucleotides being “native” ribonucleotides occurring in nature, or may be mixtures of different analogues, or may be entirely composed of one kind of analogue.

In certain embodiments, the antisense oligonucleotide comprises or consist of the sequence of SEQ ID NO 060 to SEQ ID NO 202, for use in a method for the prevention or treatment of cardiomyopathy. In certain embodiments, the sequences of SEQ ID NO 060 to SEQ ID NO 202 contain modified nucleotides. In certain embodiments, the SEQ ID NO 060 to SEQ ID NO 202 are the corresponding RNA sequences (T to U) and are used as RNA interference agent.

In certain embodiments, the antisense oligonucleotide comprises or consist of the sequence of SEQ ID NO 060 to SEQ ID NO 069, for use in a method for the prevention or treatment of cardiomyopathy. In certain embodiments, the sequences of SEQ ID NO 060 to SEQ ID NO 069 contain modified nucleotides. In certain embodiments, the SEQ ID NO 060 to SEQ ID NO 069 are the corresponding RNA sequences (T to U) and are used as RNA interference agent.

In certain embodiments, the antisense oligonucleotide is for use in the treatment of cardiac hypertrophy.

In certain embodiments, the antisense oligonucleotide is for use in the treatment of a cardiomyopathy resulting of cardiac overload. Cardiac overload can result from a cardiac pressure overload or a cardiac volume overload. Non-limiting examples of cardiomyopathies resulting from cardiac overload are hypertension-induced pathologic hypertrophy, stenosis(blockage)-induced pathologic hypertrophy, hypertrophic cardiomyopathy (congenital and idiopathic), restrictive pathologic hypertrophy and ischemic heart disease.

In certain embodiments, the antisense oligonucleotide is for use in the treatment of hypertension-induced pathologic hypertrophy, stenosis(blockage)-induced pathologic hypertrophy, hypertrophic cardiomyopathy (congenital and idiopathic), restrictive pathologic hypertrophy or ischemic heart disease.

In some embodiments, the antisense oligonucleotide comprises deoxynucleotides, ribonucleotides, phosphothioate and/or 2′-O-methyl-modified phosphothioate ribonucleotides.

In some embodiments, the hybridizing sequence comprises deoxynucleotides, phosphothioate deoxynucleotides, phosphothioate ribonucleotides and/or 2′-O-methyl-modified phosphothioate ribonucleotides.

Another aspect of the invention relates to an antisense oligonucleotide directed against the enhancer RNA SINT1 (SEQ ID No 001) that comprises or consists of any one of the sequences of SEQ ID NO 060 to SEQ ID NO 202. In certain embodiments, the antisense oligonucleotide directed against the enhancer RNA SINT1 (SEQ ID No 001) comprises or consists of any one of the sequences of SEQ ID NO 060 to SEQ ID NO 069. In certain embodiments, the SEQ ID NO 060 to SEQ ID NO 202 are the corresponding RNA sequences (T to U) and are used as RNA interference agent. In certain embodiments, the SEQ ID NO 060 to SEQ ID NO 069 are the corresponding RNA sequences (T to U) and are used as RNA interference agent.

In certain embodiments, the oligonucleic agent of the invention is conjugated to, or encapsulated by, a nanoparticle, a virus and a lipid complex.

The invention is further illustrated by the following examples and figures, from which further embodiments and advantages can be drawn. These examples are meant to illustrate the invention but not to limit its scope.

### BRIEF DESCRIPTION OF THE FIGURES

FIG. 1 shows SINT1 identification and functional relevance. (A) UCSC Genome Browser (mm9 assembly) presentation of H3K4me1 and H3K4me3 modifications at the SINT1 genomic locus in sham, TAC, Vhl f/f and Vhl cKO cardiac left ventricle and RNA transcript generation. (B) Conservation of the SINT1 loci and its flanking regions across species. (C) Samples described in (A) as well as left ventricular samples from 1-Kidney/1-Clip experiments were subjected to ChIP with antibodies against total H3 and H3K27ac. H3K27ac abundance within the indicated genomic regions was calculated as the ratio between H3K27ac and total H3 (n=3 biological replicates; shown is mean±SD; *p<0.05; two-tailed unpaired t-test). (D) Vhl f/f and Vhl cKO left ventricular biopsies were assessed for SINT1, Smg1 and Syt17 RNA, normalized to Hprt1 mRNA (n=3 biological replicates; shown is mean±SEM; **p<0.01; one-way ANOVA followed by a Dunnet's multiple comparison post-test). (E) Vhl f/f and Vhl cKO left ventricular biopsies were assessed for Hif1α, Smg1 and Syt17 protein expression (normalized to sarcomeric α-actinin). (F) Hif1α f/f and Hif1α cKO mice subjected to TAC were assessed for SINT1, Smg1 and Syt17 RNA expression, normalized to Hprt (n=3 biological replicates; shown is mean±SEM; *p<0.05; one-way ANOVA followed by a Dunnet's multiple comparison post-test). (G) Hif1α f/f and Hif1α cKO mice subjected to TAC were assessed for Hif1α, Smg1 and Syt17 protein expression in cardiac left ventricle (normalized to sarcomeric α-actinin).

FIG. 2 shows that SINT1 is a HIF1α-dependent non-coding RNA with enhancer function. (A) Sequence of the human and mouse SINT1 promoter. Conserved HRE is shown in bold, with the core HRE motif capitalized. (B) NMC cultured at 20% O2 or 3% O2 and assessed for chromatin immunoprecipitation of the SINT1 promoter with a HIF1α-specific antibody (IP: HIF1a) or with a control isotype-matched antibody (IP: IgG control). (C) Hif1α-dependent SINT1 promoter activity was determined by transient transfection of wildtype or HRE-mutated SINT1 promoter, respectively, fused to luciferase and with either an empty vector control, HIF1αΔODD or PE stimulation, and co-transfection with a β-galactosidase construct for normalization of luciferase signal. (n=4 biological replicates; shown is mean±SD; *p<0.05; one-way ANOVA followed by a Dunnet's multiple comparison post-test). (D) 3 kb of the genomic region flanking the SINT1 transcript was assessed for enhancer activity in response to HIF1α by transient transfection of SINT1 fused to SV-40-luciferase, and transfection with an empty vector control or HIF1αΔODD at increasing concentrations, and co-transfection with a β-galactosidase construct for normalization of luciferase signal. The Pparγ serves as control. (n=4 biological replicates; shown is mean±SD; *p<0.05 two-tailed unpaired t-test compared to empty-vector control). (E) Schematic diagram of the SINT1-BoxB-AN tethering system. (F) Egr1 mRNA expression was assessed by qPCR after co-transfection of SINT1-BoxB with the AN-SRF fusion construct in 293T cells. Data was normalized to Hprt (n=3 biological replicates; shown is mean±SD; *p<0.05; one-way ANOVA followed by a Dunnet's multiple comparison post-test compared to 293T cells expressing BoxB). (G) NMC cultured as shown were assessed for Gapdh, Neat1 and SINT1 RNA by RT-qPCR and normalized to Hprt (n=3 biological replicates; shown is mean±SD; *p<0.05; two-tailed unpaired t-test compared to NMC treated in 20% O₂). (H) SINT1 interaction at the Smg1 and Syt17 promoter in hypoxia and normoxia was assessed by ChIRP-qPCR using promoter-specific primers. Gapdh and Neat1 promoter qPCR serve as controls (n=3 biological replicates; shown is mean±SD; *p<0.05; one-way ANOVA followed by a Dunnet's multiple comparison post-test). (I) −1.5 kb of the Smg1 (upper panel) and Syt17 (lower panel) promoter was divided into three domains (−1500 to −1000 bp, −1000 to −500 bp and −500 to 0 bp) and assessed by qPCR for SINT1 interaction. ChIRP pull-down of Gapdh and Neat1 RNA serve as controls. (n=3 biological replicates; shown is mean±SD; *p<0.05; one-way ANOVA followed by a Dunnet's multiple comparison post-test). (J) Representative images of SINT1, Smg1 and Syt17 RNA localization under normoxia and hypoxia as determined by RNA-FISH. Nuclei were stained with DAPI. Scale bar is 100 μm.

FIG. 3 shows that SINT1 expression levels affect expression of Smg1 and Syt17 as well as cellular growth and contractility. (A) NMC were subjected to 3% O2 were transduced with ns or two unique shRNAs targeting SINT1 and assessed for SINT1, Smg1 and Syt17 RNA, normalized to Hprt (n=3 biological replicates; shown is mean±SD; *p<0.05; one-way ANOVA followed by a Dunnet's multiple comparison post-test). (B) NMC cultured in 20% O2 or 3% O₂ in the presence of non-silencing shRNA (ns) or shRNA targeting SINT1 were assessed for Hif1α, Smg1, Upf1 and phospho-Upf1 and Syt17 protein levels. Loading is normalized to sarcomeric α-actinin. (C) Cardiomyocytes stimulated with ectopic HIF1αΔODD in combination with a control ns or two unique shRNAs targeting SINT1 were assessed for [³H]leucine incorporation. An empty vector treatment was used to control HIF1αΔODD treatment. n=3 biological replicates; shown is mean±SD; *p<0.05; two-tailed unpaired t-test compared to ns transduced (D, E) NMC transduced with empty vector or HIF1αΔODD. OCR and ECAR were assessed at the baseline state, following an injection of oleic acid (OA) and then Carbonyl cyanide-4-(trifluoromethoxy)phenylhydrazone (FCCP). Depicted are rates expressed as OCR to ECAR ratios (E), and quantified ECAR and OCR values (E) (n=8 biological replicates; shown is mean±SEM; *p<0.05; two-tailed unpaired t-test). FAO and MRC indicate fatty acid oxidation rate and mitochondrial respiratory complex capacity, respectively. (F,G) NMC transduced with ns or shSINT1 and empty vector (mock) or HIF1αΔODD lentiviruses. OCR and ECAR were assessed at the baseline state, following an injection of OA and then FCCP. Depicted are rates expressed as OCR to ECAR ratios (F), and quantified ECAR and OCR values (G) (n=8 biological replicates; shown is mean±SEM; *p<0.05; two-tailed unpaired t-test). FAO and MRC indicate fatty acid oxidation rate and mitochondrial respiratory complex capacity, respectively. (H) NMC treated as in F were stimulated at 1 Hz and assessed for contractile amplitude by IonOptix Contractility Recording analysis contractile amplitude quantified (H). Mock-transduced samples serve as controls. (n=3 biological replicates; shown is mean±SD; *, %, p<0.05; one-way ANOVA followed by a Dunnet's multiple comparison post-test). (I, J) Relative maximal velocity of contraction (I) and relaxation (J) were assessed in NMC expressing HIF1αΔODD in combination with ns or shSINT1 using the IonOptix contractility analyzer (n=3 biological replicates; shown is mean±SD; p<0.05; one-way ANOVA followed by a Dunnet's multiple comparison post-test).

FIG. 4 shows that Smg1 and Syt17 expression affects cardiomyocyte growth under pathologic stress whereas contractility is mainly regulated by Syt17. (A) NMCs were stained for cardiomyocyte-specific α-actinin, Smg1 and DAPI, and imaged by confocal microscopy. Representative fields are shown. Scale bar is 25 μm. (B) NMCs were stained for the mitochondrial marker Atp5a1, Syt17 and DAPI, and imaged by confocal microscopy. Representative fields are shown. Scale bar is 25 μm. (C,D) NMC cultured in 20% O2 or 3% O2 in the presence of non-silencing shRNA (ns) or shSmg1 (C) or shSyt17 (D) were assessed for denoted protein levels by immunoblotting. Loading is normalized to sarcomeric α-actinin. (E) NMC transduced with empty control or ectopic HIF1αΔODD in combination with ns, shSmg1 or shSyt17 were assessed for [³H]leucine incorporation. (n=3 biological replicates; shown is mean±SD; *p<0.05; one-way ANOVA followed by a Dunnet's multiple comparison post-test). (F,G) NMC expressing ectopic HIF1αΔODD in combination with ns, shSmg1 or shSyt17 were assessed at the baseline state, following an injection of oleic acid and then FCCP. Mock transduced samples serve as controls. Depicted are ECAR (F) and OCR (G) rates at the respective time-points. (n=8 biological replicates; shown is mean±SEM; *p<0.05; two-tailed unpaired t-test). (H) NMC treated as in F, G were stimulated at 1 Hz and assessed for contractile amplitude by IonOptix Contractility Recording analysis. Contractile amplitude is shown (H) (n=3 biological replicates; shown is mean±SD; *, %, p<0.05; one-way ANOVA followed by a Dunnet's multiple comparison post-test). (I) Venn-Diagram showing number of genes normalized by Sgm1 or Syt17 inactivation in hypoxic neonatal cardiomyocytes compared normoxic nsRNA controls. (J) NMC cultured and transduced as in D were assessed for denoted protein levels by immunoblotting. Loading is normalized to sarcomeric α-actinin.

FIG. 5 shows that SINT1 inactivation in vivo attenuates disease development in vivo. A) RNA derived from left ventricles of mice subjected to sham or TAC surgery was assessed for SINT1 by qPCR. All values were normalized to Hprt1 mRNA (n=3; shown is mean±SEM; *p<0.05; one-way ANOVA followed by a Dunnet's multiple comparison post-test). (B) RNA derived from left ventricles of mice subjected to sham or 1K1C surgery was assessed for SINT1 by qPCR. All values were normalized to Hprt1 mRNA (n=3 biological replicates; shown is mean±SEM; *p<0.05; one-way ANOVA followed by a Dunnet's multiple comparison post-test). (C) Schematic representation of the TAC protocol, indicating baseline echocardiography measurement performed at day=−10, sham or TAC surgery performed at day=0, scrGM and SINT1GM delivered intra venous (i.v.) at days=0-3 post-TAC and monitored by echocardiography at the indicated time-points. (D) Blood flow across non-constricted vessel or vessel constricted by TAC surgery was assessed for changes in blood velocity by Doppler imaging (n=8 mice for sham scrGM, n=7 for sham SINT1GM3, n=9 for TAC scrGM, n=12 for TAC SINT1GM3; shown is mean±SEM; *p<0.05; two-tailed unpaired t-test). (E) Left ventricle biopsies of sham and TAC mice treated with control scrGM or SINT1GM3 were assessed for HIF1a, Smg1, Upf1, phospho-Upf1 (Ser1100) and Syt17 protein level (normalized to sarcomeric α-actinin). (F) Whole hearts harvested from mice as described in C. (G,H) Longitudinal monitoring of cardiac % Ejection fraction (% EF) (G) and echocardiographically quantified left ventricular weight normalized to body weight of sham or TAC operated mice treated with scrGM or SINT1GM3 (H). Arrows in panels indicate gapmer injections as described in e (n=8 mice for sham scrGM, n=7 for sham SINT1GM3, n=9 for TAC scrGM, n=12 for TAC SINT1GM3; shown is mean±SEM; *p<0.05; two-tailed unpaired t-test). (I) Cell area quantification of laminin-stained histological sections of mice hearts after 6 weeks of sham or TAC surgery, treated with scrambled or SINT1 gapmers (n=3 mice per group; shown is mean±SD; *p<0.05; one-way ANOVA followed by Bonferroni correction). (J) Representative images of laminin-stained histological sections of mice hearts after 6 weeks of sham or TAC surgery, treated with scrambled or SINT1 gapmers. (K) Schematic representation of the 1K1C protocol, indicating baseline echocardiography measurement performed at day=−10 and 1K1C surgery performed at day=0. scrGM and SINT1GM was delivered i.v. and initiated after detection of cardiac hypertrophy at d=56. Gapmer dosage, delivery time-points and longitudinal echocardiography monitoring was performed as indicated. (L) Mean blood pressure of mice treated as described in I is shown (n=8 mice for sham scrGM, n=10 for sham SINT1GM3, n=7 for 1K1C scrGM, n=11 for 1K1C SINT1GM3; shown is mean±SEM; *p<0.05; two-tailed unpaired t-test). (M) Left ventricle biopsies of sham and 1K1C mice treated with control scrGM or SI as described in g were assessed for HIF1a, Syt17, Smg1 and Upf1 protein and phospho-Upf1 (Ser1100) (normalized to sarcomeric α-actinin). (N) Whole hearts harvested from mice as described in g, were harvested and imaged. (O,P) Longitudinal monitoring of cardiac % Ejection fraction (% EF) (O) and echocardiography quantified left ventricular weight normalized to body weight of sham or 1K1C operated mice treated with scrGM or SINT1GM3 (P). Arrows in panels indicate gapmer injections as described in g (n=8 mice for sham scrGM, n=10 for sham SINT1GM3, n=7 for 1K1C scrGM, n=11 for 1K1C SINT1GM3; shown is mean±SEM; *p<0.05; two-tailed unpaired t-test). (Q) Cell area quantification of laminin-stained histological sections of mice hearts after 13 weeks of sham or 1K1C surgery, treated with scrambled or SINT1 gapmers (n=3 mice per group; shown is mean±SD; *p<0.05; one-way ANOVA followed by Bonferroni correction). (R) Representative images of laminin-stained histological sections of mice hearts after 13 weeks of sham or 1K1C surgery, treated with scrambled or SINT1 gapmers. (S) Kaplan-Meier survival curves comparing mortality between scrGM and SINT1GM3 treated mice subjected to 1K1C. Due to absence of mortality in the sham groups, sham group data is not shown for clarity.

FIG. 6 shows that inactivation of Smg1 and Syt17 prevents hypertrophic growth and systolic dysfunction (A) Schematic representation of the AAV9-fl/fl-shRNA viruses against Smng1 and Syt17 before and after Cre-mediated recombination (left panel) and of the experimental timeline (right panel). (B, C) Left ventricular biopsies from mice subjected to sham or TAC surgery and infected with AAV9-fl/fl-shSmg1 and/or AAV9-shSyt17 were assessed for Smg1 (B) and Syt17 (C) RNA by RT-qPCR. Data was normalized to Hprt (n=3 mice per group; shown is mean±SD; *p<0.05; one-way ANOVA followed by Bonferroni correction). (D) Immunoblots of denoted proteins of mice transduced and treated as in B. (E) Left ventricular biopsies from mice subjected to sham or TAC surgery and infected with AAV9-fl/fl-shSmg1 and/or AAV9-fl/fl-shSyt17 were assessed for SINT1 by RT-qPCR. Data was normalized to Hprt (n=3 mice per group; shown is mean±SD; *p<0.05; one-way ANOVA followed by Bonferroni correction). (F) Blood flow across non-constricted vessel or vessel constricted by TAC surgery was assessed for changes in blood velocity by Doppler imaging (AAV9-fl/fl-nsRNA sham n=4, TAC=7; AAV9-fl/fl-shSmg1 sham n=3, TAC=6; AAV9-fl/fl-shSmg1 sham n=4, TAC=6; AAV9-fl/fl-shSmg1/shSyt17 sham n=4, TAC=6; shown is mean±SEM; *p<0.05; two-tailed unpaired t-test). (G, H, I) Ejection fraction (in %) (G), left ventricular posterior wall diameter in systole (LVPW; s) (H) and heart-weight/body weight ratio (HW/BW) (I) of mice treated as in F (AAV9-fl/fl-nsRNA sham n=4, TAC=7; AAV9-fl/fl-shSmg1 sham n=3, TAC=6; AAV9-fl/fl-shSmg1 sham n=4, TAC=6; AAV9-fl/fl-shSmg1/shSyt17 sham n=4, TAC=6; shown is mean±SEM; *p<0.05; two-tailed unpaired t-test). (J) Whole hearts harvested from mice as described in g, were harvested and imaged.

FIG. 7 shows that activation of the SINT1-SMG1-Syt17 axis correlates with human pressure-overload induced heart disease and its repression prevents pathologies in iPSC-derived human cardiomyocytes. (A) Human left ventricular biopsies of healthy and subjects with aortic stenosis were assessed for HIF1a, SMG1 and SYT17 protein. Loading is normalized to cardiac α-actinin. (B-D) Human left ventricular biopsies of healthy (n=3) and subjects with aortic stenosis (AS) (n=9) were assessed for SINT1 (B), SMG1 (C) and SYT17(D) RNA, normalized to HPRT1 mRNA. Results show duplicated measurements (mean±SEM; *p<0.05, ** denotes p<0.005, ***denotes p<0.0005; Mann-Whitney test). (E-G) Human left ventricular biopsies of healthy (n=3) and HCM (n=11) subjects were assessed for SINT1 (D), SMG1 (E) and SYT17(F) RNA, normalized to HPRT1 mRNA. Results show duplicated measurements (mean±SEM; *p<0.05, ** denotes p<0.005, *** denotes p<0.0005; Mann-Whitney test). (H) iPSC-hCM expressing ectopic HIF1αΔODD were treated with the respective gapmers and tested for SINT1 knockdown by qPCR. Values normalized to HPRT1 mRNA and to cardiomyocytes ectopically expressing HIF1αΔODD and treated with scrGM (n=3 biological replicates; shown is mean±SD; *p<0.05; one-way ANOVA followed by a Dunnet's multiple comparison post-test). (I) iPSC-derived human cardiomyocytes (iPSC-hCM) were transduced with an empty vector control or HIF1αΔODD and treated with scrGM or SINT1GM2 as indicated. SINT1, SMG1 and SYT17 RNA expression was examined by qPCR. (n=3 biological replicates; shown is mean±SD; *p<0.05; one-way ANOVA followed by a Dunnet's multiple comparison post-test). (J) iPSC-HCM were transduced as in I and treated with scrGM or SINT1GM4 as indicated. SINT1, SMG1 and SYT17 RNA expression was examined by qPCR. (n=3 biological replicates; shown is mean±SD; *p<0.05; one-way ANOVA followed by a Dunnet's multiple comparison post-test). (K) iPSC-hCM treated as in I were stained for cardiomyocyte-specific myosin binding protein C (MYBPC) and the membrane marker pan-cadherin, and imaged by confocal microscopy. Representative fields are shown. (L) iPSC-derived human cardiomyocytes (iPSC-hCM) transduced as in I assessed for de novo protein synthesis as readout for cardiomyocyte hypertrophy by [3H]leucine incorporation. (n=3 biological replicates; shown is mean±SD; *p<0.05; one-way ANOVA followed by a Dunnet's multiple comparison post-test). (M) iPSC-hCM transduced as in I were assessed for oxygen consumption rate (OCR) and extracellular acidification rate (ECAR) by SeaHorse Flux analysis. OCR and ECAR were assessed following an injection of glucose (Glc) followed by antimycin A (AMA) injection. The OCR to ECAR ratio of respective samples at the indicated time-points is shown. (n=8 biological replicates for mock scrGM, n=9 for mock SINT1GM2, n=10 for HIF1αΔODD scrGM and HIF1αΔODD SINT1GM2; shown is mean±SEM; *p<0.05; Dunnet's multiple comparison post-test).

FIG. 8 shows the discovery workflow. (A) Schematic representation of HIF1α-regulated eRNA discovery workflow. In parallel to genomewide epigenetic and transcriptomic data profiling for target eRNA identification, bioinformatics and in silico methods were utilized for target identification by integrating mouse ENCODE and published transcriptomic and epigenetic data focusing on Histone H3 monomethylated lysine 4 (H3K4me1), trimethylated lysine 4 (H3K4me3) and Histone H3 acetyl Lys27 (H3K27ac) chromatin immunoprecipitation-coupled sequencing (ChIP-seq) data to identify active sites of gene transcription within intergenic enhancer elements. After subtracting for transcripts overlapping with protein coding genes and transcripts corresponding to promoters of protein coding genes and annotated non-coding RNAs, including micro RNAs and long non-coding RNAs, putative eRNAs were filtered for positional conservation and sequence similarity of at least 35% between the mouse and human genome. Primers targeting a subset of the putative eRNAs were designed and left cardiac ventricular biopsies of mice were screened subjected to transaortic constriction (TAC) or 1 kidney 1 clip (1K1C) surgery, as models of human aortic stenosis- and hypertension-induced hypertrophic cardiomyopathy, respectively. eRNAs upregulated between TAC and 1K1C were further assessed for hypoxia sensitivity (B). Next, eRNAs exhibiting induction by TAC, 1K1C and hypoxia were assessed for Hif1α-sensitivity in cardiomyocytes subjected to normoxia (20% O₂) or hypoxia (3% O₂) in the presence or absence of control non-silencing shRNA (ns) or shRNA targeting Hif1α (shHif1α) (C). To complement this dataset, the inventors profiled expression of these putative eRNAs in left ventricular biopsies of control Hif1α f/f mice and of ventricular-specific Hif1α conditional knockout mice (Hif1α cKO) subjected to either sham or TAC surgery (D). eRNAs exhibiting hypoxia- and TAC-sensitivity and Hif1α-dependent expression were manually annotated for presence of conserved hypoxic response elements (HREs) upstream of their putative transcription start site (TSS). This led to the identification of 6 putative eRNAs bearing cross-species conserved HREs, which were subsequently assessed for correlation with disease in aortic stenotic and HCM human cardiac biopsies. Of the 6 identified eRNAs, e22, hereafter referred to as hypoxia-inducible factor (HIF) activated cardiac-specific eRNA (SINT1) exhibited strongest correlation specifically with the diseased patient pools. (B) qPCR profiling of a subset of eRNAs for expression in NMC cultured at 3% O2. Red line indicates level of respective eRNA in NMC cultured at 20% O2 (n=3 biological replicates; shown is mean±SD; *p<0.05; two-tailed unpaired t-test).

FIG. 9 shows epigenetic characterization and conservation of the SINT1 genomic organization. (A) NMC cultured in 20% O2 or 3% O2 and transduced with ns or shHif1α were assessed for SINT1, Smg1 and Syt17 RNA, normalized to Hprt (n=3 biological replicates; shown is mean±SD; *p<0.05; one-way ANOVA followed by a Dunnet's multiple comparison post-test). (B) Protein lysates of mouse cardiomyocytes cultured at 20% O2 or 3% O2 were probed for Hif1a, Smg1 and Syt17 protein expression by immunoblotting. Loading was normalized to sarcomeric α-actinin. (C) SINT1 expression was assessed by qPCR after co-transfection of SINT1-BoxB with the AN-SRF fusion construct in 293T cells. Data was normalized to Hprt (n=3 biological replicates; shown is mean±SD; *p<0.05; one-way ANOVA followed by a Dunnet's multiple comparison post-test compared to 293T cells expressing BoxB). (D) BoxB RNA expression was assessed by qPCR after co-transfection of SINT1-BoxB with the AN-SRF fusion construct in 293T cells. Data was normalized to Hprt (n=3 biological replicates; shown is mean±SD; *p<0.05; one-way ANOVA followed by a Dunnet's multiple comparison post-test compared to 293T cells expressing BoxB).

FIG. 10 shows that SINT1 is HIF1α-dependent enhancer template RNA. (A,B) NMC cultured at 20% O2 or 3% O2 were treated with ns or shSINT1 and stained for the cardiomyocyte specific protein sarcomeric α-actinin and DAPI and imaged by confocal microscopy (A) and, assessed for 2D cell surface area (B) (n=3 biological replicates; shown is mean±SD; *p<0.05; one-way ANOVA followed by a Dunnet's multiple comparison post-test). (C) NMC subjected to DMOG, 3% O₂ or stimulated with PE in combination with ns or shRNA targeting SINT1 was assessed for [³H]leucine incorporation. (n=4 biological replicates; shown is mean±SD; **, %, ζ, and &, p<0.05; two-tailed unpaired t-test). (D,E) NMC stimulated with DMOG, isoproterenol (Iso) or PE in combination with ns or shSINT1 were assessed for extracellular acidification rate (ECAR, D) and oxygen consumption rate (OCR, E) and by SeaHorse Flux analysis. ECAR (D) was assessed at baseline levels in the presence of glucose whereas OCR (E) was assessed followed by oleic acid injection. (n=9 biological replicates; shown is mean±SEM; *, %, ζ, and &, p<0.05; two-tailed unpaired t-test).

FIG. 11 shows SINT1 inactivation and depletion of its downstream targets inhibits transition to pathology in mouse cardiomyocytes. (A,B) NMC were subjected to 3% O2 and transduced with ns or different shRNAs targeting Smg1 or Syt17, and assessed for Smg1 (D) and Syt17 (E) expression by qPCR (n=3 biological replicates; shown is mean±SD; *p<0.05; one-way ANOVA followed by a Dunnet's multiple comparison post-test). shSmg1 #1 and #4, and shSyt17 #2 and #5 were used in downstream experiments. (C,D) NMC subjected to 3% O2 (F) or stimulated with PE (G) in combination with ns or shRNA targeting Smg1 or Syt17 was assessed for [³H]leucine incorporation. (n=4 biological replicates; shown is mean±SD; *p<0.05; one-way ANOVA followed by a Dunnet's multiple comparison post-test). (E, F) NMC cultured at 20% O2 or 3% O2 were treated with nsRNA, shSmg1 or shSyt17 and stained for the cardiomyocyte specific protein sarcomeric α-actinin and DAPI and imaged by confocal microscopy (E) and assessed for 2D cell surface area (F) (n=3 biological replicates; shown is mean±SD; *p<0.05; one-way ANOVA followed by a Dunnet's multiple comparison post-test). (G-I) OCR:ECAR ratio (G), OCR (H) and ECAR (I) quantified by Seahorse flux analysis in NMC expressing HIF1αΔODD or treated with PE in combination with ns, shSmg1 or shSyt17 (n=8 biological replicates; shown is mean±SEM; *p<0.05; two-tailed unpaired t-test). (J,K) Relative maximal velocity of contraction (J) and relaxation (K) were assessed in NMC expressing HIF1αΔODD in combination with ns, shSmg1 or shSyt17 using the IonOptix contractility analyzer (n=3 biological replicates; shown is mean±SD; *, % and ζ, p<0.05; two-tailed unpaired t-test).

FIG. 12 shows that SINT1 inactivation inhibits pathology transition in mice subjected to TAC surgery. (A) NMC expressing ectopic HIF1αΔODD were treated with the respective gapmers and tested for SINT1 knockdown by qPCR. Values normalized to Hprt1 and to cardiomyocytes ectopically expressing HIF1αΔODD and treated with control scrGM (n=3 biological replicates; shown is mean±SD; *p<0.05; two-tailed unpaired t-test). (B) Cardiomyocytes expressing ectopic HIF1αΔODD were treated with the respective gapmers and tested for Smg1 and Syt17 RNA expression by qPCR normalized to scrGM and Hprt1 (n=3 biological replicates; shown is mean±SD; *p<0.05; two-tailed unpaired t-test). (C) NMC treated with SINT1GM1 and SINT1GM3 were assessed for response to HIF1αΔODD- and PE-induced [³H]leucine incorporation. (n=3 biological replicates; shown is mean±SD; *p<0.05; one-way ANOVA followed by a Dunnet's multiple comparison post-test). (D) Validation of contraction amplitude in HIF1αΔODD transduced NMC treated with scrGM or SINT1GM3 (n=3 biological replicates; shown is mean±SD; *p<0.05; one-way ANOVA followed by a Dunnet's multiple comparison post-test). (E) RNA derived from left ventricles of mice subjected to sham or TAC surgery and treated with scrGM or SINT1GM3 was assessed for SINT1, Smg1 and Syt17 RNA, normalized to Hprt (n=3 biological replicates; shown is mean±SD; *p<0.05; one-way ANOVA followed by a Dunnet's multiple comparison post-test). (F) Physical end-point heart weight: body weight measurements of mice subjected to sham or TAC surgery (n=8 mice for sham scrGM, n=7 for sham SINT1GM3, n=9 for TAC scrGM, n=12 for TAC SINT1GM3; shown is mean±SEM; *p<0.05; two-tailed unpaired t-test compared to sham scrGM or TAC SINT1GM3). (G,H) Longitudinal analysis of left ventricular wall thickness (G) and lumen diameter of mice subjected to sham or TAC surgery (H) (n=8 mice for sham scrGM, n=7 for sham SINT1GM3, n=9 for TAC scrGM, n=12 for TAC SINT1GM3; shown is mean±SEM; p<0.05; two-tailed unpaired t-test compared to sham scrGM (*) or TAC SINT1GM3(%)). (I-L) Analysis of % Ejection fraction (I), physical end-point heart weight: body weight ratio (J) left ventricular wall thickness (K) and systolic lumen diameter (L) of mice subjected to sham or TAC surgery, and treated with SINT1GM1 (n=4 mice for sham scrGM and sham SINT1GM1, n=8 for TAC scrGM, n=10 for TAC SINT1GM1; shown is mean±SEM; *p<0.05; two-tailed unpaired t-test compared to sham scrGM (*) or TAC SINT1GM1(%).

FIG. 13 shows that SINT1 inactivation inhibits pathology transition in mice subjected to 1K1C surgery. (A) RNA derived from left ventricles of mice subjected to sham or 1K1C surgery and treated with scrGM and SINT1GM3 was assessed for SINT1, Smg1 and Syt17 RNA, normalized to Hprt (n=3 biological replicates; shown is mean±SD; *p<0.05; one-way ANOVA followed by a Dunnet's multiple comparison post-test). (B) Physical end-point heart weight: body weight measurements of mice subjected to sham or 1K1C surgery (n=8 mice for sham scrGM, n=10 for sham SINT1GM3, n=7 for 1K1C scrGM, n=11 for 1K1C SINT1GM3; shown is mean±SEM; one-way ANOVA followed by a Dunnet's multiple comparison post-test). (C,D) Analysis of left ventricular wall thickness (C) and systolic lumen diameter of mice subjected to sham or 1K1C surgery (D) (n=8 mice for sham scrGM, n=10 for sham SINT1GM3, n=7 for 1K1C scrGM, n=11 for 1K1C SINT1GM3; shown is mean±SEM; *p<0.05; two-tailed unpaired t-test compared to sham scrGM (*) or TAC SINT1GM3(%)). (E-H) Longitudinal analysis of % ejection fraction (E), physical end-point heart weight: body weight measurements (F) left ventricular wall thickness (G) and lumen diameter (h) of mice subjected to sham or 1K1C surgery, and treated with SINT1GM1 (n=3 mice for sham scrGM, n=4 for sham SINT1GM3, n=8 for 1K1C scrGM, n=9 for 1K1C SINT1GM3; shown is mean±SEM; *p<0.05; two-tailed unpaired t-test compared to sham scrGM (*) or TAC SINT1GM3(%)).

FIG. 14 shows that SINT1 activation inversely correlates with dilated cardiomyopathy (DCM) in humans and mouse models. A-C Human left ventricular biopsies of healthy (n=3) and DCM (n=6) subjects were assessed for SINT1 (A), SMG1 (B) and SYT17 (C) RNA, internally normalised to HPRT. (mean±SD; *p<0.05, ** denotes p<0.005, *** denotes p<0.0005; one-way ANOVA followed by a Dunnet's multiple comparison post-test). D, Mlp+/+ and Mlp−/− left ventricular biopsies were assessed for SINT1, Smg1 and Syt17 RNA, normalised to Hprt (n=3 left ventricles per group; shown is mean±SD; *p<0.05; one-way ANOVA followed by a Dunnet's multiple comparison post-test).

FIG. 15 shows that SINT1 activation inversely correlates with dilated cardiomyopathy in humans and mouse models. (A) stained iPSC-hCM treated as in as in FIG. 7K were assessed for 2D cell surface area (n=3 biological replicates; shown is mean±SD; *p<0.05; one-way ANOVA followed by a Dunnet's multiple comparison post-test). (B,C) iPSC-hCM treated as in FIG. 7I were assessed for ECAR (B) and OCR (C) by SeaHorse Flux analysis. OCR and ECAR were assessed following an injection of glucose (Glc) followed by antimycin A (AMA) injection and quantified. (n=9 biological replicates; shown is mean±SEM; *p<0.05; one-way ANOVA followed by a Dunnet's multiple comparison post-test). (D-F) iPSC-hCM treated FIG. 7I were assessed for contractile amplitude (D), relative maximal velocity of contraction (E) and relaxation (F) as readout for cardiomyocyte function (n=3 biological replicates; shown is mean±SD; *,% p<0.05; two-tailed unpaired t-test).

FIG. 16 shows knockdown of human SINT1 expression in iPSC-hCM expressing ectopic HIF1αΔODD. Values were normalized to HPRT1 mRNA and to cardiomyocytes ectopically expressing HIF1αΔODD and treated with scrGM (n=3 biological replicates; shown is mean±SD; all values have p<0.05 in one-way ANOVA followed by a Dunnet's multiple comparison post-test).

Table 1 qPCR primers for mouse sequences

Table 2: qPCR primers for human sequences

Table 3: Gapmer sequences directed against humanSINT1

Table 4: Gapmer sequences directed against humanSINT1. Central underscored positions are DNA; flanking sequences without underscore on either side are LNA linked by phosphothioate ester bonds.

### EXAMPLES

SINT1 Identification and Correlation with Pathology

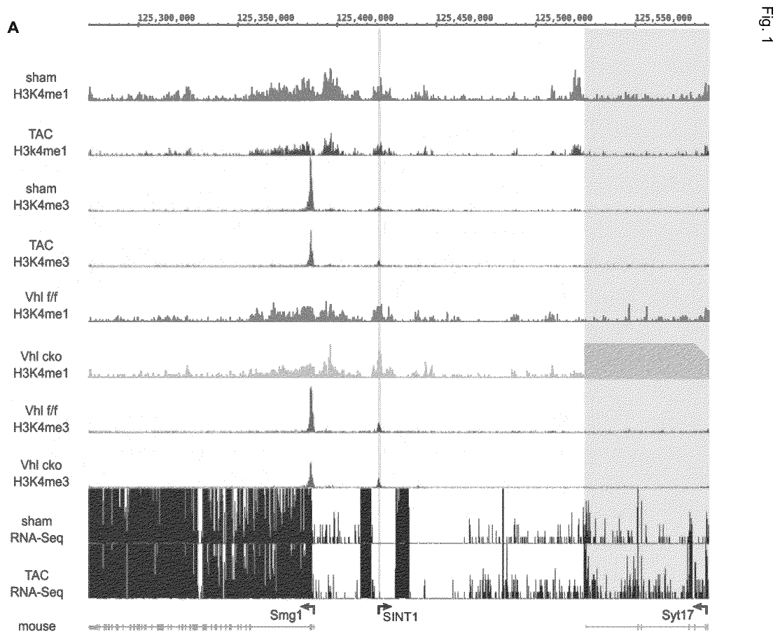

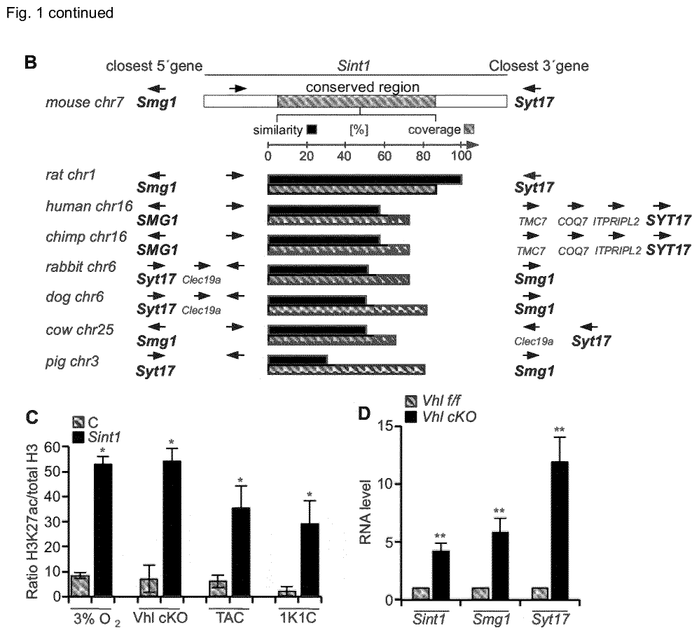

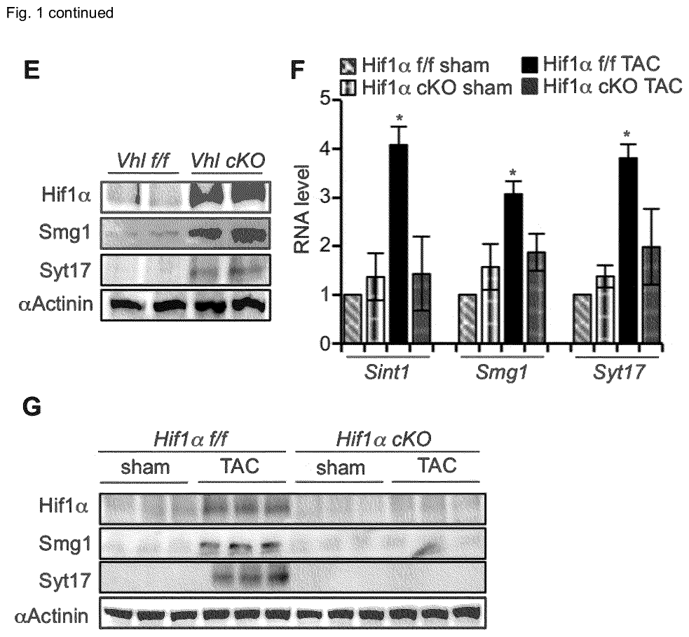

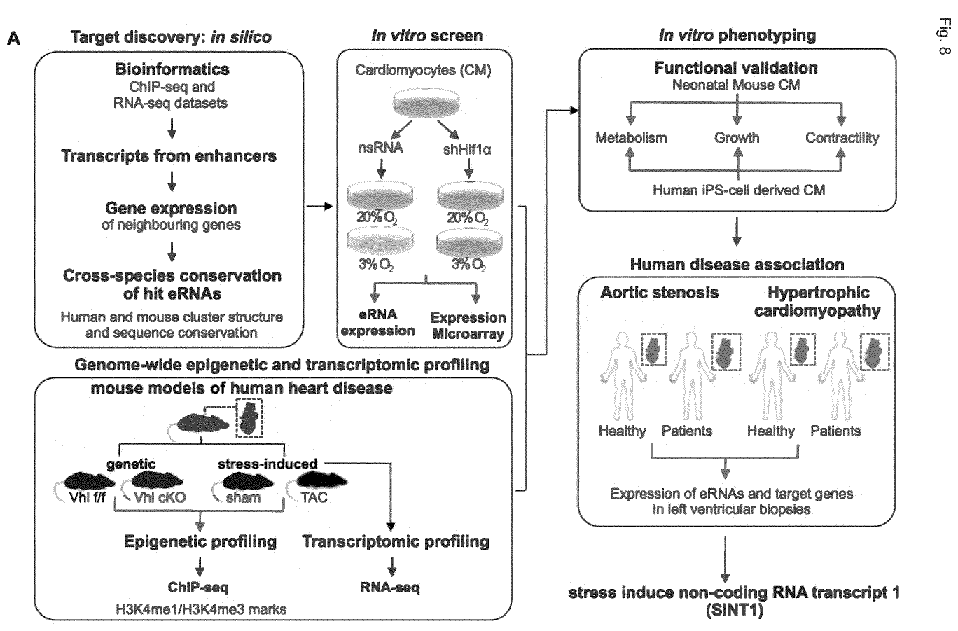

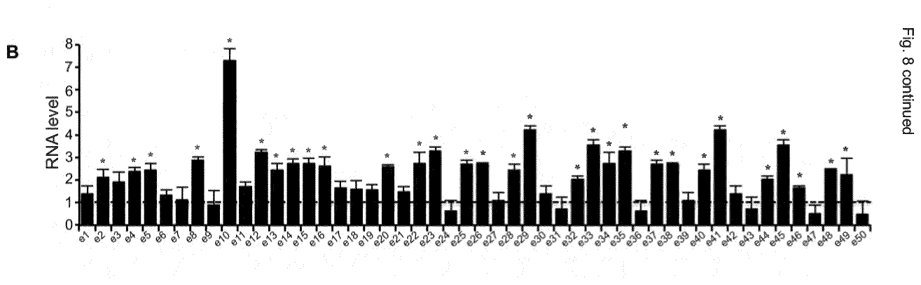

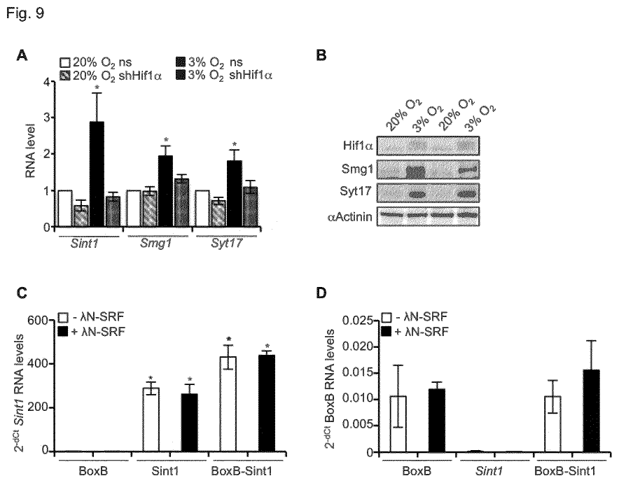

To identify potential HIF-regulated eRNAs, the inventors performed genome-wide epigenetic and transcriptomic analysis of cardiac left ventricular biopsies derived from two distinct mouse models of cardiac hypertrophy: surgery-induced aortic stenosis [transaortic constriction (TAC)] and ventricular-specific deletion of the von Hippel Lindau (Vhl) gene (referred to as Vhl cKO) (Barrick et al., Am J Physiol Heart Circ Physiol 292, H2119 (2007); Krishnan et al., Cell Metab 9, 512 (2009)). The latter genetic-based approach relies on the fact that the product of Vhl, pVhl, acts as a negative regulator of oxygen-sensitive Hifα subunits, and its deletion in the heart leads to constitutive activation of Hif1α and spontaneous cardiac hypertrophy (Krishnan et al., Cell Metab 9, 512 (2009)). Chromatin immunoprecipitation-coupled sequencing (ChIP-seq) was applied on ventricular biopsies of the above-noted models to precisely identify enhancer domains as marked by mono-methylated histone H3 lysine 4 (H3K4me1) signals, and regions of active or poised transcription as indicated by tri-methylated histone H3 lysine 4 (H3K4me3) marks. In parallel, RNA sequencing (RNA-seq) was performed on the same biopsies to detect differentially expressed transcripts. Next the inventors cross-referenced and performed differential signal analysis of the data against ENCODE and other published datasets (De Santa et al., PLoS Biol 8, e1000384 (2010); Blow et al., Nat Genet 42, 806 (2010); Mouse et al., Genome Biol 13, 418 (2012)) to select for RNAs exhibiting enhancer localization, conservation with the human genome and HIF1a dependence (described in FIG. 8 A, B). This analysis unveiled SINT1, featuring characteristics of an intergenic enhancer RNA specifically upregulated in diseased cardiac ventricles with considerable sequence conservation among mammalian species (FIGS. 1, A and B). In line with the hypoxia/HIF-sensitivity of SINT1 expression and its eRNA characteristics, ChIP-seq of left ventricular biopsies of mice subjected to TAC or 1 kidney 1-clip (1K1C) or Vhl cko mice revealed a concordant increase in H3K4me3 signal at the SINT1 locus (FIG. 10), a finding that is also consistent with elevated H3K4ac27 marks observed by ChIP in neonatal mouse cardiomyocytes (NMC) subjected to hypoxia and in mouse disease models including TAC, Vhl cKO and the 1 kidney 1-clip (1K1C) model (FIGS. 1, A and C). Intriguingly, these analyses also revealed parallel changes in mRNA and protein expression of the SINT1-flanking genes Smg1 and Syt17 and conservation of this gene cluster structure in various species (FIG. 1, B, D to G). SINT1 induction occurred concomitant to elevated Smg1 and Syt17 expression in independent mouse cohorts subjected to mouse surgical and genetic models of cardiomyopathy, and in primary rodent cardiomyocytes exposed to hypoxia and transduced with scrambled short-hairpin RNA (shRNA) or shRNA targeting Hif1α (FIG. 1, D to G and FIGS. 9, A and B).

SINT1 is a HIF1α-Dependent Non-Coding RNA with Enhancer Function

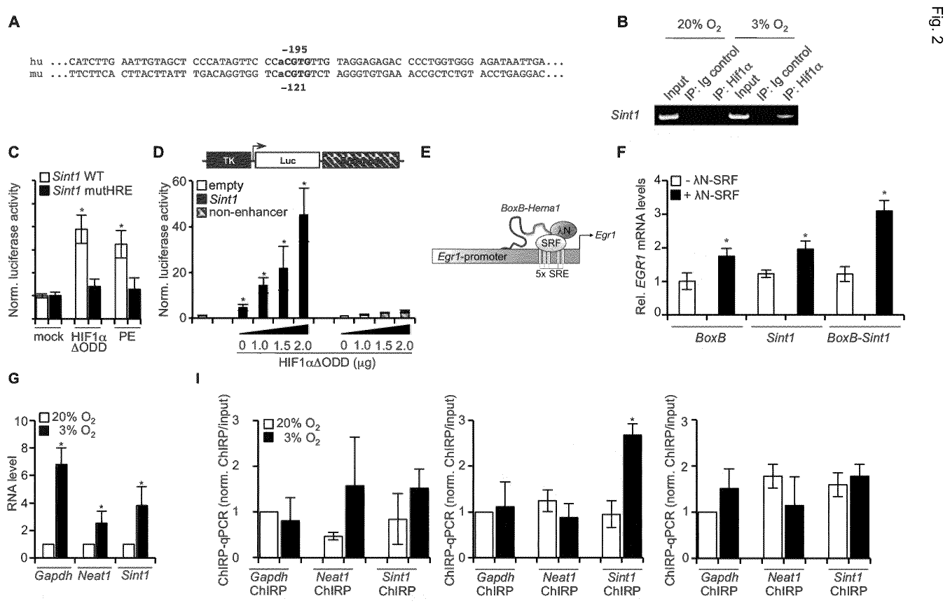

As SINT1 transcription correlated with Hif1α activation, the inventors investigated if SINT1 is a direct target of Hif1a. In silico analysis of the SINT1 promoter revealed a conserved hypoxia response element (HRE) at position −121 bp and −195 bp upstream of the transcription start site (TSS) in the mouse and human genome, respectively (FIG. 2A). To assess the functionality of this HRE, the inventors performed Hif1α ChIP from nuclear extracts of NMC subjected to normoxia (20% O₂) or hypoxia (3% O₂). Hif1α was associated at the HRE of the SINT1 promoter in native chromatin only in NMC subjected to hypoxia (FIG. 2B). Moreover, mutation of the conserved SINT1 HRE resulted in blunted SINT1 promoter-luciferase reporter activity in response to ectopic expression of HIF1α lacking the oxygen-dependent degradation domain (HIF1αΔODD) or to stimulation with the hypertrophy-inducing α-adrenergic receptor agonist phenylepinephrine (PE) (FIG. 2C). Conserved HREs were not detected in promoters of Smg1 or Syt17, and Hif1α ChIP with primers targeting 150-200 bp segments of either promoter failed to yield a discernible signal and luciferase reporter assays with the Smg1 or Syt17 promoter also failed to demonstrate hypoxia or Hif1α sensitivity (data not shown). To assess enhancer function of SINT1, the inventors cloned a 2.9 kb fragment containing SINT1 and additional flanking sequences including the HRE, downstream of the SV-40 promoter driving luciferase expression (Lam et al., Nature 498, 511 (2013)), and quantified luciferase activity in response to HIF1αΔODD expression (FIG. 2D). Reporter assays revealed a Hif1α dose-dependent increase in enhancer activity of the luciferase promoter reporter while negligible effects on luciferase activity was observed with a 3 kb control genomic fragment encompassing the peroxisome proliferator activated receptor γ (Pparγ) promoter void of enhancer features. Finally, in order to confirm the innate capacity of SINT1 to increase target gene expression, the inventors utilized the λN-BoxB tethering-based reporter assay (Baron-Benhamou et al., Methods Mol Biol 257, 135 (2004)). A chimeric RNA containing SINT1 fused to BoxB RNA was engineered to facilitate recruitment of the BoxB-SINT1 RNA fusion to the RNA binding domain of λN protein fused to the serum response factor (Srf) gene (λN-Srf) (FIG. 2E). Thus, SINT1 can be artificially tethered to SRF transcription factor response elements (SREs) within the early growth response gene 1 (Egr1) promoter (FIG. 2E) (Pagel et al., Indian J Biochem Biophys 48, 226 (2011). The inventors observed that the BoxB-SINT1 RNA fusion significantly increased Egr1 mRNA expression compared to transfection of either BoxB or SINT1 alone (FIG. 2F and FIGS. 9, C and D). These data further support the fact that the SINT1 transcript itself directs flanking gene transcription upon Hif1α-induced activation and is consistent with the functional mechanism of a documented estrogen receptor α (ERα) eRNA (Li et al., Nature 498, 516 (2013)).

Given that SINT1 expression parallels that of Smg1 and Syt17, the inventors asked if the SINT1 transcript interacts with the promoters of Smg1 and Syt17 to drive transcription. To that end, the inventors quantified SINT1 binding at the Smg1 and Syt17 promoter by Chromatin Isolation by RNA purification (ChIRP) (Chu et al., Mol Cell 44, 667 (2011)), utilizing glyceraldehyde-3-phosphate dehydrogenase (Gapdh) and nuclear paraspeckle assembly transcript 1 (Neat1), established coding and non-coding hypoxia targets (Graven et al., J Biol Chem 269, 24446 (1994); Choudhry et al., Oncogene, (2014), respectively, as promoter controls for the efficiency and specificity of SINT1 interaction. After cross-linking endogenous RNA to its target, the inventors precipitated SINT1 with biotinylated oligonucleotides and performed qPCR on SINT1 ChIRP pull-down products (FIGS. 2, G and H). Despite pronounced hypoxia-induced Gapdh and Neat1 expression (FIG. 2G), SINT1 binding was not detected at the Gapdh or Neat1 promoters but mainly at the Smg1 and Syt17 promoters (FIG. 2H), which is suggestive of selective SINT1 interaction at these promoters. To better define sites of SINT1 interaction at the respective promoters, the inventors sub-divided the Smg1 and Syt17 promoters into three 500 kb domains, designed probes and assessed interactions of the respective RNAs by ChIRP-qPCR (FIG. 2I). Gapdh and Neat1 ChIRP pull-down products served as specificity controls for SINT1 interactions. A clear signal was detected for SINT1 at the Smg1 and Syt17 promoters between −1000 to −500 bp and −500 to 0 bp upstream of the respective TSS (FIG. 2I). ChIRP pull-down of Gapdh and Neat1 RNA did not reveal such robust interactions at the Smg1 (FIG. 2I upper panels) or Syt17 promoters (FIG. 2I lower panels). Slight binding was observed for Neat1 at the 5′ domain of Smg1 but was statistically insignificant (FIG. 2I, upper panels). Fluorescence in situ hybridization (FISH) for SINT1 and its flanking genes revealed co-regulation and co-localization of all transcripts from the Smg1-SINT1-Syt17 gene cluster in isolated cardiomyoctes cultured at 3% O2 (FIG. 2J). Together, these results suggest that SINT1 acts at nearby genes to promote their transcriptional activation in a hypoxia-dependent manner.

SINT1 is Necessary for Pathology-Induced Smg1 and Syt17 Activity

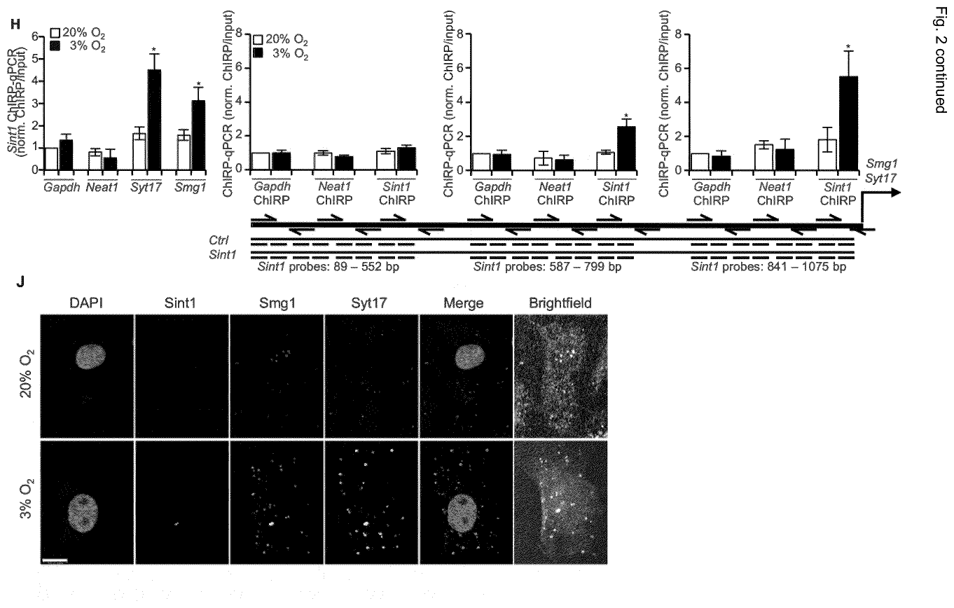

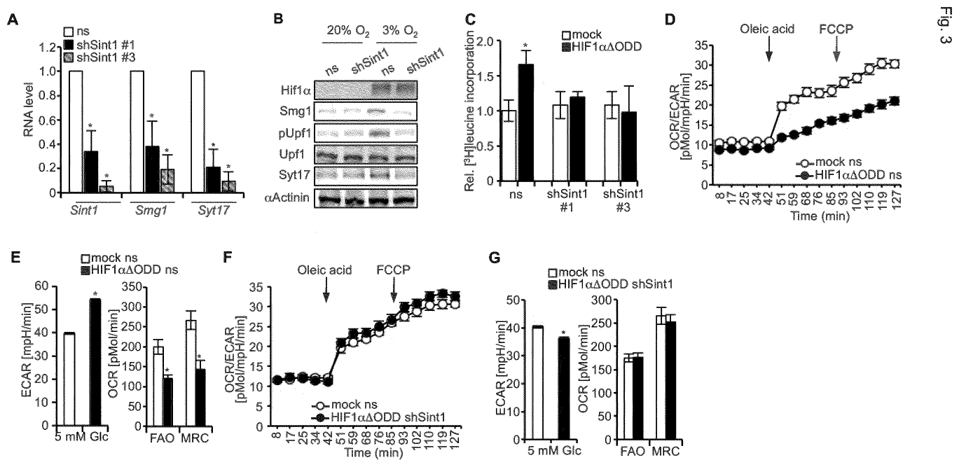

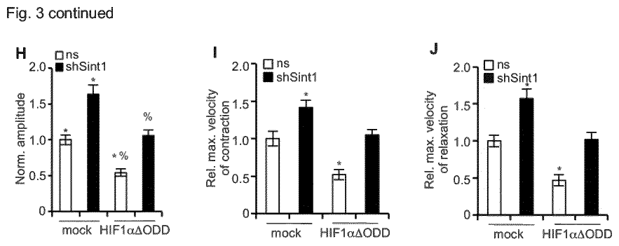

To systematically ascertain the function of the SINT1, the inventors generated short-hairpin RNAs (shRNA) targeting mouse SINT1 (shSINT1), of which two individual clones efficiently inhibited hypoxia-induced SINT1 expression resulting in the concordant upregulation of Smg1 and Syt17 in NMC on the RNA and protein level (FIGS. 3, A and B). In line with the repression of Smg1, phosphorylation of the Smg1 substrate Upf1 was abolished (FIG. 3B). Next, the inventors assayed for SINT1-mediated cell growth by [³H]leucine-incorporation and 2D cell size quantification in NMC subjected to either hypoxia, dimethyloxaloylglycine (DMOG; a chemical inhibitor of prolyl hydroxylases), PE stimulation or ectopic HIF1αΔODD expression. SINT1 depletion suppressed in each case pathologic stress-induced cell growth (FIG. 3C and FIG. 10, A to C), suggesting that this eRNA is a critical downstream effector of Hif1α important for enacting a pathologic growth response. The shift to the disease state in heart cells is characterized not only by the deregulated cell growth, but also by increased glucose utilization and dependence (through glycolysis), reduced oxidative phosphorylation capacity and depressed cardiomyocyte contractility. To interrogate SINT1 function in these contexts, NMCs expressing ectopic HIF1αΔODD, were transduced with shSINT1 and kinetic analysis of glycolytic and fatty acid oxidation (FAO) rates was determined by SeaHorse Flux analysis. As noted in FIGS. 3, D and E, ectopic HIF1αΔODD expression led to increased glycolysis, and depression of FAO and maximal respiratory capacity (MRC), as readout for mitochondrial respiratory function. However, upon simultaneous SINT1 inactivation, the shift to glycolysis at the expenses of FAO and mitochondrial function was severely attenuated (FIGS. 3, F and G). A similar response was observed in DMOG, isoproterenol or PE treated NMCs upon shSINT1 infection (FIGS. 10, D and E). shSINT1 treatment also reverted the contractile defects associated with pathologic transition, revealing a beneficial positive inotropic and lusitropic effect of SINT1 inhibition both at the basal state and upon HIF1αΔODD expression (FIG. 3, H to J). These data reveal a critical requirement of SINT1 function in mediating the maladaptive growth, metabolic and contractile changes associated with pathology.

Smg1 and Syt17 Drive Distinct Aspects of Pathologic Transition

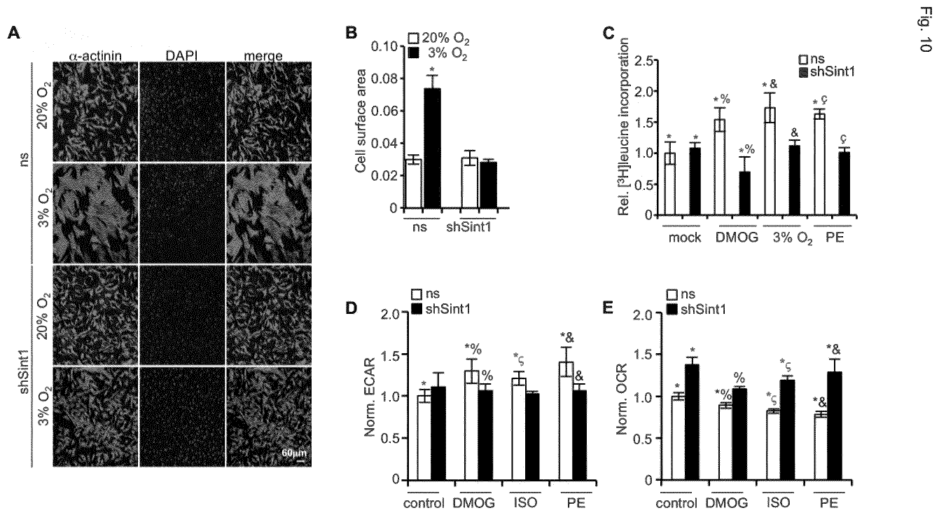

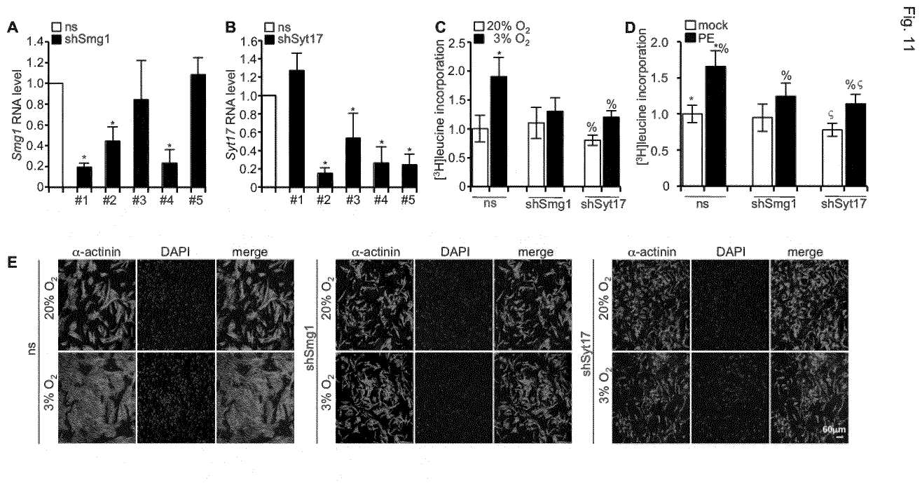

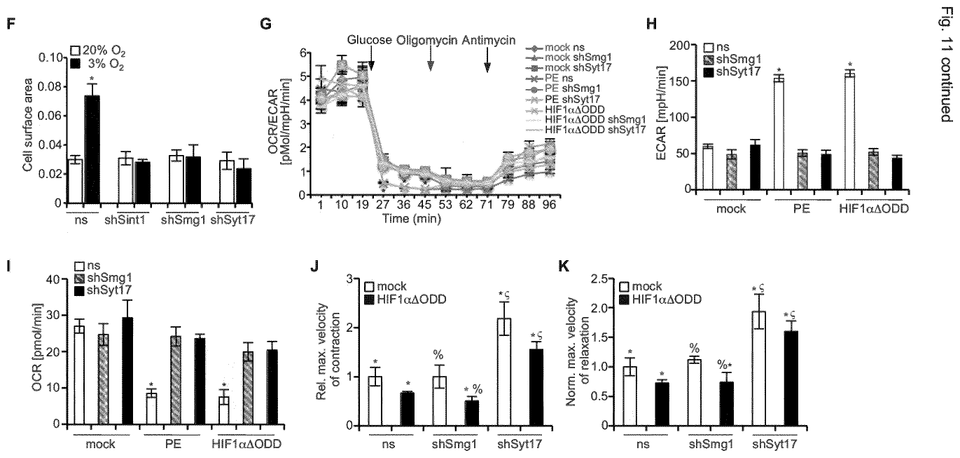

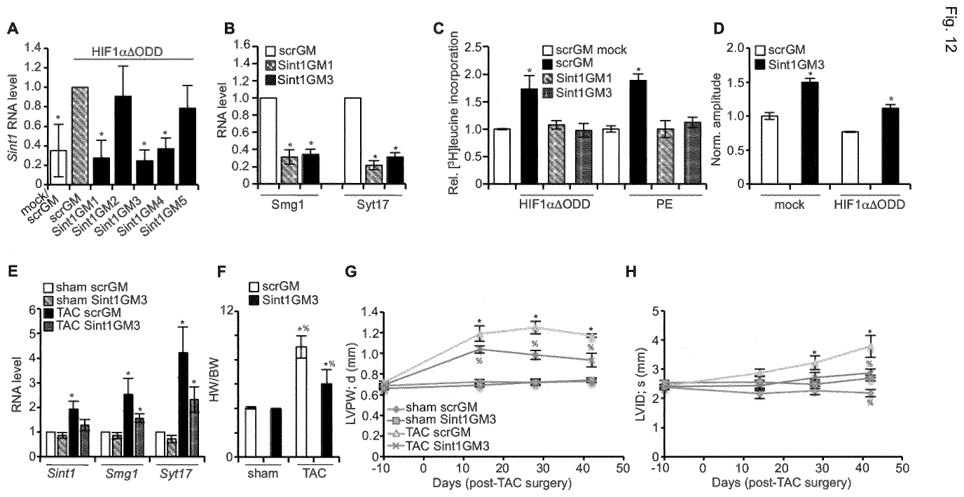

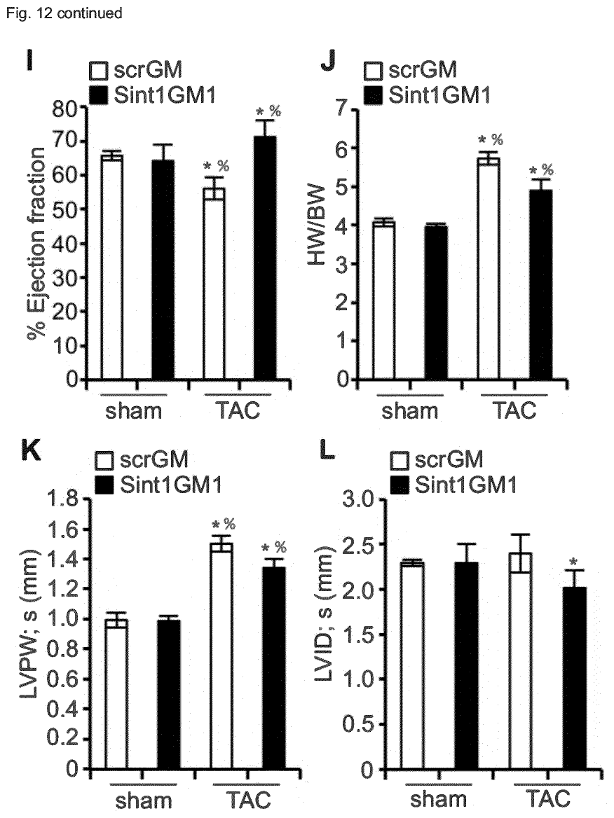

As little is known as to Smg1 and Syt17 function in heart cells, the inventors characterized the sub-cellular localization of the respective proteins in NMCs. As shown in FIG. 4A, Smg1 is primarily localized in the nuclei of cardiomyocytes while Syt17 co-localizes with Atp5a1 (a core component of ATP Synthase/Complex V), indicative of mitochondrial localization (FIG. 4B). Based on this finding and the fact that Smg1 and Syt17 represent targets of SINT1 eRNA, shRNAs targeting Smg1 and Syt17 were identified which effectively inhibited Smg1 and Syt17 expression, respectively, at the RNA and protein level (FIGS. 4, C and D and FIGS. 11, A and B). shRNAs targeting Smg1 and Syt17 were transduced into cells subjected to hypoxia, ectopic HIF1αΔODD expression or PE stimulation. In each setting, depletion of Smg1 and Syt17 prevented a hypertrophic cell growth response (FIG. 4E, and FIG. 11, C to F), indicating that Hif1a, SINT1 and Smg1-Syt17 act in a pathway critical for cardiomyocyte growth in response to pathologic stressors. Moreover, measurements of metabolic parameters, expressed as oxygen consumption (OCR): extracellular acidification (ECAR) ratios, in NMC that were depleted for Smg1 or Syt17 in combination with either HIF1αΔODD overexpression or treatment with DMOG, isoproterenol or PE revealed a requirement of Smg1 or Syt17 for efficient reprogramming of cardiomyocyte metabolism towards glycolysis in the context of stressors activating HIF signaling (FIGS. 4, F and G, and FIG. 11, G to I). In contrast to the contribution of both Smg1 and Syt17 to pathologic growth and metabolism, Smg1 inactivation did not affect HIF1αΔODD-mediated contractile repression, while treatment with shRNA targeting Syt17 effectively rescued the decrease in contractile amplitude, maximal velocity of contraction and relaxation affected by HIF1αΔODD (FIG. 4, I, and FIGS. 11, J and K). In attempting to identify downstream molecular changes consequent of Smg1 or Syt17 inhibition, the inventors performed RNAseq and analyzed differential gene expression in cardiomyocytes cultured in normoxia or hypoxia, concomitant to Smg1 or Syt17 inactivation. Although this analysis provided an extensive list of differentially expressed genes, the inventors sought to simplify the data by specifically isolating gene subsets that were: induced in hypoxia (relative to normoxia nsRNA controls) but normalized by either Smg1 or Syt17 inactivation; or repressed in hypoxia (relative to normoxia nsRNA controls) but normalized by either Smg1 or Syt17 inactivation. In categorizing differentially expressed genes in this manner the inventors isolated and identified specific gene subsets that directly inhibit the hypoxia-driven growth, metabolic and contractile maladaptation. As noted in FIG. 41, gene expression of a larger number of genes were normalized to normoxia control levels by Smg1 inactivation (compared to Syt17). Functional clustering of genes normalized by both of Sgm1 and Syt17 inactivation revealed an enrichment for genes implicated in growth and metabolic control, while individually, Smg1 and Syt17 inactivation also led to the normalization of other metabolic and growth control genes FIGS. 12, A and B). Furthermore, consistent with the in vitro contractility analysis, Syt17 knockdown had a specific impact on normalization of RNA levels of genes linked to cardiomyocyte contractility. In consequence, the inventors investigated expression of Serca2 and Phospholamban as well as Phospholamban phosphorylation at Ser16 in lysates of NMC cultured under normoxic or hypoxic conditions transduced with shNs or shSyt17. Hypoxia led to a clear downregulation of Serca2 expression as well as reduced phosphorylation of Phospholamban in shNs transduced NMC, whereas Serca2 protein expression was increased upon shSyt17 depletion in normoxia and hypoxia (FIG. 4J). Thus, SINT1-dependent Smg1 and Syt17 transcriptional activation in response to pathologic stressors, engages at a minimum two pathways with distinct and independent roles in cardiomyocyte growth, metabolism and contractility regulation.

SINT1 Inactivation In Vivo Attenuates Disease Development In Vivo

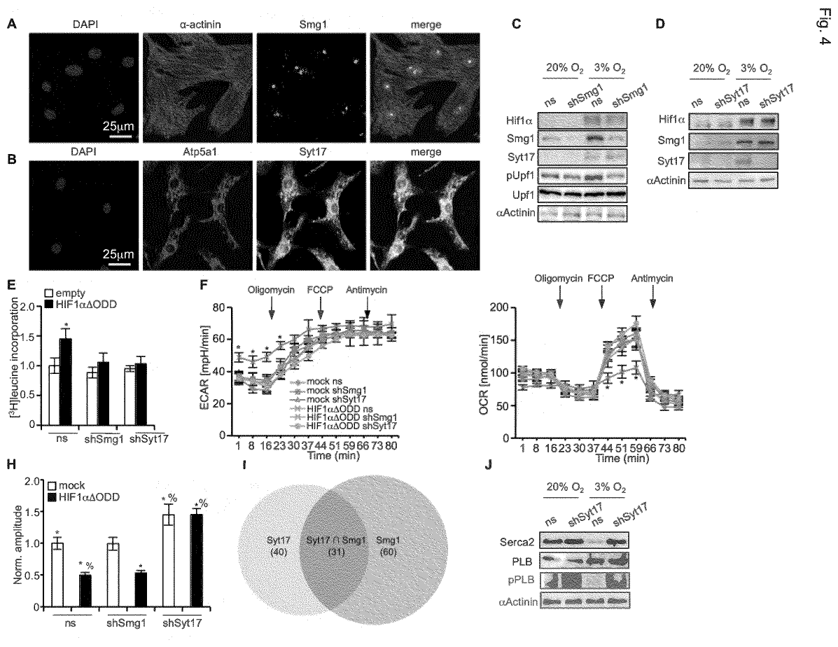

Enhancers are established modulators of spatio-temporal gene expression and eRNA templated at these regions can potentially exhibit tissue- and context-specific gene expression. Hence, the inventors assessed tissue distribution of SINT1 expression in mice subjected to aortic stenosis-induced hypertrophy (TAC) or hypertension-induced hypertrophy (1K1C). As noted in the qPCR analysis (FIGS. 5, A and B), SINT1 expression was dramatically elevated in the left ventricle of mice only upon stress induction, while expression in other tissue, including the cardiac atria, remained low both in control sham mice and in mice subjected to TAC or 1K1C. To define SINT1 function in vivo in the context of heart disease, the inventors first screened for ASO gapmers that would efficiently target SINT1 for degradation in NMC ectopically expressing HIF1αΔODD (FIG. 12A). In accord with the above-noted findings, SINT1 gapmers inhibited hypoxia-induced Smg1 and Syt17 expression in NMC, suppressed hypertrophy in cells expressing ectopic HIF1αΔODD or stimulated with PE, and rescued Hif1α-mediated contractile inhibition (FIG. 12 B to D), confirming the efficacy of the identified gapmers. These gapmers were then applied in two models of cardiomyopathy to investigate potential contributions of SINT1 to heart disease development and maintenance. With respect to the former, scrambled and SINT1 targeting gapmers were delivered prior to TAC surgery and for the next 3 consecutive days as depicted in FIG. 5C. The mice were assessed for 42 days post-TAC with echocardiography performed at regular 14-day intervals. TAC surgery led to a comparable increase in aortic flow velocity in the respective TAC groups, while sham treated mice displayed basal aortic velocity (FIG. 5D). Consistent with effects in vitro, SINT1GM3 delivery in mice resulted in blunted SINT1 levels, reduced Smg1 and Syt17 expression and attenuated phosphorylation of Upf1 (FIG. 5E and FIG. 12E). Sham operated mice treated with scrGM or HernaGM3 did not display significant changes in cardiac function, while mice subjected to TAC and treated with scrGM exhibited pronounced decline in cardiac function, increased hypertrophy and ventricular dilatation from 14-days post-TAC (FIG. 5, F to J, FIG. 12 F to H). In contrast, TAC mice treated with SINT1GM3 exhibited a blunted response to the induction of ventricular dilatation and hypertrophy, whilst maintaining cardiac function up to 42-days post-TAC surgery (FIGS. 5, G and H, FIGS. 12 G and H). These findings were recapitulated using an independent SINT1 targeting ASO gapmer, SINT1GM1 (FIG. 12 I to L). Hence, SINT1 function is critical for the development of pathologic-stress induced hypertrophic heart disease.

Next, the inventors interrogated SINT1 function in mice exhibiting overt indications of pathologic growth, dilatation and contractile dysfunction in order to evaluate the therapeutic implications of SINT1 inhibition. C57Bl/6 mice were randomly assigned into two groups, with the groups subjected to either sham or 1K1C surgery and further subdivided for scrGM or SINT1GM3 treatment upon pathology development (FIG. 5K). The 1K1C protocol leads to cardiomyopathy subsequent to the development of hypertension via the sympathetic system, angiotensin-converting enzyme activity and Na²⁺/H₂O retention (Lu et al., J Am Soc Nephrol 21, 993 (2010)). 1K1C surgery was performed and hypertrophy allowed to progress until overt indications of cardiac dysfunction was observed by echocardiography, at which time gapmer treatment was initiated and echocardiography performed at regular intervals to monitor disease progression (FIG. 5K). Blood pressure was assessed to confirm elevation in blood pressure upon 1K1C application (FIG. 5L). SINT1GM3 led to inhibition of pathology-induced SINT1, Syt17, Smg1 expression and reduced Upf1 phosphorylation (FIG. 5M and FIG. 13A). At 56-days post-1K1C, cardiac dysfunction was observed in the 1K1C group and mice from the respective groups further subdivided for scrGM or SINT1GM3 therapy. Following disease progression by echocardiography, sham operated mice did not display evidence of pathology, while 1K1C operated mice treated with scrGM displayed a progressive decline in cardiac function as evidenced by hypertrophy development, ventricular dilatation and reduced cardiac ejection fraction throughout the 91 day duration (FIG. 5, N to S, FIG. 13 B to D). However, 1K1C mice treated with SINT1GM3 demonstrated resolution of disease-associated pathologies including gradual improvement of cardiac function and reversion of hypertrophic cardiac growth (FIG. 5, O to R and FIGS. 13 C and D). Notably, despite the protection conferred by SINT1GM3 in 1K1C mice, a mild increase in physical heart weight was detected at the end of the protocol (FIG. 5P and FIG. 13C), though this was not reflected in 2D surface area measurements of cardiomyocytes from these hearts (FIGS. 5, Q and R). In accord with the echocardiography measurements, mice from the 1K1C scrGM treated group displayed reduced overall survival compared to 1K1C mice treated with SINT1GM3 (FIG. 5S). These finding were confirmed using an independent SINT1 targeting ASO gapmer, SINT1GM1 (FIG. 13 E to H). Thus, pathologic-stress induced SINT1 expression is critical for maintaining key aspects of hypertrophic heart disease-associated pathologies in mouse models. In this regard, analysis of the murine ENCODE dataset revealed the restricted expression of SINT1 to the heart.

SINT1 Function can be Uncoupled Via Smg1 and Syt17 In Vivo

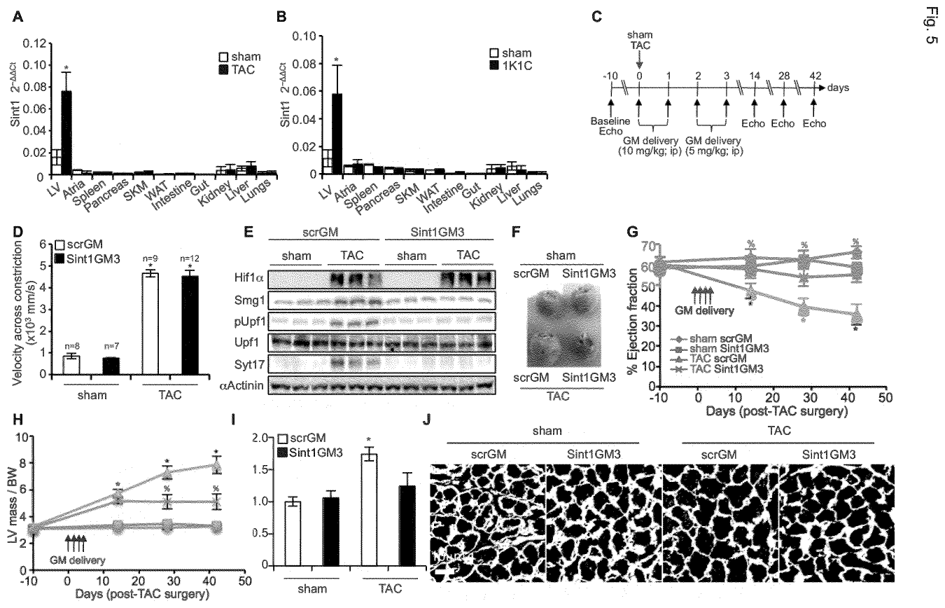

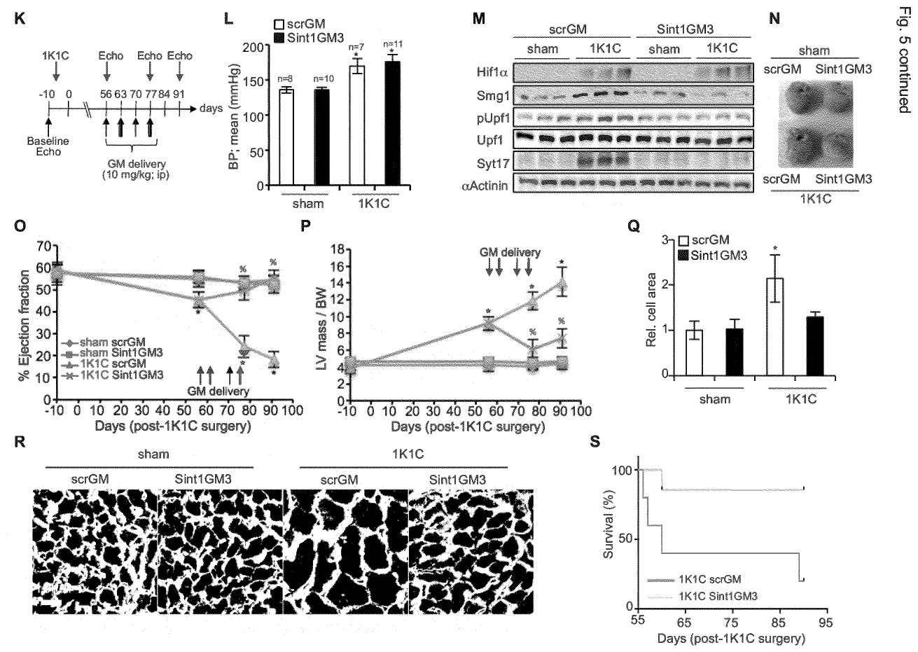

In vitro phenotypic analysis and gene expression profiling of Smg1 and Syt17 function revealed a cooperative role of both genes in normalizing dysregulated cardiomyocyte growth and metabolism but a unique capacity of Syt17 in correcting the maladaptive contractility induced by pathologic stress (FIG. 4 H). To assess if these SINT1-flanking genes confer similar effects in vivo, the inventors subjected mice expressing Cre recombinase under the control of the ventricle specific myosin light chain 2v (MLC2v) promoter (MLC2v-cre/+, (Chen et al., Development 125, 1943 (1998))) to TAC and delivered a modified adeno-associated virus 9 (AAV9) where shRNA transcription is dependent on Cre recombinase activity, thus restricting shRNA expression to the cardiac ventricle. AAV-shRNAs targeting either Smg1 (AAV9-fl/fl-shSmg1) or Syt17 (AAV9-fl/fl-shSyt17) were administered individually or simultaneously as depicted in FIG. 6A (Mirtschink et al., Nature 522, 444 (2015)). A scrambled non-silencing RNA construct was used as control. As shown in FIG. 6, B to D, efficient mRNA and protein knockdown of the respective targets was achieved by AAV9-mediated shRNA cardiac transduction, while SINT1 RNA levels were maintained (FIG. 6E). Thereafter, mice treated with the respective shRNAs were subjected to sham or TAC surgery. As indicated in FIG. 6F, TAC surgery significantly increased blood blow across the aorta of mice of all groups to a similar extent. At 4-weeks post-surgery, mice were assessed by echocardiography and a dramatic decline in cardiac systolic function was observed in control mice injected with an AAV9 bearing a non-silencing shRNA (AAV9-fl-fl-nsRNA) and in mice inactivated for Smg1 in the myocardium (FIG. 6G). In contrast, mice treated with AAV9-fl/fl-shSyt17, to inactivate Syt17 in the myocardium, revealed normal cardiac function despite TAC-induced pressure-overload (FIG. 6G). In line with these results lysates of left ventricular biopsies from TAC-operated and AAV9-fl/fl-shSyt17 treated mice, show normalized Serca2 protein levels as well as increased Ser16-phosphorylation of Phospholamban compared to sham or TAC-operated mice injected with AAV9-fl-fl-nsRNA (FIG. 6D). Left ventricular posterior wall thickness as an indicator of pressure-overload induced hypertrophy was largely normalized by simultaneous inactivation of Smg1 and Syt17 (FIG. 6H). In line with these results physical dimensions of the heart were partially normalized in TAC-operated mice inactivated for Smg1 and Syt17 in the myocardium when compared to TAC-operated mice treated with AAV9-nsRNA (FIGS. 6, I and J). Thus, simultaneous inactivation of the flanking genes led to normalization of all aspects of cardiac function, dimension and morphology, hence recapitulating SINT1 in vivo inactivation.

SINT1 Correlates with Human Cardiac Hypertrophy and is Necessary for Disease Transition

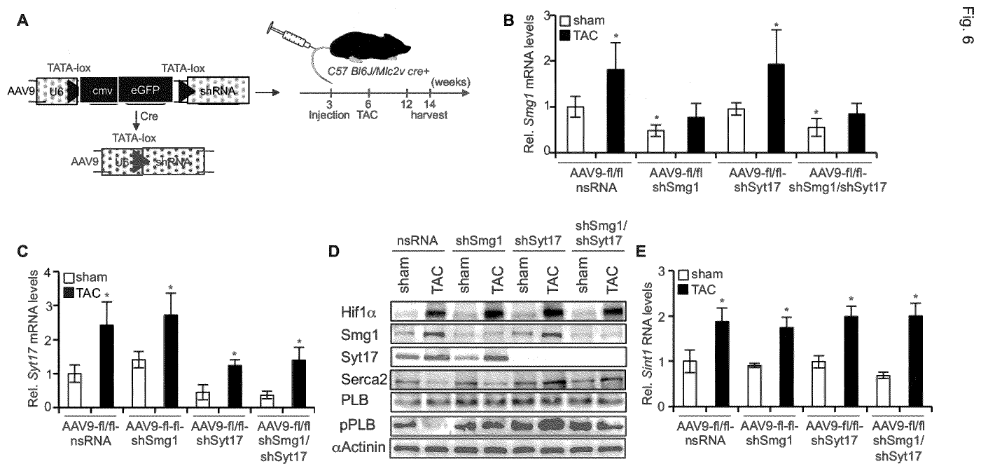

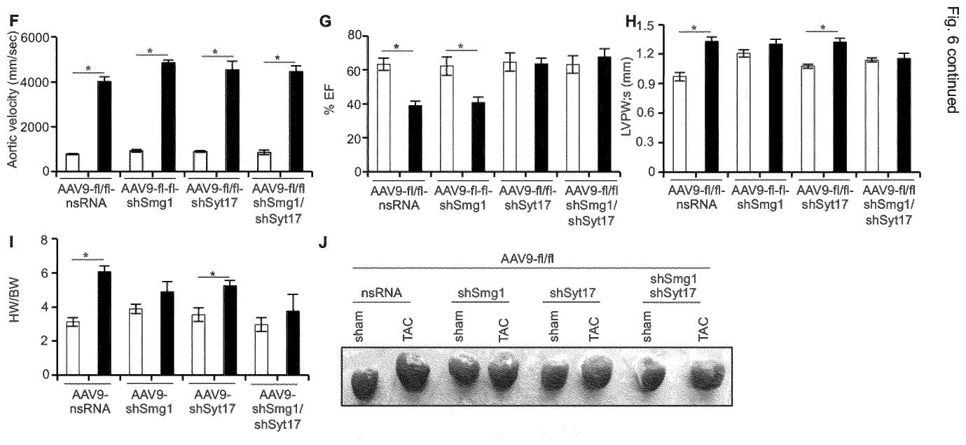

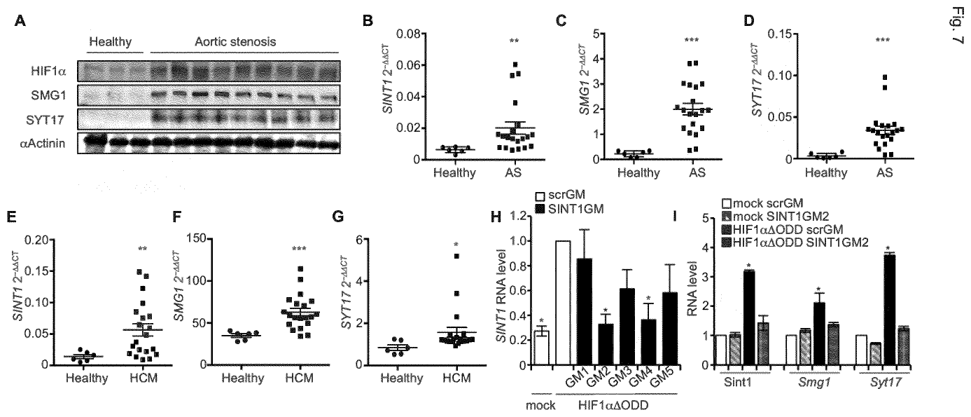

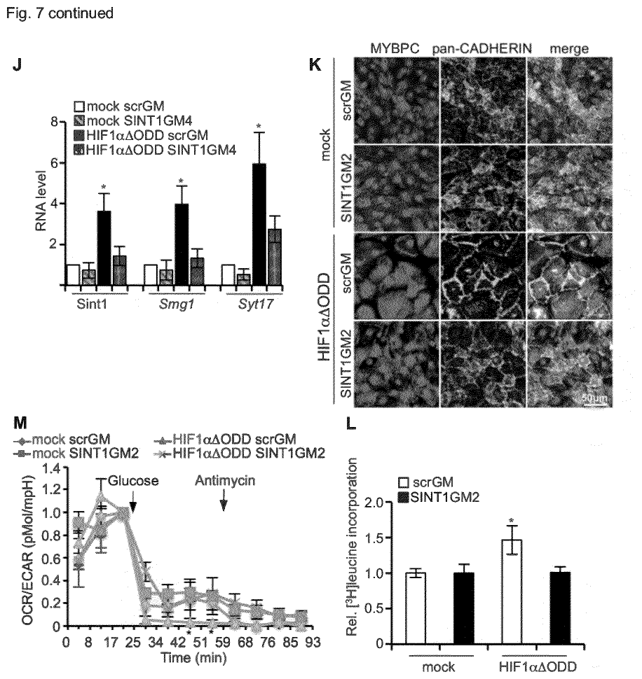

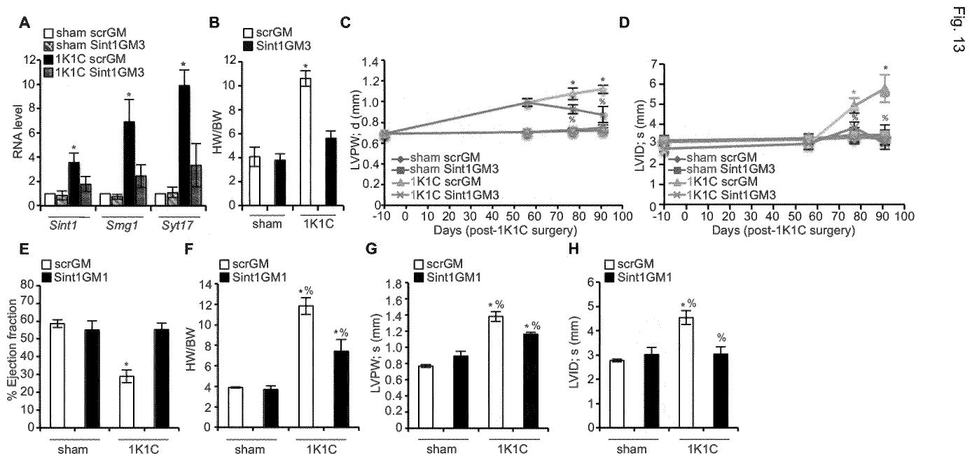

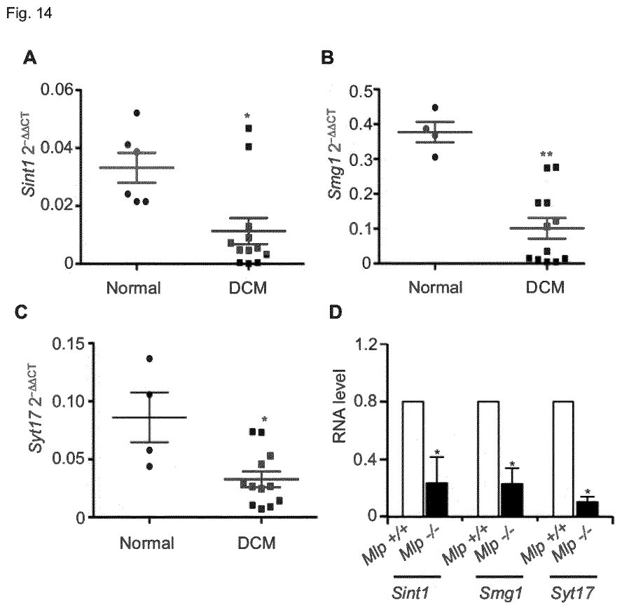

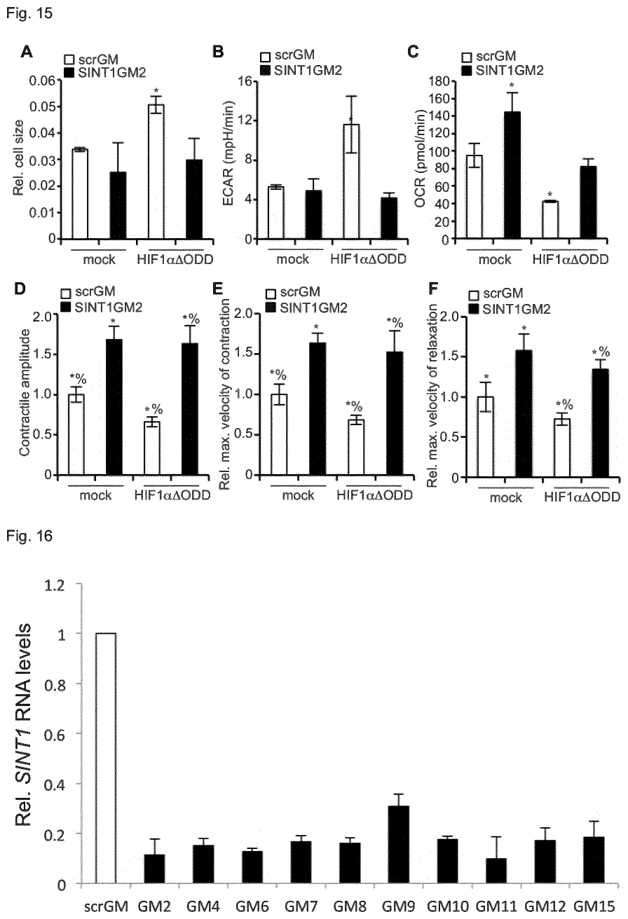

The in-silico analysis indicated conservation of this gene cluster structure in various species, (FIG. 1B), including humans, where SINT1 is similarly flanked by SMG1 and SYT17 in the genome and shares 38% overall sequence similarity with the mouse homolog (FIG. 1B). Hence, the inventors interrogated SINT1 function in human cardiomyopathic samples. SINT1 induction occurred concomitant to elevated SMG1 and SYT17 expression in independent patient cohorts of idiopathic hypertrophic cardiomyopathy (HCM) and aortic stenosis-induced cardiomyopathy (Mirtschink et al., Nature 522, 444 (2015)) (FIG. 7, A to G). In contrast, the inventors detected an inverse correlation of the SINT1-SMG1-SYT17 axis in ventricular biopsies of patients with dilative cardiomyopathy (DCM) and in ventricular biopsies of a Muscle LIM protein (Mlp)−/− DCM mouse model (Arber et al., Cell 88, 393 (1997)) (FIG. 14 A-D) likely pointing to an etiology-specific basis for SINT1 function. To determine potential causality between SINT1 transcription, and Smg1 and Syt17 expression, the inventors identified RNase H-activating stabilized anti-sense oligonulceotides (ASOs) ‘gapmer’ targeting SINT1 for knockdown in iPSC-derived human cardiomyocytes (iPSC-hCM). Screening for ASO gapmers targeting SINT1 in iPSC-hCM ectopically expressing HIF1αΔODD (Huang et al., Proc Natl Acad Sci USA 95, 7987 (1998).) identified gapmers 2 and 4 as effective tools to suppress SINT1 production (FIG. 7H). Delivery of SINT1-targeting GM2 (SINT1GM2) in iPSC-hCM suppressed SINT1 expression and attenuated SMG1 and SYT17 induction, with similar results observed using SINT1GM4 (FIGS. 7, I and J). To assess its impact on pathologic hypertrophy, the inventors depleted SINT1 in iPSC-hCM expressing ectopic HIF1αΔODD and quantified leucine-incorporation and 2D cell size as readouts for cell growth. Notably, SINT1GM2 inhibited cardiomyocyte hypertrophy (FIGS. 7K and L, and FIG. 16A). Next, the inventors assayed SINT1 function in HIF-driven metabolic reprogramming and cardiomyocyte contractility. As shown in FIGS. 7, M and N, and FIG. 16 B to F, SINT1GM2 suppressed the pathologic shift to glycolysis and the negative effects on cardiomyocyte contractility and relaxation that are normally caused by HIF1αΔODD expression. Taken together, the data obtained from left ventricular biopsies of patients suffering on pressure-overload induced heart failure recapitulate the expression profile of the translational mouse models of left ventricular pressure overload and hence, indicate a disease driving role for the SINT1-SMG1-SYT17 axis in diseased humans as well. More importantly, the clearly beneficial effects of SINT1-depletion in iPSC-hCM protecting from structural, metabolic and functional remodeling suggests SINT1 as a novel RNA-target for treatment of heart failure.

Concepts and Evidence of the Invention

Collectively, this invention discloses a novel mode of hypoxia-dependent gene regulation initiated by HIF1α activation of the SINT1 eRNA and its binding to and stimulation of mRNA synthesis of its neighboring gene-promoters SYT17 and SMG1. This mode of gene regulation (as opposed to direct transcriptional activation of SYT17 and SMG1 by HIF1a), provides an effective means of engendering cell-specific hypoxia transcriptional responses and offers a potential mechanistic explanation of at least some of the contextual effects that HIF1α mediates in different tissues and pathologic settings (Vanharanta et al., Nat Med 19, 50 (2013)).

As a mitochondrial-localized member of the calcium-sensing protein family, SYT17 contributes to the regulation of contractility in response to hypoxic stress through re-normalization of expression of a broad range of cell signaling and transcription networks to maintain normal contractility in the face of pathologic insult (FIGS. 3, H, I, and J and FIGS. 11, J and K) (Fernandez-Chacon et al., Nature 410, 41 (2001)). This is phenotypically reflected both in cardiomyocytes in vitro and in vivo in response to TAC (FIG. 6, F to J).

The PI3K-related kinase SMG1 and its downstream phosphorylation target UPF1 represent central components of cell growth control, attributed, in part, to nonsense-mediated decay (NMD), a process dedicated to the control of both the quality and quantity of a large number of mRNAs (Mcllwain et al., Proc Natl Acad Sci USA 107, 12186 (2010)). Although the inventors were unable to detect dramatic shifts in RNA species containing nonsense mutations or premature termination codon in this setting, the inventors did detect shifts in RNAs implicated in metabolic control of cell growth, and in growth pathways (FIG. 12A). Thus, the HIF1α-SINT1 axis may mediate tissue-specific changes in both calcium regulation and growth-dependent gene regulatory processes in response to hypoxia. It is intriguing that embryonic deletion of Smg1 (Mcllwain et al., Proc Natl Acad Sci USA 107, 12186 (2010)) parallels many aspects of Hif1α inactivation in that cardiac growth is suppressed, concomitant to aborted development at looping morphogenesis (Iyer et al., Genes Dev 12, 149 (1998); Krishnan et al., Circ Res 103, 1139 (2008)). Hence, the requirement for HIF1α in development and disease may reflect the need for extensive remodeling of the RNA landscape in cardiac pathology that is known to be coupled to the appearance of transcripts and splice variants of metabolic and sarcomeric proteins not typically expressed in the normal heart (Kong et al., Circ Cardiovasc Genet 3, 138 (2010); Lara-Pezzi et al., J Cardiovasc Transl Res 6, 945 (2013); Wharton et al., J Pharmacol Exp Ther 284, 323 (1998); Agarkova et al., J Biol Chem 275, 10256 (2000)).

Previous studies have attributed the deregulation of cardiac growth and function to the phosphatidylinositol-3 kinase (PI3K) signalling network (Stocker et al., Curr Opin Genet Dev 10, 529 (2000)). PI3Kα activation upon stimulation of receptor tyrosine kinases and G-protein coupled receptors induces pathologic cardiac hypertrophy through induction of protein translation and nucleotide biosynthesis (in part, via mammalian target of rapamycin (mTOR) (Wang et al., Physiology (Bethesda) 21, 362 (2006))), while PI3Kγ activation is linked to contractile dysfunction through inhibited Protein kinase A-cAMP pathway signaling (Crackower et al., Cell 110, 737 (2002); Patrucco et al., Cell 118, 375 (2004)). This intriguing capacity of PI3K signalling to simultaneously regulate two fundamentally critical aspects of disease transition is recapitulated at the phenotypic and gene expression level upon SINT1 inactivation—wherein SINT1-mediated modulation of the SMG1 and SYT17 pro-hypertrophic cluster simultaneously alters the growth and contractile maladaptation associated with disease transition. Strikingly, re-normalisation of SMG1 and SYT17 upregulation induced by TAC, either through Gapmer-mediated SINT1 inactivation, or the dual-targeted inactivation of SMG1 and SYT17 (FIG. 5, C to J and FIG. 6, F to J) rescued and reverted characteristic features of heart disease. While it might be advantageous to transiently modulate PI3Kα and PI3Kγ in heart disease patients, the criticality and ubiquity of PI3K signaling in general cell survival and growth makes it difficult to achieve without off-target side effects. In contrast, the context- and cell-type specificity of SINT1 function serve to circumvent these issues and attenuate and revert disease progression to reduce overall pathology associate mortality and morbidity, an evidenced in our study (FIGS. 5, O, P and S).

Given the correlation between SINT1, SMG1 and SYT17 co-expression in cardiomyopathy in independent human cohorts of HCM and aortic stenosis, it is conceivable to suggest a role of this axis in driving cardiac pathology. Indeed, suppression of stress-induced SINT1 production in vivo in mice resolved established cardiomyopathy through repression of Syt17 and Smg1 transcription, indicating that a tight coupling of enhancer transcription and successive induction of promoters in their vicinity is disease relevant. Both aortic stenosis (as the most prevalent valvular heart disease) and HCM (as the primary cause of sudden cardiac death), represent a large fraction of cardiac disease whose therapy today inefficient to prevent heart failure. Notably, SINT1 (or SMG1 or SYT17) induction was detected neither in human biopsies of dilated cardiomyopathy (DCM) nor in ventricular biopsies of a Muscle LIM protein (Mlp)−/− DCM mouse model (Arber et al., Cell 88, 393 (1997).) (FIG. 14 A-D), suggestive of an etiology- or stage-specific function of SINT1 in cardiac pathology. The translational path for long non-coding RNAs as therapeutic targets is challenging due to, in part, the general rather poor sequence conservation among mammals. It is notable that the SINT1 sequence shares a stretch of 330 bp that displays high sequence conservation among mammals (FIG. 1B), thus offering an opportunity for defining target sequences that work across species for therapeutic development. In contrast to the non-targeted nature of current treatment regimens, the contextual nature of SINT1 expression facilitates specific targeting of the diseased ventricular myocardium. Hence, targeting of context-specific disease-induced eRNAs such as SINT1, represents an attractive avenue for developing targeted therapeutic modalities for the treatment of a variety of pathologies.

Materials and Methods

Animal Breeding and Maintenance

Hif1α fl/fl mice were obtained from Randall S. Johnson (University of California, San Diego, USA) and Gregg L. Semenza (Johns Hopkins University School of Medicine, USA), respectively. Vhl fl/fl mice were kindly provided by Rudolf Jaenisch (Massachusetts Institute of Technology, USA). MLC2v-cre/+ line and Mlp+/+ and Mlp−/− hearts were from Ju Chen (University of California, San Diego, USA). The data presented in this disclosure represents studies with male mice aged from 8-24 weeks old of the C57131/6 background. Mice were randomly assigned to groups, and surgery, AAV injections, Gapmer delivery and echocardiography performed blinded. Animal numbers for experiments were chosen based on expected mortality rates, anticipated phenotype and functional changes of hearts in wild-type mice in response to surgery. Animals were excluded from the study in case of death before the experimental endpoint or based on the evaluation of pain using a standardized score sheet, approved by BVET. All mice were maintained in a specific pathogen-free (SPF) facility at RCHCI and EPIC, ETH Zurich and/or Cardiovascular Assessment Facility (CAF), Department of Medicine, University of Lausanne. Maintenance and animal experimentation were in accordance with the Swiss Federal Veterinary Office (BVET) guidelines.

Human and Mouse Ventricular Biopsies

Left-ventricular samples were obtained from patients with hypertrophic cardiomyopathy aortic stenosis or dilated cardiomyopathy and healthy controls. Clinical and demographical data from patients were previously published (Mirtschink et al., Nature 522,444 (2015)). Biopsies were conducted in compliance with the local ethics committee, and written informed consent was received from all subjects prior to inclusion. Myocardial samples were obtained from patients with severe aortic stenosis undergoing aortic valve replacement and a Morrow resection from the hypertrophied left ventricular septum. Only patients without significant aortic valvular regurgitation and with preserved contractile function were included. Myocardium was also obtained from patients with severe HCM during septal myectomie surgery. The myocardial samples were acquired directly in the operating room during the surgery and immediately placed in precooled cardioplegic solution (110 mM NaCl, 16 mM KCl, 16 mM MgCl₂, 16 mM NiPSC-hCMO₃, 1.2 mM CaCl₂), 11 mM glucose). Samples were frozen (−80° C.) immediately in the surgery room.

Transaortic Banding

16-20 week old mice were subjected to transaortic banding (TAC) through constriction of the aortic arch (between the innominate artery and the left carotid artery) as described (Kassiri et al., Circ Res 97, 380 (2005)). The mice were monitored regularly and their heart functions were determined by echocardiography. SINT1-targeting Gapmers (Exiqon) were generated, stored and used as recommended by the manufacturer. Gapmers were used at a dose of 10 mg/kg or 5 mg/kg as indicated in FIG. 4A.

1 Kidney 1 Clip Surgery

Under inhalation anesthesia by isoflurane a clip was placed around the left renal artery of 6-8 weeks old mice in order to reduce the renal blood flow, whereas the right kidney was removed as previously described (Krishnan et al., Cell Metab 9, 512 (2009)). The sham procedure, including the entire surgery with the exception of artery clipping, was applied in control mice. Postoperative analgesia was provided by subcutaneous application of Temgesic (Buprenorphin 0.1 mg/kg) directly after surgery as well as every 6-8 hours for 2 consecutive days after surgery. Gapmers were used at a dose of 10 mg/kg or 5 mg/kg as indicated in FIG. 4G.

In Vivo Transthoracic Ultrasound Imaging

Transthoracic echocardiography is performed in 2D- and M mode in the parasternal long-axis view using the MS400 (18-38 MHz) probe from Vevo2100 color Doppler ultrasound machine (VisualSonics) as previously described (Ounzain et al., J Mol Cell Cardiol 76, 55 (2014)).

Direct Arterial Blood Pressure Measurement

By a small incision in the carotid artery a heparinized catheter was introduced under inhalation anesthesia by isoflurane as described previously (Krege et al., Hypertension 25, 1111 (1995)). The free end of the catheter was externalized at the neck of the animal. The supply of anesthetic gas was stopped and the animal was returned to its cage placed on a heating surface for complete wake. 3-4 hours after awakening of the animal, the catheter was connected to a pressure sensor. The blood pressure was monitored for about 1 hour in awaked unconstrained animals.

Isolation and Maintenance of Primary Neonatal Mouse Cardiomyocytes

NMC were isolated as described previously (Krishnan et al., Cell Metab 9, 512 (2009)). Cultured cardiomyocytes were treated with phenylepinephrine at a concentration of 100 μM for 48 h, isoproterenol at 10 μM for 48 h and Dimethyloxaloylglycine (DMOG) at 100 μM for 24 h. Gapmers were added to cardiomyocytes at a concentration of 0.5 μM using the Accell siRNA delivery media (Dharmacon) according to the manufacturer's instructions.

Human Cardiomyocyte Culture

Human iPSC derived cardiomyocytes were thawed and cultured as recommended by the manufacturer (Cellular Dynamics International). Cells were transduced with lentiviruses and/or treated with Gapmers at a concentration of 0.5 μM 7-10 days after thawing and harvested at day 10-12.

Lentivirus Production and Transduction

Lentiviruses were generated in HEK-293T cells, purchased from ATCC and regularly checked for the presence of mycoplasma contaminants using a PCR-based detection kit (Sciencell). NMC were transduced as previously described (Mirtschink et al., Nature 522, 444 (2015)).

Lentiviral and Plasmid Expression Constructs

Lentiviral shRNAs were purchased from Sigma or custom designed using BLOCK-iT™ RNAiDesigner (Life Technologies) software and synthesized by Sigma. The following shRNAs in the lentiviral pLKO.1 vector from Sigma as part of their TRC library were purchased: Smg1 (shSmg1, TRCN0000088685) and Syt17 (shSyt17, TRCN0000173230), Hif1α (shHif1α, TRCN0000232220). SHC002 (Sigma) was used as a non-targeting shRNA. The following custom designed shRNAs in the pLKO.1 construct against SINT1 were used: shSINT1 #1, sense (5′-3′) (SEQ ID NO 002) (the sequences give the coding strand DNA sequences encoding the shRNA; the shRNA actually employed is the reverse complementary strand to SEQ 2/3):

### CCGGGGCACATGCGCCACTTAATCACTCGAGTGATTAAGTGGCGCATGTGCTTTTTG

and shSINT1 #3, sense (5′-3′) (SEQ ID NO 003):

### CCGGGGTACATTGACCTGTACTTCCCTCGAGGGAAGTACAGGTCAATGTACCTTTTTG.

The HIF1αΔODD expression construct was generated as described (Huang et al., Proc Natl Acad Sci USA 95, 7987 (1998).) and cloned into pLKO.1-CMV for lentiviral generation (Troilo et al., EMBO Rep 15, 77 (2014)). The pcDNA3 HA-Smg1 expression construct was kindly provided by Oliver Mühlemann (University of Bern, Switzerland).

To generate the AN-SRF fusion protein, the CAGGS promoter followed by AN was subcloned into the pV5-AviC vector between the MluI and SalI restriction sites. Afterwards, SRF was subcloned into the pV5-AN-AviC vector between the XhoI restriction site. SINT1 was amplified from mouse genomic DNA and cloned into the pcDNA3.1 vector between the HindIII and BamHI restriction sites. Subsequently, the 5× BoxB cassette was subcloned from pR6K BoxB vector into pcDNA3.1 vector between the BamHI restriction site. Sequence integrity of the cloned region was verified by sequencing and BLAST alignment (http://www.ncbi.nlm.nih.gov/blast).

Antisense Oligonucleotide Synthesis

Synthesis and purification of Gapmers were performed by Exiqon. As a standard Gapmers were purified and analyzed using anion-exchange HPLC, desalted and lyophilized as a sodium salt. Compound identity was confirmed by ESI-MS at a purity of >85%. The Gapmers contain phosphorothioate backbone modifications and proprietary modifications within the sequence, which differ between the in vivo and in vitro versions of the Gapmers.

Scrambled Ctrl Gapmer (scrGM, in vivo;
SEQ ID NO 004):
5′-TCATACTATATGACAG-3′,
SINT1 #1 Gapmer (SINT1GM1, in vivo;
SEQ ID NO 005):
5′-TGCTTGAAAGTGATGA-3′,
SINT1 Gapmer #3 (SINT1GM3, in vivo;
SEQ ID NO 006):
5′-GTAGAAAGTGGCTAGA-3′.
Scrambled Ctrl Gapmer (scrGM, in vitro;
SEQ ID NO 007):
5′-AACACGTCTATACGC-3′;
SINT1 Gapmer #1 (SINT1GM1, in vitro;
SEQ ID NO 008):
5′-TGCTTGAAAGTGATGA-3′;
SINT1 Gapmer #3 (SINT1GM3, in vitro;
SEQ ID NO 009):
5′-GTAGAAAGTGGCTAGA-3′.

Transient Transfection

293T cells grown in 12-well plates were transfected with 0.8 μg of the AN-SRF fusion construct and 0.8 μg of the SINT1-BoxB vector using Lipofectamine 2000 as recommended by the manufacturer. The medium was changed to DMEM containing 0.5% FCS 4 h after transfection and gene expression was analysed 48 h after transfection.

In Vitro Metabolic Measurements

Cellular oxygen consumption and extracellular pH, as readouts for oxidative metabolism and glycolysis, were measured using the Seahorse Bioscience XF24 or XFe96 Flux Analyzer at 37° C. with correction for positional temperature variations adjusted from four empty wells evenly distributed within the plate. NMC were seeded at 5×10⁴ cells/well 3-5 days prior to the analysis.

In Vitro Contractility Assay

Assays were performed on neonatal mouse and human iPSC derived cardiomyocytes and analyzed with the IonOptix Cell Analyzer System. Cardiomyocytes were placed in a chamber mounted on the stage of an inverted microscope and perfused with a modified tyrode buffer (137 mM NaCl, 5 mM KCl, 15 mM Glucose, 1.3 mM MgSO₄, 1.2 mM NaH₂PO₄, 20 mM HEPES, 1 mM CaCl₂), pH 7.4) and field stimulated at a frequency of 1 Hz. Contractility was recorded and analyzed using the IonWizard software.

Histological Analysis

Hearts were embedded in OCT and sectioned at 10 μm. The sections were fixed for 10 min with 4% PFA/PBS and after 2×2 min PBS washes, the sections were blocked for 1 h with 2% HS/PBS. The sections were permeabilised for 10 min with 0.2% Triton X-100/PBS. After 3×5 min PBS washes, the sections were incubated with the primary antibody diluted in 2% HS/0.025% Triton X-100/overnight in a humidified chamber at 4° C. After 3×10 min PBS washes the secondary antibody was incubated overnight in a humidified chamber at 4° C. After 5×10 min PBS wash, the sections were mounted using ProLong gold Antifade. The sections were stained with laminin (1:200) to visualize the cell outline and imaged using the Leica sp8 confocal microscope. The cell cross-sectional area was quantified using Image J.

Immunocytochemistry and Cell Size Quantification

Immunofluorescent staining was performed as described previously (Krishnan et al., Cell Metab 9, 512 (2009)). Pictures of all channels were taken using a 20× magnification. Using the software Cell Profiler, cardiomyocytes were identified using the cell type-specific antibody and cell area was quantified. Multi-nucleated cells were counted manually and average area per cell was corrected taking into account the number of multi-nucleated cells.

RNA Fish

5×10⁴ NMCs were seeded on poly-L-lysine coated 12 mm cover glasses. RNA FISH was performed using the QuantiGene® ViewRNA ISH Cell Assay Kit as recommended by the manufacturer Affymetrix. The following RNA FISH probes were used: Smg1 (VB4-15594), Syt17 (VB6-15595) and SINT1 (custom synthesis). Samples were imaged using the Leica sp8 confocal microscope.

[³H]Leucine Incorporation Assay

[³H]leucine incorporation was quantified in order to assess protein synthesis in NMC as previously described (Mirtschink et al., Nature 522, 444 (2015)). Radioactive labeled maintenance medium containing 0.5 μCi/ml [³H]leucine was added 3 days after NMC isolation and 4×10⁵ cells per 3 cm dish labeled and incubated for 20 hours. On day three cells were collected by trypsinization. Incorporated radioactivity was normalized to absolute cell number.

Antibodies and Fluorescent Reagents

Antibody against sarcomeric α-actinin (A7811) was from Sigma. The Smg1 antibodies used for immunoblotting (sc-135563) and immunocytochemistry (HPA073972) were from Santa Cruz and Sigma, respectively. The antibody against Syt17 (15413-1-AP) was from Proteintech. Upf1 antibody (07-1014) and phospho-Upf1 (Ser1127, 07-1016) were from Merck Millipore. Hif1α antibody (H1alpha67) used for ChIP and immunoblotting, pan-Cadherin (ab6528), laminin (ab11575) and Atp5a1 (ab14748) antibodies were from Abcam. H3K4me1 (#39297), H3K4me3 (#39159) and H3K27ac (#39133) antibodies used in ChIP were from Active Motif. Myosin binding protein C antibody was kindly provided by Mathias Gautel (King's College London, UK).

SDS-PAGE and Immunoblotting

Dissected hearts were homogenized by freeze slamming and solubilized in a modified SDS sample buffer sonicated and boiled for 5 minutes. Cultured cardiomyocyte lysates were harvested with the modified SDS sample buffer, sonicated and boiled. Protein extracts were resolved on 6-12% polyacrylamide minigels (BioRad) and transferred overnight onto nitrocellulose membrane (GE Healthcare). Immunodetection and visualization of signals by chemiluminescence was carried out as described (Hirschy et al., Dev Biol 289, 430 (2006)).

Luciferase Promoter Assays

1.5 kb of the SINT1 promoter was amplified from mouse BAC genomic DNA and cloned into the pGL3 luciferase reporter vector (Stratagene). The HRE-mutant was generated by recombinant PCR (Casonato et al., J Lab Clin Med 144, 254 (2004); Elion et al., Curr Protoc Mol Biol Chapter 3, Unit 3 17 (2007)). Sequence integrity of the respective wildtype and mutant promoters were verified by sequencing and BLAST alignment (http://www.ncbi.nlm.nih.gov/blast). The reporter assay was performed by transient co-transfection of the appropriate luciferase reporter, pSV-β-galactosidase (Promega), in the presence of HIF1αΔODD. Luciferase and β-galactosidase activity was measured with the Luciferase Assay System kit (Promega) as recommended by the manufacturer and analyzed on the FLUOstar Omega (BMG Labtech). Mouse cardiomyocytes were transfected with Trogene or Lipofectamine 2000 (Life Technologies) as recommended by the manufacturer.

For enhancer activity determination, a 2.9 kb fragment containing SINT1 and additional flanking sequences was amplified from mouse BAC genomic DNA and cloned into the pGL3 luciferase enhancer reporter vector (Stratagene) as previously described (Lam et al., Nature 498, 511 (2013)). A 3 kb control genomic fragment encompassing the peroxisome proliferator activated receptor γ (Pparγ promoter void of enhancer features was amplified and cloned in a similar manner. Sequence integrity of the amplified region was verified by sequencing and BLAST alignment (http://www.ncbi.nlm.nih.gov/blast).

Quantitative RT-PCR

RNA was isolated and genomic DNA removed with the RNeasy Plus Kit (Qiagen) and cDNA generated using Superscript II (Invitrogen) as recommended by the manufacturer. qPCR reactions were setup as recommended by the manufacturer (Biorad) and analyzed on the PikoReal Real-Time PCR System (Thermo Scientific). Ct values were normalized to the housekeeping gene Hprt1. The following qPCR primers were used:

TABLE 1
qPCR primers for mouse sequences

| Target | forward primer | Reverse primer |
|---|---|---|
| Hprt1 | GGGGCTGTACTGCTTAACCA | TGCTCATCAGTTGCCACTTC |
| | G | SEQ ID NO 011 |
| | SEQ ID NO 010 | |
| SINT1 | ACTGAGACCCGAGGCAGTAA | GTCTTCAAGCCTGCCTTCAC |
| | SEQ ID NO 012 | SEQ ID NO 013 |
| Smg1 | AGGGTGGCTACAGTGTCAAT | CCCAACGACTTCCGACCATA |
| | G | SEQ ID NO 015 |
| | SEQ ID NO 014 | |
| Syt17 | ACGCTCGTCCTCAGACACA | CTCAACGGGTTTGATGTCGA |
| | SEQ ID NO 016 | T |
| | | SEQ ID NO 017 |
| Nppa | GTGCGGTGTCCAACACAGAT | TCCAATCCTGTCAATCCTAC |
| | SEQ ID NO 018 | CC |
| | | SEQ ID NO 019 |
| Nppb | AGTCCTTCGGTCTCAAGGCA | CCGATCCGGTCTATCTTGTG |
| | SEQ ID NO 020 | C |
| | | SEQ ID NO 021 |
| Atp2a1 | TGTTTGTCCTATTTCGGGGT | AATCCGCACAAGCAGGTCTT |
| | G | C |
| | SEQ ID NO 022 | SEQ ID NO 023 |
| Atp2a2 | GAGAACGCTCACACAAAGAC | CAATTCGTTGGAGCCCCAT |
| | C | SEQ ID NO 025 |
| | SEQ ID NO 024 | |
| Pln | AAAGTGCAATACCTCACTCG | GGCATTTCAATAGTGGAGGC |
| | C | TC |
| | SEQ ID NO 026 | SEQ ID NO 027 |
| Titin | GACACCACAAGGTGCAAAGT | CCCACTGTTCTTGACCGTAT |
| | C | CT |
| | SEQ ID NO 028 | SEQ ID NO 029 |
| Hk2 | TGATCGCCTGCTTATTCACG | AACCGCCTAGAAATCTCCAG |
| | G | A |
| | SEQ ID NO 030 | SEQ ID NO 031 |
| Aldoc | AGAAGGAGTTGTCGGATATT | TTCTCCACCCCAATTTGGCT |
| | GCT | C |
| | SEQ ID NO 032 | SEQ ID NO 033 |
| Slc2a1 | CAGTTCGGCTATAACACTGG | GCCCCCGACAGAGAAGAT |
| | TG | |
| | SEQ ID NO 034 | SEQ ID NO 035 |
| Gapdh | AGGTCGGTGTGAACGGATTT | TGTAGACCATGTAGTTGAGG |
| | G | TCA |
| | SEQ ID NO 036 | SEQ ID NO 037 |
| Pdk4 | AGGGAGGTCGAGCTGTTCTC | GGAGTGTTCACTAAGCGGTC |
| | SEQ ID NO 038 | A |
| | | SEQ ID NO 039 |
| Pparα | AACATCGAGTGTCGAATATG | AGCCGAATAGTTCGCCGAAA |
| | TGG | G |
| | SEQ ID NO 040 | SEQ ID NO 041 |
| Pparγ | GGAAGACCACTCGCATTCCT | GTAATCAGCAACCATTGGGT |
| | T | CA |
| | SEQ ID NO 042 | SEQ ID NO 043 |

TABLE 2
qPCR primers for human sequences

| Target | forward primer | reverse primer |
|---|---|---|
| HPRT1 | CCTGGCGTCGTGATTAGTG | AGACGTTCAGTCCTGTCCAT |
| | AT | AA |
| | SEQ ID NO 044 | SEQ ID NO 045 |
| SINT1 | CCAGAAGTTAACAAAGAAA | CTTCCAAAATAAGGAAATTT |
| | GAGG | GATGC |
| | SEQ ID NO 046 | SEQ ID NO 047 |
| SMG1 | GGTGGCTCGATGTTACCCT | CTGCGTGAGCGAAGGTTTC |
| | C | SEQ ID NO 049 |
| | SEQ ID NO 048 | |
| SYT17 | ATTCCCCGGATGGAAGACG | CGCCAAACTCGATGGGTTTA |
| | SEQ ID NO 050 | ATA |
| | | SEQ ID NO 051 |
| SRF | CCAAGCCGGGTAAGAAGAC | GTCAGCGTGGACAGCTCATA |
| | C | SEQ ID NO 053 |
| | SEQ ID NO 052 | |
| EGR1 | CCACCACGTACTCCTCTGT | GGTTGCTGTCATGTCCGAAA |
| | T | SEQ ID NO 055 |
| | SEQ ID NO 054 | |
| BoxB | GATAGCGGCCGCGCTCGCT | GCATTTGCAATGAAAATAAA |
| | TTC | TG |
| | SEQ ID NO 056 | SEQ ID NO 057 |

ChIP and ChIRP

ChIP assays were performed using material from NMC and the assay performed using the ChIP-IT kit (Active Motif) as recommended by the manufacturer and analyzed by qPCR. For ChIP-seq analyses (Active Motif) hearts were removed from the mice, snap frozen in liquid N2 and stored at −80° C. For fixation, hearts were cut into small pieces in PBS, 1% formaldehyde and incubated at room temperature for 15 minutes. Fixation was stopped by the addition of 0.125M glycine and tissue pieces were treated with a Tissue-Tearor. Chromatin was isolated by the addition of lysis buffer, followed by disruption with a Dounce homogenizer. Lysates were sonicated and the DNA sheared to an average length of 300-500 bp. Genomic DNA (Input) was prepared by treating aliquots of chromatin with RNase, proteinase K and heat for de-crosslinking, followed by ethanol precipitation. Pellets were resuspended and the resulting DNA was quantified on a NanoDrop spectrophotometer. Extrapolation to the original chromatin volume allowed quantitation of the total chromatin yield. An aliquot of chromatin (30 μg) was pre-cleared with protein A agarose beads (Invitrogen). Genomic DNA regions of interest were isolated using 4 μg of antibody against Hif1α, H3K4me1 and H3K4me3. Complexes were washed, eluted from the beads with SDS buffer, and subjected to RNase and proteinase K treatment. Crosslinks were reversed by incubation overnight at 65° C., and ChIP DNA was purified by phenol-chloroform extraction and ethanol precipitation. qPCR reactions were carried out in triplicate on specific genomic regions using SYBR Green Supermix (Bio-Rad). The resulting signals were normalized for primer efficiency by carrying out qPCR for each primer pair using Input DNA. ChIP and Input DNAs were prepared for amplification by converting overhangs into phosphorylated blunt ends and adding an adenine to the 3′-ends. Illumina genomic adapters were ligated and the sample was size-fractionated (200-300 bp) on a 2% agarose gel. After a final PCR amplification step (18 cycles), the resulting DNA libraries were quantified and sequenced on HiSeq 2500 (50 nucleotide reads, single-end). Reads were aligned to the mouse genome (mm9) using the BWA algorithm. Duplicate reads were removed and only uniquely mapped reads (mapping quality >=25) were used for further analysis. Alignments were extended in silico at their 3′-ends to a length of 150 bp, which is the average genomic fragment length in the size-selected library and assigned to 32-nt bins along the genome. The resulting histograms (genomic “signal maps”) were stored in BAR and bigWig files. Peak locations were determined using the MACS algorithm (v 1.4.2) with a cutoff of p-value=1e⁻⁷. Signal maps and peak locations were used as input data to Active Motifs proprietary analysis program, which creates Excel tables containing detailed information on sample comparison, peak metrics, peak locations and gene annotations. In silico promoter analyses and alignments were performed using Matlnspector and DiAlignTF (Genomatix). Primer sequences used for SINT1 in the ChIP were 5′-CCACAGAGCAGGAAGCAGAGA-3′ (SEQ ID NO 058) and 5′-GGTTTGAATGCGAAATGTCCTTAC-3′ (SEQ ID NO 059). ChIRP was performed as previously described (Chu et al., Mol Cell 44, 667 (2011)) using biotinylated oligonucleotides from Microsynth AG or Sigma.

RNA Sequencing

The twelve paired-end libraries (three replicates for each four samples) were processed by using the Trimmomatic v0.36 (Bolger et al., Bioinformatics 30, 2114 (2014)) software. All Illumina standard adapter and primer sequences were trimmed and read length cutoff of 60 bases and a window based quality filtering (window length: 5base; phred quality score cutoff: 20) was applied. Filtered reads were mapped to the genome of Rattus norvegicus (Gen Bank assembly accession: GCA_000001895.4) by using the STAR v2.5.3a (Dobin et al., Bioinformatics 29, 15 (2013)) RNA-seq aligner. The generated binary alignment map (bam) files were processed by using the featureCounts v1.5.0-p3 (Liao et al., Bioinformatics 30, 923 (2014)) software for generating a count matrix. Differential gene expression analysis was carried out by using the DeSeq2 (Love et al., Genome Biol 15, 550 (2014)) software, by importing the count matrix. Differential gene expression analyses were performed by taking normoxic libraries as control to other treated libraries. Differentially expressed genes were filtered by taking a p-value cutoff of 0.05 of FDR (false discovery rate) test. KEGG enrichment of the differentially expressed genes were carried out by using p-value <0.05 by using the clusterProfiler (Yu et al., OMICS 16, 284 (2012)) package in R (https://www.r-project.org/). After performing the trimming and filtering step, on an average 86.44% sequences could be retrieved from the twelve libraries. For generating an abundance count matrix all twelve libraries were mapped, by using STAR aligner, on the Rattus norvegicus genome. STAR mapping showed an average of 88.80% of reads from the twelve libraries could be mapped. The abundance count matrix was imported to the DeSeq2 software for calculating the fold change values with respect to the normoxic libraries. In total three replicates for each sample was used for evaluating the differential gene expression.

Statistical Analysis

For statistical analyses, unpaired, two-tailed Student's t-tests (Excel) or one-way ANOVA analyses followed by a Dunnet's multiple comparison post-test were used as indicated in the respective figure legends. No statistical methods were used to predetermine sample size.

Validation of Antisense Oligonucleotides Directed Against Human SINT1

iPSC-hCM expressing ectopic HIF1αΔODD were treated with the respective gapmers of table 3 and tested for SINT1 knockdown by qPCR (FIG. 16).

TABLE 3
Gapmer sequences directed against humanSINT1

| Name | Sequence 5′-3′ | Name | Sequence 5′-3′ |
|---|---|---|---|
| hSINT1 | TGCTTGAAAGTGATGA | hSINT1 | GTAGAAAGTGGCTAGA |
| #1 | SEQ ID NO 005 | #3 | SEQ ID NO 006 |
| hSINT1 | AGAATCTATCCGAATG | hSINT1 | ATCTTATCAGATTCCT |
| #2 | SEQ ID NO 060 | #77 | GCT |
| | | | SEQ ID NO 131 |
| hSINT1 | TTAAAGTGTTTCCTCC | hSINT1 | AACATTCGGATAGATT |
| #4 | SEQ ID NO 061 | #78 | CTG |
| | | | SEQ ID NO 132 |
| hSINT1 | ACTTTCTAAATTGCTC | hSINT1 | TCTTATCAGATTCCTG |
| #6 | SEQ ID NO 062 | #79 | CTG |
| | | | SEQ ID NO 133 |
| hSINT1 | ATCTATCCGAATGTTA | hSINT1 | AGTAAATTCAATGAGC |
| #7 | SEQ ID NO 063 | #80 | ACC |
| | | | SEQ ID NO 134 |
| hSINT1 | TCTAAATTGCTCAGCA | hSINT1 | AAACGCAACAGAAATG |
| #8 | SEQ ID NO 064 | #81 | TCA |
| | | | SEQ ID NO 135 |
| hSINT1 | TACTCACTCTCTCTAG | hSINT1 | TAAGGGAGGAGGAAAC |
| #9 | SEQ ID NO 065 | #82 | ACT |
| | | | SEQ ID NO 136 |
| hSINT1 | TCCCAGGCCATTCTC | hSINT1 | TTGTTTGTTTTTCGTG |
| #10 | SEQ ID NO 066 | #83 | AGA |
| | | | SEQ ID NO 137 |
| hSINT1 | ACAGAATCTATCCGA | hSINT1 | TGTTTGTTTTTCGTGA |
| #11 | SEQ ID NO 067 | #84 | GAC |
| | | | SEQ ID NO 138 |
| hSINT1 | TAATTAAAGTGTTTCC | hSINT1 | AATTTTTTCTTTTTAC |
| #12 | SEQ ID NO 068 | #85 | TCT |
| | | | SEQ ID NO 139 |
| hSINT1 | TACTTTTCCCAGGCCA | hSINT1 | ATTCAATGAGCACCTC |
| #15 | SEQ ID NO 069 | #86 | CAG |
| | | | SEQ ID NO 140 |
| hSINT1 | TTAGAAAGTGAAACCA | hSINT1 | AACCAGCTATTTCTGG |
| #16 | AGC | #87 | CTC |
| | SEQ ID NO 070 | | SEQ ID NO 141 |
| hSINT1 | TTTTTACTCTTTTCTC | hSINT1 | AATAAGAACCCCAGAT |
| #17 | AGC | #88 | TGC |
| | SEQ ID NO 071 | | SEQ ID NO 142 |
| hSINT1 | TTAACATTCGGATAGA | hSINT1 | TTTTTCTGATTCTCTG |
| #18 | TTC | #89 | GTA |
| | SEQ ID NO 072 | | SEQ ID NO 143 |
| hSINT1 | TTACTCTTTTCTCAGC | hSINT1 | CAATTTAGAAAGTGAA |
| #19 | ATC | #90 | ACC |
| | SEQ ID NO 073 | | SEQ ID NO 144 |
| hSINT1 | TCATATTATTAATTAC | hSINT1 | ACACTTTAATTAACAT |
| #20 | TGC | #91 | TCG |
| | SEQ ID NO 074 | | SEQ ID NO 145 |
| hSINT1 | TTACTGCATTGGTTCA | hSINT1 | ACACATTCATCTGGAC |
| #21 | TGG | #92 | TTC |
| | SEQ ID NO 075 | | SEQ ID NO 146 |
| hSINT1 | TTTCTCAGCATCTTAT | hSINT1 | TGTTTTTCGTGAGACG |
| #22 | CAG | #93 | GAG |
| | SEQ ID NO 076 | | SEQ ID NO 147 |
| hSINT1 | TTAATTACTGCATTGG | hSINT1 | TGTTTGTTTGTTTTTC |
| #23 | TTC | #94 | GTG |
| | SEQ ID NO 077 | | SEQ ID NO 148 |
| hSINT1 | TTATTAATTACTGCAT | hSINT1 | AAAGTAAATTCAATGA |
| #24 | TGG | #95 | GCA |
| | SEQ ID NO 078 | | SEQ ID NO 149 |
| hSINT1 | TAAACCAGCTATTTCT | hSINT1 | AGTCAGAATTGGAATA |
| #25 | GGC | #96 | CAC |
| | SEQ ID NO 079 | | SEQ ID NO 150 |
| hSINT1 | TTTAGAAAGTGAAACC | hSINT1 | CACTTTAATTAACATT |
| #26 | AAG | #97 | CGG |
| | SEQ ID NO 080 | | SEQ ID NO 151 |
| hSINT1 | AAAAGTAAATTCAATG | hSINT1 | AGGAAACACTTTAATT |
| #27 | AGC | #98 | AAC |
| | SEQ ID NO 081 | | SEQ ID NO 152 |
| hSINT1 | TTCTCAGCATCTTATC | hSINT1 | AATGTCATTTTCCTTG |
| #28 | AGA | #99 | GGT |
| | SEQ ID NO 082 | | SEQ ID NO 153 |
| hSINT1 | TATCAGATTCCTGCTG | hSINT1 | AGAATTGGAATACACA |
| #29 | AGC | #100 | TTC |
| | SEQ ID NO 083 | | SEQ ID NO 154 |
| hSINT1 | TTTCTGATTCTCTGGT | hSINT1 | AATTAACATTCGGATA |
| #30 | AAC | #101 | GAT |
| | SEQ ID NO 084 | | SEQ ID NO 155 |
| hSINT1 | TTTTCTTTTTACTCTT | hSINT1 | AATTGGAATACACATT |
| #31 | TTC | #102 | CAT |
| | SEQ ID NO 085 | | SEQ ID NO 156 |
| hSINT1 | TTAATTAACATTCGGA | hSINT1 | AAGTGAAACCAAGCCT |
| #32 | TAG | #103 | GAG |
| | SEQ ID NO 086 | | SEQ ID NO 157 |
| hSINT1 | TAAATTCAATGAGCAC | hSINT1 | AATTTAGAAAGTGAAA |
| #33 | CTC | #104 | CCA |
| | SEQ ID NO 087 | | SEQ ID NO 158 |
| hSINT1 | TAACATTCGGATAGAT | hSINT1 | ATAGATTCTGTTCCTC |
| #34 | TCT | #105 | ATA |
| | SEQ ID NO 088 | | SEQ ID NO 159 |
| hSINT1 | AACAGAAATGTCATTT | hSINT1 | AAAGTGAAACCAAGCC |
| #35 | TCC | #106 | TGA |
| | SEQ ID NO 089 | | SEQ ID NO 160 |
| hSINT1 | TAACAGAGACTGTTTT | hSINT1 | CAACAGAAATGTCATT |
| #36 | CCC | #107 | TTC |
| | SEQ ID NO 090 | | SEQ ID NO 161 |
| hSINT1 | TTTGTTTTTCGTGAGA | hSINT1 | CTTTTCTCAGCATCTT |
| #37 | CGG | #108 | ATC |
| | SEQ ID NO 091 | | SEQ ID NO 162 |
| hSINT1 | TTTTCTCAGCATCTTA | hSINT1 | ATTTCCCTGAACAGGT |
| #38 | TCA | #109 | AGA |
| | SEQ ID NO 092 | | SEQ ID NO 163 |
| hSINT1 | ATTGGAATACACATTC | hSINT1 | AGAAATGTCATTTTCC |
| #39 | ATC | #110 | TTG |
| | SEQ ID NO 093 | | SEQ ID NO 164 |
| hSINT1 | TTGTTTTTCTGATTCT | hSINT1 | AACAGAGACTGTTTTC |
| #40 | CTG | #111 | CCG |
| | SEQ ID NO 094 | | SEQ ID NO 165 |
| hSINT1 | TATTAATTACTGCATT | hSINT1 | AAACCAAGCCTGAGTC |
| #41 | GGT | #112 | AGA |
| | SEQ ID NO 095 | | SEQ ID NO 166 |
| hSINT1 | TTATCAGATTCCTGCT | hSINT1 | CTCAGATGATCTGTCT |
| #42 | GAG | #113 | AGG |
| | SEQ ID NO 096 | | SEQ ID NO 167 |
| hSINT1 | AAATTCAATGAGCACC | hSINT1 | AATTCAATGAGCACCT |
| #43 | TCC | #114 | CCA |
| | SEQ ID NO 097 | | SEQ ID NO 168 |
| hSINT1 | TTTTGTTTTTCTGATT | hSINT1 | ATCAGATTCCTGCTGA |
| #44 | CTC | #115 | GCA |
| | SEQ ID NO 098 | | SEQ ID NO 169 |
| hSINT1 | ATCTGGACTTCGTATT | hSINT1 | ATGATCTGTCTAGGGG |
| #45 | TCC | #116 | AGC |
| | SEQ ID NO 099 | | SEQ ID NO 170 |
| hSINT1 | TTTTACTCTTTTCTCA | hSINT1 | AATTACTGCATTGGTT |
| #46 | GCA | #117 | CAT |
| | SEQ ID NO 100 | | SEQ ID NO 171 |
| hSINT1 | ATACACATTCATCTGG | hSINT1 | ATGATGGATTCTCCTG |
| #47 | ACT | #118 | CCT |
| | SEQ ID NO 101 | | SEQ ID NO 172 |
| hSINT1 | TTTTTCGTGAGACGGA | hSINT1 | ACTTCGTATTTCCCTG |
| #48 | GTC | #119 | AAC |
| | SEQ ID NO 102 | | SEQ ID NO 173 |
| hSINT1 | AATACACATTCATCTG | hSINT1 | ACCAAACGCAACAGAA |
| #49 | GAC | #120 | ATG |
| | SEQ ID NO 103 | | SEQ ID NO 174 |
| hSINT1 | ATTCTCTGGTAACAGA | hSINT1 | ACTTTAATTAACATTC |
| #50 | GAC | #121 | GGA |
| | SEQ ID NO 104 | | SEQ ID NO 175 |
| hSINT1 | TTTGTTTGTTTGTTGT | hSINT1 | AGTGAAACCAAGCCTG |
| #51 | TTG | #122 | AGT |
| | SEQ ID NO 105 | | SEQ ID NO 176 |
| hSINT1 | TTTGTTTGTTGTTTGT | hSINT1 | ATTGCAATGGGCCTTC |
| #52 | TTG | #123 | TGA |
| | SEQ ID NO 106 | | SEQ ID NO 177 |
| hSINT1 | TAATTAACATTCGGAT | hSINT1 | GAAATGTCATTTTCCT |
| #53 | AGA | #124 | TGG |
| | SEQ ID NO 107 | | SEQ ID NO 178 |
| hSINT1 | AAGTAAATTCAATGAG | hSINT1 | AATGAGCACCTCCAGG |
| #54 | CAC | #125 | GGG |
| | SEQ ID NO 108 | | SEQ ID NO 179 |
| hSINT1 | TGTTTTTCTGATTCTC | hSINT1 | CTAGAGAGAGTGAGTA |
| #55 | TGG | #126 | AGG |
| | SEQ ID NO 109 | | SEQ ID NO 180 |
| hSINT1 | TTCTGATTCTCTGGTA | hSINT1 | ATAAGAACCCCAGATT |
| #56 | ACA | #127 | GCA |
| | SEQ ID NO 110 | | SEQ ID NO 181 |
| hSINT1 | ATTCGGATAGATTCTG | hSINT1 | AGATTCTGTTCCTCAT |
| #57 | TTC | #128 | ATT |
| | SEQ ID NO 111 | | SEQ ID NO 182 |
| hSINT1 | TTTACTCTTTTCTCAG | hSINT1 | ACTGTTTTCCCGCCCA |
| #58 | CAT | #129 | ACC |
| | SEQ ID NO 112 | | SEQ ID NO 183 |
| hSINT1 | TTTTTTGTTTGTTTGT | hSINT1 | AAGAACCCCAGATTGC |
| #59 | TGT | #130 | AAT |
| | SEQ ID NO 113 | | SEQ ID NO 184 |
| hSINT1 | ATTATTAATTACTGCA | hSINT1 | AGAGAGTGAGTAAGGG |
| #60 | TTG | #131 | AGG |
| | SEQ ID NO 114 | | SEQ ID NO 185 |
| hSINT1 | TTTGTTTGTTTGTTTT | hSINT1 | AGAACCCCAGATTGCA |
| #61 | TCG | #132 | ATG |
| | SEQ ID NO 115 | | SEQ ID NO 186 |
| hSINT1 | TAATTACTGCATTGGT | hSINT1 | AGAGACTGTTTTCCCG |
| #62 | TCA | #133 | CCC |
| | SEQ ID NO 116 | | SEQ ID NO 187 |
| hSINT1 | ATTGGTTCATGGTAAT | hSINT1 | AGATGATCTGTCTAGG |
| #63 | CCC | #134 | GGA |
| | SEQ ID NO 117 | | SEQ ID NO 188 |
| hSINT1 | TCGTATTTCCCTGAAC | hSINT1 | AGAATGGCCTGGGAAA |
| #64 | AGG | #135 | AGT |
| | SEQ ID NO 118 | | SEQ ID NO 189 |
| hSINT1 | ATTACTGCATTGGTTC | hSINT1 | AGAGAGAGTGAGTAAG |
| #65 | ATG | #136 | GGA |
| | SEQ ID NO 119 | | SEQ ID NO 190 |
| hSINT1 | AACAGGTAGAATAAGA | hSINT1 | AGGTAGAATAAGAACC |
| #66 | ACC | #137 | CCA |
| | SEQ ID NO 120 | | SEQ ID NO 191 |
| hSINT1 | TTTTCGTGAGACGGAG | hSINT1 | AGTAAGGGAGGAGGAA |
| #67 | TCT | #138 | ACA |
| | SEQ ID NO 121 | | SEQ ID NO 192 |
| hSINT1 | TATTTCCCTGAACAGG | hSINT1 | GGGAGAGCCAGAAATA |
| #68 | TAG | #139 | GCTGGTTTA |
| | SEQ ID NO 122 | | SEQ ID NO 193 |
| hSINT1 | TTTTTTTGTTTGTTTG | hSINT1 | CCTCCTGGGATTACCA |
| #69 | TTG | #140 | TGAACCAAT |
| | SEQ ID NO 123 | | SEQ ID NO 194 |
| hSINT1 | ATTCTCCTGCCTCAGC | hSINT1 | CCTGGGATTACCATGA |
| #70 | TCC | #141 | ACCAATGCA |
| | SEQ ID NO 124 | | SEQ ID NO 195 |
| hSINT1 | TATTTCTGGCTCTCCC | hSINT1 | GGGATTACCATGAACC |
| #71 | CGC | #142 | AATGCAGTA |
| | SEQ ID NO 125 | | SEQ ID NO 196 |
| hSINT1 | TTTGTTTGTTTTTCGT | hSINT1 | GGATTACCATGAACCA |
| #72 | GAG | #143 | ATGCAGTAA |
| | SEQ ID NO 126 | | SEQ ID NO 197 |
| hSINT1 | TTTAATTAACATTCGG | hSINT1 | GATTACCATGAACCAA |
| #73 | ATA | #144 | TGCAGTAAT |
| | SEQ ID NO 127 | | SEQ ID NO 198 |
| hSINT1 | TCATGGTAATCCCAGG | hSINT1 | CCTCCTCCCTTACTCA |
| #74 | AGG | #145 | CTCTCTCTA |
| | SEQ ID NO 128 | | SEQ ID NO 199 |
| hSINT1 | TTGTTTTTCGTGAGAC | hSINT1 | CCTGGAGGTGCTCATT |
| #75 | GGA | #146 | GAATTTACT |
| | SEQ ID NO 129 | | SEQ ID NO 200 |
| hSINT1 | AAATGTCATTTTCCTT | hSINT1 | TGGAGGTGCTCATTGA |
| #76 | GGG | #147 | ATTTACTTT |
| | SEQ ID NO 130 | | SEQ ID NO 201 |
| | | hSINT1 | AGTCTCTGTTACCAGA |
| | | #148 | GAATCAGAA |
| | | | SEQ ID NO 202 |

The positions of any sequence in Table 3 can be chosen from LNA, PNA, DNA. In certain embodiments, central (8 to 12, particularly 10) positions are DNA; flanking sequences (4-2, respectively) on either side are LNA or PNA. In certain embodiments, the LNA sequences are linked by phosphothioate ester bonds.

TABLE 4
Gapmer sequences directed against humanSINT1

| Name | Sequence 5′-3′ | Name | Sequence 5′-3′ |
|---|---|---|---|
| hSINT1 | TGCTTGAAAGTGATGA | hSINT1 | GTAGAAAGTGGCTAGA |
| #1 | SEQ ID NO 008 | #3 | SEQ ID NO 009 |
| hSINT1 | AGAATCTATCCGAATG | hSINT1 | ATCTTATCAGATTCCT |
| #2 | SEQ ID NO 203 | #77 | GCT |
| | | | SEQ ID NO 222 |
| hSINT1 | TTAAAGTGTTTCCTCC | hSINT1 | AACATTCGGATAGATT |
| #4 | SEQ ID NO 204 | #78 | CTG |
| | | | SEQ ID NO 223 |
| hSINT1 | ACTTTCTAAATTGCTC | hSINT1 | TCTTATCAGATTCCTG |
| #6 | SEQ ID NO 205 | #79 | CTG |
| | | | SEQ ID NO 224 |
| hSINT1 | ATCTATCCGAATGTTA | hSINT1 | AGTAAATTCAATGAGC |
| #7 | SEQ ID NO 206 | #80 | ACC |
| | | | SEQ ID NO 225 |
| hSINT1 | TCTAAATTGCTCAGCA | hSINT1 | AAACGCAACAGAAATG |
| #8 | SEQ ID NO 207 | #81 | TCA |
| | | | SEQ ID NO 226 |
| hSINT1 | TACTCACTCTCTCTAG | hSINT1 | TAAGGGAGGAGGAAAC |
| #9 | SEQ ID NO 208 | #82 | ACT |
| | | | SEQ ID NO 227 |
| hSINT1 | TCCCAGGCCATTCTC | hSINT1 | TTGTTTGTTTTTCGTG |
| #10 | SEQ ID NO 209 | #83 | AGA |
| | | | SEQ ID NO 228 |
| hSINT1 | ACAGAATCTATCCGA | hSINT1 | TGTTTGTTTTTCGTGA |
| #11 | SEQ ID NO 210 | #84 | GAC |
| | | | SEQ ID NO 229 |
| hSINT1 | TAATTAAAGTGTTTCC | hSINT1 | AATTTTTTCTTTTTAC |
| #12 | SEQ ID NO 211 | #85 | TCT |
| | | | SEQ ID NO 230 |
| hSINT1 | TACTTTTCCCAGGCCA | hSINT1 | ATTCAATGAGCACCTC |
| #15 | SEQ ID NO 212 | #86 | CAG |
| | | | SEQ ID NO 231 |
| hSINT1 | TTAGAAAGTGAAACCA | hSINT1 | AACCAGCTATTTCTGG |
| #16 | AGC | #87 | CTC |
| | SEQ ID NO 213 | | SEQ ID NO 232 |
| hSINT1 | TTTTTACTCTTTTCTC | hSINT1 | AATAAGAACCCCAGAT |
| #17 | AGC | #88 | TGC |
| | SEQ ID NO 214 | | SEQ ID NO 233 |
| hSINT1 | TTAACATTCGGATAGA | hSINT1 | TTTTTCTGATTCTCTG |
| #18 | TTC | #89 | GTA |
| | SEQ ID NO 215 | | SEQ ID NO 234 |
| hSINT1 | TTACTCTTTTCTCAGC | hSINT1 | CAATTTAGAAAGTGAA |
| #19 | ATC | #90 | ACC |
| | SEQ ID NO 216 | | SEQ ID NO 235 |
| hSINT1 | TCATATTATTAATTAC | hSINT1 | ACACTTTAATTAACAT |
| #20 | TGC | #91 | TCG |
| | SEQ ID NO 217 | | SEQ ID NO 236 |
| hSINT1 | TTACTGCATTGGTTCA | hSINT1 | ACACATTCATCTGGAC |
| #21 | TGG | #92 | TTC |
| | SEQ ID NO 218 | | SEQ ID NO 237 |
| hSINT1 | TTTCTCAGCATCTTAT | hSINT1 | TGTTTTTCGTGAGACG |
| #22 | CAG | #93 | GAG |
| | SEQ ID NO 219 | | SEQ ID NO 238 |
| hSINT1 | TTAATTACTGCATTGG | hSINT1 | TGTTTGTTTGTTTTTC |
| #23 | TTC | #94 | GTG |
| | SEQ ID NO 220 | | SEQ ID NO 239 |
| hSINT1 | TTATTAATTACTGCAT | hSINT1 | AAAGTAAATTCAATGA |
| #24 | TGG | #95 | GCA |
| | SEQ ID NO 221 | | SEQ ID NO 240 |
| hSINT1 | TAAACCAGCTATTTCT | hSINT1 | AGTCAGAATTGGAATA |
| #25 | GGC | #96 | CAC |
| | SEQ ID NO 241 | | SEQ ID NO 264 |
| hSINT1 | TTTAGAAAGTGAAACC | hSINT1 | CACTTTAATTAACATT |
| #26 | AAG | #97 | CGG |
| | SEQ ID NO 242 | | SEQ ID NO 265 |
| hSINT1 | AAAAGTAAATTCAATG | hSINT1 | AGGAAACACTTTAATT |
| #27 | AGC | #98 | AAC |
| | SEQ ID NO 243 | | SEQ ID NO 266 |
| hSINT1 | TTCTCAGCATCTTATC | hSINT1 | AATGTCATTTTCCTTG |
| #28 | AGA | #99 | GGT |
| | SEQ ID NO 244 | | SEQ ID NO 267 |
| hSINT1 | TATCAGATTCCTGCTG | hSINT1 | AGAATTGGAATACACA |
| #29 | AGC | #100 | TTC |
| | SEQ ID NO 245 | | SEQ ID NO 268 |
| hSINT1 | TTTCTGATTCTCTGGT | hSINT1 | AATTAACATTCGGATA |
| #30 | AAC | #101 | GAT |
| | SEQ ID NO 246 | | SEQ ID NO 269 |
| hSINT1 | TTTTCTTTTTACTCTT | hSINT1 | AATTGGAATACACATT |
| #31 | TTC | #102 | CAT |
| | SEQ ID NO 247 | | SEQ ID NO 270 |
| hSINT1 | TTAATTAACATTCGGA | hSINT1 | AAGTGAAACCAAGCCT |
| #32 | TAG | #103 | GAG |
| | SEQ ID NO 248 | | SEQ ID NO 271 |
| hSINT1 | TAAATTCAATGAGCAC | hSINT1 | AATTTAGAAAGTGAAA |
| #33 | CTC | #104 | CCA |
| | SEQ ID NO 249 | | SEQ ID NO 272 |
| hSINT1 | TAACATTCGGATAGAT | hSINT1 | ATAGATTCTGTTCCTC |
| #34 | TCT | #105 | ATA |
| | SEQ ID NO 250 | | SEQ ID NO 273 |
| hSINT1 | AACAGAAATGTCATTT | hSINT1 | AAAGTGAAACCAAGCC |
| #35 | TCC | #106 | TGA |
| | SEQ ID NO 251 | | SEQ ID NO 274 |
| hSINT1 | TAACAGAGACTGTTTT | hSINT1 | CAACAGAAATGTCATT |
| #36 | CCC | #107 | TTC |
| | SEQ ID NO 252 | | SEQ ID NO 275 |
| hSINT1 | TTTGTTTTTCGTGAGA | hSINT1 | CTTTTCTCAGCATCTT |
| #37 | CGG | #108 | ATC |
| | SEQ ID NO 253 | | SEQ ID NO 276 |
| hSINT1 | TTTTCTCAGCATCTTA | hSINT1 | ATTTCCCTGAACAGGT |
| #38 | TCA | #109 | AGA |
| | SEQ ID NO 254 | | SEQ ID NO 277 |
| hSINT1 | ATTGGAATACACATTC | hSINT1 | AGAAATGTCATTTTCC |
| #39 | ATC | #110 | TTG |
| | SEQ ID NO 255 | | SEQ ID NO 278 |
| hSINT1 | TTGTTTTTCTGATTCT | hSINT1 | AACAGAGACTGTTTTC |
| #40 | CTG | #111 | CCG |
| | SEQ ID NO 256 | | SEQ ID NO 279 |
| hSINT1 | TATTAATTACTGCATT | hSINT1 | AAACCAAGCCTGAGTC |
| #41 | GGT | #112 | AGA |
| | SEQ ID NO 257 | | SEQ ID NO 280 |
| hSINT1 | TTATCAGATTCCTGCT | hSINT1 | CTCAGATGATCTGTCT |
| #42 | GAG | #113 | AGG |
| | SEQ ID NO 258 | | SEQ ID NO 281 |
| hSINT1 | AAATTCAATGAGCACC | hSINT1 | AATTCAATGAGCACCT |
| #43 | TCC | #114 | CCA |
| | SEQ ID NO 259 | | SEQ ID NO 282 |
| hSINT1 | TTTTGTTTTTCTGATT | hSINT1 | ATCAGATTCCTGCTGA |
| #44 | CTC | #115 | GCA |
| | SEQ ID NO 260 | | SEQ ID NO 283 |
| hSINT1 | ATCTGGACTTCGTATT | hSINT1 | ATGATCTGTCTAGGGG |
| #45 | TCC | #116 | AGC |
| | SEQ ID NO 261 | | SEQ ID NO 284 |
| hSINT1 | TTTTACTCTTTTCTCA | hSINT1 | AATTACTGCATTGGTT |
| #46 | GCA | #117 | CAT |
| | SEQ ID NO 262 | | SEQ ID NO 285 |
| hSINT1 | ATACACATTCATCTGG | hSINT1 | ATGATGGATTCTCCTG |
| #47 | ACT | #118 | CCT |
| | SEQ ID NO 263 | | SEQ ID NO 286 |
| hSINT1 | TTTTTCGTGAGACGGA | hSINT1 | ACTTCGTATTTCCCTG |
| #48 | GTC | #119 | AAC |
| | SEQ ID NO 287 | | SEQ ID NO 310 |
| hSINT1 | AATACACATTCATCTG | hSINT1 | ACCAAACGCAACAGAA |
| #49 | GAC | #120 | ATG |
| | SEQ ID NO 288 | | SEQ ID NO 311 |
| hSINT1 | ATTCTCTGGTAACAGA | hSINT1 | ACTTTAATTAACATTC |
| #50 | GAC | #121 | GGA |
| | SEQ ID NO 289 | | SEQ ID NO 312 |
| hSINT1 | TTTGTTTGTTTGTTGT | hSINT1 | AGTGAAACCAAGCCTG |
| #51 | TTG | #122 | AGT |
| | SEQ ID NO 290 | | SEQ ID NO 313 |
| hSINT1 | TTTGTTTGTTGTTTGT | hSINT1 | ATTGCAATGGGCCTTC |
| #52 | TTG | #123 | TGA |
| | SEQ ID NO 291 | | SEQ ID NO 314 |
| hSINT1 | TAATTAACATTCGGAT | hSINT1 | GAAATGTCATTTTCCT |
| #53 | AGA | #124 | TGG |
| | SEQ ID NO 292 | | SEQ ID NO 315 |
| hSINT1 | AAGTAAATTCAATGAG | hSINT1 | AATGAGCACCTCCAGG |
| #54 | CAC | #125 | GGG |
| | SEQ ID NO 293 | | SEQ ID NO 316 |
| hSINT1 | TGTTTTTCTGATTCTC | hSINT1 | CTAGAGAGAGTGAGTA |
| #55 | TGG | #126 | AGG |
| | SEQ ID NO 294 | | SEQ ID NO 317 |
| hSINT1 | TTCTGATTCTCTGGTA | hSINT1 | ATAAGAACCCCAGATT |
| #56 | ACA | #127 | GCA |
| | SEQ ID NO 295 | | SEQ ID NO 318 |
| hSINT1 | ATTCGGATAGATTCTG | hSINT1 | AGATTCTGTTCCTCAT |
| #57 | TTC | #128 | ATT |
| | SEQ ID NO 296 | | SEQ ID NO 319 |
| hSINT1 | TTTACTCTTTTCTCAG | hSINT1 | ACTGTTTTCCCGCCCA |
| #58 | CAT | #129 | ACC |
| | SEQ ID NO 297 | | SEQ ID NO 320 |
| hSINT1 | TTTTTTGTTTGTTTGT | hSINT1 | AAGAACCCCAGATTGC |
| #59 | TGT | #130 | AAT |
| | SEQ ID NO 298 | | SEQ ID NO 321 |
| hSINT1 | ATTATTAATTACTGCA | hSINT1 | AGAGAGTGAGTAAGGG |
| #60 | TTG | #131 | AGG |
| | SEQ ID NO 299 | | SEQ ID NO 322 |
| hSINT1 | TTTGTTTGTTTGTTTT | hSINT1 | AGAACCCCAGATTGCA |
| #61 | TCG | #132 | ATG |
| | SEQ ID NO 300 | | SEQ ID NO 323 |
| hSINT1 | TAATTACTGCATTGGT | hSINT1 | AGAGACTGTTTTCCCG |
| #62 | TCA | #133 | CCC |
| | SEQ ID NO 301 | | SEQ ID NO 324 |
| hSINT1 | ATTGGTTCATGGTAAT | hSINT1 | AGATGATCTGTCTAGG |
| #63 | CCC | #134 | GGA |
| | SEQ ID NO 302 | | SEQ ID NO 325 |
| hSINT1 | TCGTATTTCCCTGAAC | hSINT1 | AGAATGGCCTGGGAAA |
| #64 | AGG | #135 | AGT |
| | SEQ ID NO 303 | | SEQ ID NO 326 |
| hSINT1 | ATTACTGCATTGGTTC | hSINT1 | AGAGAGAGTGAGTAAG |
| #65 | ATG | #136 | GGA |
| | SEQ ID NO 304 | | SEQ ID NO 327 |
| hSINT1 | AACAGGTAGAATAAGA | hSINT1 | AGGTAGAATAAGAACC |
| #66 | ACC | #137 | CCA |
| | SEQ ID NO 305 | | SEQ ID NO 328 |
| hSINT1 | TTTTCGTGAGACGGAG | hSINT1 | AGTAAGGGAGGAGGAA |
| #67 | TCT | #138 | ACA |
| | SEQ ID NO 306 | | SEQ ID NO 329 |
| hSINT1 | TATTTCCCTGAACAGG | hSINT1 | GGGAGAGCCAGAAATA |
| #68 | TAG | #139 | GCTGGTTTA |
| | SEQ ID NO 307 | | SEQ ID NO 330 |
| hSINT1 | TTTTTTTGTTTGTTTG | hSINT1 | CCTCCTGGGATTACCA |
| #69 | TTG | #140 | TGAACCAAT |
| | SEQ ID NO 308 | | SEQ ID NO 331 |
| hSINT1 | ATTCTCCTGCCTCAGC | hSINT1 | CCTGGGATTACCATGA |
| #70 | TCC | #141 | ACCAATGCA |
| | SEQ ID NO 309 | | SEQ ID NO 332 |
| hSINT1 | TATTTCTGGCTCTCCC | hSINT1 | GGGATTACCATGAACC |
| #71 | CGC | #142 | AATGCAGTA |
| | SEQ ID NO 333 | | SEQ ID NO 339 |
| hSINT1 | TTTGTTTGTTTTTCGT | hSINT1 | GGATTACCATGAACCA |
| #72 | GAG | #143 | ATGCAGTAA |
| | SEQ ID NO 334 | | SEQ ID NO 340 |
| hSINT1 | TTTAATTAACATTCGG | hSINT1 | GATTACCATGAACCAA |
| #73 | ATA | #144 | TGCAGTAAT |
| | SEQ ID NO 335 | | SEQ ID NO 341 |
| hSINT1 | TCATGGTAATCCCAGG | hSINT1 | CCTCCTCCCTTACTCA |
| #74 | AGG | #145 | CTCTCTCTA |
| | SEQ ID NO 336 | | SEQ ID NO 342 |
| hSINT1 | TTGTTTTTCGTGAGAC | hSINT1 | CCTGGAGGTGCTCATT |
| #75 | GGA | #146 | GAATTTACT |
| | SEQ ID NO 337 | | SEQ ID NO 343 |
| hSINT1 | AAATGTCATTTTCCTT | hSINT1 | TGGAGGTGCTCATTGA |
| #76 | GGG | #147 | ATTTACTTT |
| | SEQ ID NO 338 | | SEQ ID NO 344 |
| | | hSINT1 | AGTCTCTGTTACCAGA |
| | | #148 | GAATCAGAA |
| | | | SEQ ID NO 345 |

1. A method for the inhibition or treatment of heart disease, particularly cardiomyopathy, comprising administering to a patient in need thereof a therapeutically effective amount of an oligonucleic acid agent directed at and capable of specifically inhibiting and/or degrading the enhancer RNA SINT1 (SEQ ID NO 001).

2. The method of claim 1, wherein the oligonucleic acid agent comprises, or essentially consists of a hybridizing sequence of nucleotides, which is capable of forming a hybrid with the enhancer RNA SINT1 (SEQ ID NO 001).

3. The method of claim 1, wherein the oligonucleic acid agent comprises, or essentially consists of, a sequence selected from Table 3, particularly wherein the sequence is selected from SEQ ID NOs 005, 006, 060, 061, 062, 063, 064, 066, 67, 068 and 069.

4. The method of claim 1, wherein the oligonucleic acid agent is an antisense oligonucleotide.

5. The method of claim 1, wherein the oligonucleic acid agent comprises one or several, or essentially consists of, locked nucleic acid (LNA) moieties and/or peptide nucleic acid (PNA) moieties, particularly wherein the oligonucleic acid agent essentially consists of locked nucleic acid (LNA) moieties.

6. The method of claim 5, wherein the LNA moieties are connected by thiophosphate bonds.

7. The method of claim 1, wherein the oligonucleic acid agent comprises 12-20 nucleotides, particularly 14-16 nucleotides.

8. The method of claim 1, wherein the oligonucleic acid agent comprises or essentially consists of a central block of 5 to 10 deoxyribonucleotides flanked on either side by 2′-O modified ribonucleotides or PNA oligomers, more particularly a central block of 5 to 10 deoxyribonucleosides flanked by LNA nucleoside analogues, even more particularly wherein said LNA nucleoside analogues are linked by phosphothioate moieties.

9. The method of claim 1, wherein the oligonucleic acid agent comprises or essentially consists of a sequence selected from Table 4, particularly wherein the sequence is selected from SEQ ID NOs 008, 009, 203, 204, 205, 206, 207, 209, 210, 211 and 212, wherein
the capital letters signify nucleoside analogues, particularly LNA, more particularly LNA linked by phosphothioate esters, and
the lower case letters signify DNA nucleosides linked by phosphate esters,
and the link between a nucleoside analogue and a DNA nucleoside is selected from phosphate ester and thiophosphate.

10. The method of claim 1, wherein the oligonucleic acid agent is linked to a nanoparticle, or encapsulated in a virus or a lipid complex.

11. The method of claim 1, wherein the heart disease is selected from cardiomyopathy, hypertrophic cardiomyopathy, cardiomyopathy resulting of cardiac overload, aortic stenosis, hypertension, and heart failure.

12. The method of claim 1, wherein the heart disease is hypertension-induced pathologic hypertrophy, stenosis-induced pathologic hypertrophy, congenital hypertrophic cardiomyopathy, idiopathic hypertrophic cardiomyopathy, restrictive pathologic hypertrophy or ischemic heart disease.

13. (canceled)

14. An oligonucleotide agent directed against the enhancer RNA SINT1 (SEQ ID No 001) that comprises or consists of any one of the sequence of SEQ ID NO 060 to SEQ ID NO 202, in particular sequences of SEQ ID NO 060 to SEQ ID NO 069.

15. A method for the manufacture of a medicament for use in the method of claim 1 for the inhibition or treatment of heart disease, particularly of cardiomyopathy, hypertrophic cardiomyopathy, cardiomyopathy resulting of cardiac overload, aortic stenosis, hypertension, heart failure, hypertension-induced pathologic hypertrophy, stenosis-induced pathologic hypertrophy, congenital hypertrophic cardiomyopathy, idiopathic hypertrophic cardiomyopathy, restrictive pathologic hypertrophy or ischemic heart disease.


SEQUENCE LISTING (345 sequences)

SEQ ID NO: 1 - DNA, 1109 bp, Homo sapiens
aatttccttattttggaagtgagaaaacagagacccagcatagcagagtgacttcctctaagggatccaggtatcccagccctaacctctttccataccgcactgcccaggatacaaaagcatcctaaaactcctgatttgtcatgactgcacatatgcgcacatatgtcaaagcgagtgtgggtttggagactctagagaaatgaaccagccataaatcattttcaagctaaggaaaaaagacttaaatatatgaatgggattagtacaaaaggatcttgtcaattaaaaaattccaggacaggctgggcacggtggttcgcgcctgtaatcccagcactttgggaggccaaggcgggcggatcacaaggtcaggagtttgagaccagcctggccgatatggagaaaccctgtctctactaaaaatacaaaaattagctgggcgtggtggtgggcacctgtagtcccagctacttgggagctgaggcaggagaatccatcatgccactgcactccagtctgggcaaaagagggagactccgtctcacgaaaaacaaacaaacaaacaacaaacaaacaaaaaaaacccaaggaaaatgacatttctgttgcgtttggtcagaaggcccattgcaatctggggttcttattctacctgttcagggaaatacgaagtccagatgaatgtgtattccaattctgactcaggcttggtttcactttctaaattgctcagcaggaatctgataagatgctgagaaaagagtaaaaagaaaaaattggagggtggggcggggagagccagaaatagctggtttaggggaggcctcctgggattaccatgaaccaatgcagtaattaataatatgaggaacagaatctatccgaatgttaattaaagtgtttcctcctcccttactcactctctctagggccccctggaggtgctcattgaatttacttttcccaggccattctccgctcccctagacagatcatctgaggtggggtgcgggggttgggcgggaaaacagtctctgttaccagagaatcagaaaaacaaaagctggccgggctcggtggctcacgcctgtaatcccagcactttg

SEQ ID NO: 2 - DNA, 57 bp, Artificial Sequence
  shRNA
ccggggcacatgcgccacttaatcactcgagtgattaagtggcgcatgtgctttttg

SEQ ID NO: 3 - DNA, 58 bp, Artificial Sequence
  shRNA
ccggggtacattgacctgtacttccctcgagggaagtacaggtcaatgtacctttttg

SEQ ID NO: 4 - DNA, 16 bp, Artificial Sequence
  Gapmer
tcatactatatgacag

SEQ ID NO: 5 - DNA, 16 bp, Artificial Sequence
  Gapmer
tgcttgaaagtgatga

SEQ ID NO: 6 - DNA, 16 bp, Artificial Sequence
  Gapmer
gtagaaagtggctaga

SEQ ID NO: 7 - DNA, 15 bp, Artificial Sequence
  Gapmer
aacacgtctatacgc

SEQ ID NO: 8 - DNA, 16 bp, Artificial Sequence
  Gapmer
tgcttgaaagtgatga

SEQ ID NO: 9 - DNA, 16 bp, Artificial Sequence
  Gapmer
gtagaaagtggctaga

SEQ ID NO: 10 - DNA, 21 bp, Artificial Sequence
  Primer
ggggctgtactgcttaaccag

SEQ ID NO: 11 - DNA, 20 bp, Artificial Sequence
  Primer
tgctcatcagttgccacttc

SEQ ID NO: 12 - DNA, 20 bp, Artificial Sequence
  Primer
actgagacccgaggcagtaa

SEQ ID NO: 13 - DNA, 20 bp, Artificial Sequence
  Primer
gtcttcaagcctgccttcac

SEQ ID NO: 14 - DNA, 21 bp, Artificial Sequence
  Primer
agggtggctacagtgtcaatg

SEQ ID NO: 15 - DNA, 20 bp, Artificial Sequence
  Primer
cccaacgacttccgaccata

SEQ ID NO: 16 - DNA, 19 bp, Artificial Sequence
  Primer
acgctcgtcctcagacaca

SEQ ID NO: 17 - DNA, 21 bp, Artificial Sequence
  Primer
ctcaacgggtttgatgtcgat

SEQ ID NO: 18 - DNA, 20 bp, Artificial Sequence
  Primer
gtgcggtgtccaacacagat

SEQ ID NO: 19 - DNA, 22 bp, Artificial Sequence
  Primer
tccaatcctgtcaatcctaccc

SEQ ID NO: 20 - DNA, 20 bp, Artificial Sequence
  Primer
agtccttcggtctcaaggca

SEQ ID NO: 21 - DNA, 21 bp, Artificial Sequence
  Primer
ccgatccggtctatcttgtgc

SEQ ID NO: 22 - DNA, 21 bp, Artificial Sequence
  Primer
tgtttgtcctatttcggggtg

SEQ ID NO: 23 - DNA, 21 bp, Artificial Sequence
  Primer
aatccgcacaagcaggtcttc

SEQ ID NO: 24 - DNA, 21 bp, Artificial Sequence
  Primer
gagaacgctcacacaaagacc

SEQ ID NO: 25 - DNA, 19 bp, Artificial Sequence
  Primer
caattcgttggagccccat

SEQ ID NO: 26 - DNA, 21 bp, Artificial Sequence
  Primer
aaagtgcaatacctcactcgc

SEQ ID NO: 27 - DNA, 22 bp, Artificial Sequence
  Primer
ggcatttcaatagtggaggctc

SEQ ID NO: 28 - DNA, 21 bp, Artificial Sequence
  Primer
gacaccacaaggtgcaaagtc

SEQ ID NO: 29 - DNA, 22 bp, Artificial Sequence
  Primer
cccactgttcttgaccgtatct

SEQ ID NO: 30 - DNA, 21 bp, Artificial Sequence
  Primer
tgatcgcctgcttattcacgg

SEQ ID NO: 31 - DNA, 21 bp, Artificial Sequence
  Primer
aaccgcctagaaatctccaga

SEQ ID NO: 32 - DNA, 23 bp, Artificial Sequence
  Primer
agaaggagttgtcggatattgct

SEQ ID NO: 33 - DNA, 21 bp, Artificial Sequence
  Primer
ttctccaccccaatttggctc

SEQ ID NO: 34 - DNA, 22 bp, Artificial Sequence
  Primer
cagttcggctataacactggtg

SEQ ID NO: 35 - DNA, 18 bp, Artificial Sequence
  Primer
gcccccgacagagaagat

SEQ ID NO: 36 - DNA, 21 bp, Artificial Sequence
  Primer
aggtcggtgtgaacggatttg

SEQ ID NO: 37 - DNA, 23 bp, Artificial Sequence
  Primer
tgtagaccatgtagttgaggtca

SEQ ID NO: 38 - DNA, 20 bp, Artificial Sequence
  Primer
agggaggtcgagctgttctc

SEQ ID NO: 39 - DNA, 21 bp, Artificial Sequence
  Primer
ggagtgttcactaagcggtca

SEQ ID NO: 40 - DNA, 23 bp, Artificial Sequence
  Primer
aacatcgagtgtcgaatatgtgg

SEQ ID NO: 41 - DNA, 21 bp, Artificial Sequence
  Primer
agccgaatagttcgccgaaag

SEQ ID NO: 42 - DNA, 21 bp, Artificial Sequence
  Primer
ggaagaccactcgcattcctt

SEQ ID NO: 43 - DNA, 22 bp, Artificial Sequence
  Primer
gtaatcagcaaccattgggtca

SEQ ID NO: 44 - DNA, 21 bp, Artificial Sequence
  Primer
cctggcgtcgtgattagtgat

SEQ ID NO: 45 - DNA, 22 bp, Artificial Sequence
  Primer
agacgttcagtcctgtccataa

SEQ ID NO: 46 - DNA, 23 bp, Artificial Sequence
  Primer
ccagaagttaacaaagaaagagg

SEQ ID NO: 47 - DNA, 25 bp, Artificial Sequence
  Primer
cttccaaaataaggaaatttgatgc

SEQ ID NO: 48 - DNA, 20 bp, Artificial Sequence
  Primer
ggtggctcgatgttaccctc

SEQ ID NO: 49 - DNA, 19 bp, Artificial Sequence
  Primer
ctgcgtgagcgaaggtttc

SEQ ID NO: 50 - DNA, 19 bp, Artificial Sequence
  Primer
attccccggatggaagacg

SEQ ID NO: 51 - DNA, 23 bp, Artificial Sequence
  Primer
cgccaaactcgatgggtttaata

SEQ ID NO: 52 - DNA, 20 bp, Artificial Sequence
  Primer
ccaagccgggtaagaagacc

SEQ ID NO: 53 - DNA, 20 bp, Artificial Sequence
  Primer
gtcagcgtggacagctcata

SEQ ID NO: 54 - DNA, 20 bp, Artificial Sequence
  Primer
ccaccacgtactcctctgtt

SEQ ID NO: 55 - DNA, 20 bp, Artificial Sequence
  Primer
ggttgctgtcatgtccgaaa

SEQ ID NO: 56 - DNA, 22 bp, Artificial Sequence
  Primer
gatagcggccgcgctcgctttc

SEQ ID NO: 57 - DNA, 22 bp, Artificial Sequence
  Primer
gcatttgcaatgaaaataaatg

SEQ ID NO: 58 - DNA, 21 bp, Artificial Sequence
  Primer
ccacagagcaggaagcagaga

SEQ ID NO: 59 - DNA, 24 bp, Artificial Sequence
  Primer
ggtttgaatgcgaaatgtccttac

SEQ ID NO: 60 - DNA, 16 bp, Artificial Sequence
  Gapmer
agaatctatccgaatg

SEQ ID NO: 61 - DNA, 16 bp, Artificial Sequence
  Gapmer
ttaaagtgtttcctcc

SEQ ID NO: 62 - DNA, 16 bp, Artificial Sequence
  Gapmer
actttctaaattgctc

SEQ ID NO: 63 - DNA, 16 bp, Artificial Sequence
  Gapmer
atctatccgaatgtta

SEQ ID NO: 64 - DNA, 16 bp, Artificial Sequence
  Gapmer
tctaaattgctcagca

SEQ ID NO: 65 - DNA, 16 bp, Artificial Sequence
  Gapmer
tactcactctctctag

SEQ ID NO: 66 - DNA, 15 bp, Artificial Sequence
  Gapmer
tcccaggccattctc

SEQ ID NO: 67 - DNA, 15 bp, Artificial Sequence
  Gapmer
acagaatctatccga

SEQ ID NO: 68 - DNA, 16 bp, Artificial Sequence
  Gapmer
taattaaagtgtttcc

SEQ ID NO: 69 - DNA, 16 bp, Artificial Sequence
  Gapmer
tacttttcccaggcca

SEQ ID NO: 70 - DNA, 19 bp, Artificial Sequence
  Gapmer
ttagaaagtgaaaccaagc

SEQ ID NO: 71 - DNA, 19 bp, Artificial Sequence
  Gapmer
tttttactcttttctcagc

SEQ ID NO: 72 - DNA, 19 bp, Artificial Sequence
  Gapmer
ttaacattcggatagattc

SEQ ID NO: 73 - DNA, 19 bp, Artificial Sequence
  Gapmer
ttactcttttctcagcatc

SEQ ID NO: 74 - DNA, 19 bp, Artificial Sequence
  Gapmer
tcatattattaattactgc

SEQ ID NO: 75 - DNA, 19 bp, Artificial Sequence
  Gapmer
ttactgcattggttcatgg

SEQ ID NO: 76 - DNA, 19 bp, Artificial Sequence
  Gapmer
tttctcagcatcttatcag

SEQ ID NO: 77 - DNA, 19 bp, Artificial Sequence
  Gapmer
ttaattactgcattggttc

SEQ ID NO: 78 - DNA, 19 bp, Artificial Sequence
  Gapmer
ttattaattactgcattgg

SEQ ID NO: 79 - DNA, 19 bp, Artificial Sequence
  Gapmer
taaaccagctatttctggc

SEQ ID NO: 80 - DNA, 19 bp, Artificial Sequence
  Gapmer
tttagaaagtgaaaccaag

SEQ ID NO: 81 - DNA, 19 bp, Artificial Sequence
  Gapmer
aaaagtaaattcaatgagc

SEQ ID NO: 82 - DNA, 19 bp, Artificial Sequence
  Gapmer
ttctcagcatcttatcaga

SEQ ID NO: 83 - DNA, 19 bp, Artificial Sequence
  Gapmer
tatcagattcctgctgagc

SEQ ID NO: 84 - DNA, 19 bp, Artificial Sequence
  Gapmer
tttctgattctctggtaac

SEQ ID NO: 85 - DNA, 19 bp, Artificial Sequence
  Gapmer
ttttctttttactcttttc

SEQ ID NO: 86 - DNA, 19 bp, Artificial Sequence
  Gapmer
ttaattaacattcggatag

SEQ ID NO: 87 - DNA, 19 bp, Artificial Sequence
  Gapmer
taaattcaatgagcacctc

SEQ ID NO: 88 - DNA, 19 bp, Artificial Sequence
  Gapmer
taacattcggatagattct

SEQ ID NO: 89 - DNA, 19 bp, Artificial Sequence
  Gapmer
aacagaaatgtcattttcc

SEQ ID NO: 90 - DNA, 19 bp, Artificial Sequence
  Gapmer
taacagagactgttttccc

SEQ ID NO: 91 - DNA, 19 bp, Artificial Sequence
  Gapmer
tttgtttttcgtgagacgg

SEQ ID NO: 92 - DNA, 19 bp, Artificial Sequence
  Gapmer
ttttctcagcatcttatca

SEQ ID NO: 93 - DNA, 19 bp, Artificial Sequence
  Gapmer
attggaatacacattcatc

SEQ ID NO: 94 - DNA, 19 bp, Artificial Sequence
  Gapmer
ttgtttttctgattctctg

SEQ ID NO: 95 - DNA, 19 bp, Artificial Sequence
  Gapmer
tattaattactgcattggt

SEQ ID NO: 96 - DNA, 19 bp, Artificial Sequence
  Gapmer
ttatcagattcctgctgag

SEQ ID NO: 97 - DNA, 19 bp, Artificial Sequence
  Gapmer
aaattcaatgagcacctcc

SEQ ID NO: 98 - DNA, 19 bp, Artificial Sequence
  Gapmer
ttttgtttttctgattctc

SEQ ID NO: 99 - DNA, 19 bp, Artificial Sequence
  Gapmer
atctggacttcgtatttcc

SEQ ID NO: 100 - DNA, 19 bp, Artificial Sequence
  Gapmer
ttttactcttttctcagca

SEQ ID NO: 101 - DNA, 19 bp, Artificial Sequence
  Gapmer
atacacattcatctggact

SEQ ID NO: 102 - DNA, 19 bp, Artificial Sequence
  Gapmer
tttttcgtgagacggagtc

SEQ ID NO: 103 - DNA, 19 bp, Artificial Sequence
  Gapmer
aatacacattcatctggac

SEQ ID NO: 104 - DNA, 19 bp, Artificial Sequence
  Gapmer
attctctggtaacagagac

SEQ ID NO: 105 - DNA, 19 bp, Artificial Sequence
  Gapmer
tttgtttgtttgttgtttg

SEQ ID NO: 106 - DNA, 19 bp, Artificial Sequence
  Gapmer
tttgtttgttgtttgtttg

SEQ ID NO: 107 - DNA, 19 bp, Artificial Sequence
  Gapmer
taattaacattcggataga

SEQ ID NO: 108 - DNA, 19 bp, Artificial Sequence
  Gapmer
aagtaaattcaatgagcac

SEQ ID NO: 109 - DNA, 19 bp, Artificial Sequence
  Gapmer
tgtttttctgattctctgg

SEQ ID NO: 110 - DNA, 19 bp, Artificial Sequence
  Gapmer
ttctgattctctggtaaca

SEQ ID NO: 111 - DNA, 19 bp, Artificial Sequence
  Gapmer
attcggatagattctgttc

SEQ ID NO: 112 - DNA, 19 bp, Artificial Sequence
  Gapmer
tttactcttttctcagcat

SEQ ID NO: 113 - DNA, 19 bp, Artificial Sequence
  Gapmer
ttttttgtttgtttgttgt

SEQ ID NO: 114 - DNA, 19 bp, Artificial Sequence
  Gapmer
attattaattactgcattg

SEQ ID NO: 115 - DNA, 19 bp, Artificial Sequence
  Gapmer
tttgtttgtttgtttttcg

SEQ ID NO: 116 - DNA, 19 bp, Artificial Sequence
  Gapmer
taattactgcattggttca

SEQ ID NO: 117 - DNA, 19 bp, Artificial Sequence
  Gapmer
attggttcatggtaatccc

SEQ ID NO: 118 - DNA, 19 bp, Artificial Sequence
  Gapmer
tcgtatttccctgaacagg

SEQ ID NO: 119 - DNA, 19 bp, Artificial Sequence
  Gapmer
attactgcattggttcatg

SEQ ID NO: 120 - DNA, 19 bp, Artificial Sequence
  Gapmer
aacaggtagaataagaacc

SEQ ID NO: 121 - DNA, 19 bp, Artificial Sequence
  Gapmer
ttttcgtgagacggagtct

SEQ ID NO: 122 - DNA, 19 bp, Artificial Sequence
  Gapmer
tatttccctgaacaggtag

SEQ ID NO: 123 - DNA, 19 bp, Artificial Sequence
  Gapmer
tttttttgtttgtttgttg

SEQ ID NO: 124 - DNA, 19 bp, Artificial Sequence
  Gapmer
attctcctgcctcagctcc

SEQ ID NO: 125 - DNA, 19 bp, Artificial Sequence
  Gapmer
tatttctggctctccccgc

SEQ ID NO: 126 - DNA, 19 bp, Artificial Sequence
  Gapmer
tttgtttgtttttcgtgag

SEQ ID NO: 127 - DNA, 19 bp, Artificial Sequence
  Gapmer
tttaattaacattcggata

SEQ ID NO: 128 - DNA, 19 bp, Artificial Sequence
  Gapmer
tcatggtaatcccaggagg

SEQ ID NO: 129 - DNA, 19 bp, Artificial Sequence
  Gapmer
ttgtttttcgtgagacgga

SEQ ID NO: 130 - DNA, 19 bp, Artificial Sequence
  Gapmer
aaatgtcattttccttggg

SEQ ID NO: 131 - DNA, 19 bp, Artificial Sequence
  Gapmer
atcttatcagattcctgct

SEQ ID NO: 132 - DNA, 19 bp, Artificial Sequence
  Gapmer
aacattcggatagattctg

SEQ ID NO: 133 - DNA, 19 bp, Artificial Sequence
  Gapmer
tcttatcagattcctgctg

SEQ ID NO: 134 - DNA, 19 bp, Artificial Sequence
  Gapmer
agtaaattcaatgagcacc

SEQ ID NO: 135 - DNA, 19 bp, Artificial Sequence
  Gapmer
aaacgcaacagaaatgtca

SEQ ID NO: 136 - DNA, 19 bp, Artificial Sequence
  Gapmer
taagggaggaggaaacact

SEQ ID NO: 137 - DNA, 19 bp, Artificial Sequence
  Gapmer
ttgtttgtttttcgtgaga

SEQ ID NO: 138 - DNA, 19 bp, Artificial Sequence
  Gapmer
tgtttgtttttcgtgagac

SEQ ID NO: 139 - DNA, 19 bp, Artificial Sequence
  Gapmer
aattttttctttttactct

SEQ ID NO: 140 - DNA, 19 bp, Artificial Sequence
  Gapmer
attcaatgagcacctccag

SEQ ID NO: 141 - DNA, 19 bp, Artificial Sequence
  Gapmer
aaccagctatttctggctc

SEQ ID NO: 142 - DNA, 19 bp, Artificial Sequence
  Gapmer
aataagaaccccagattgc

SEQ ID NO: 143 - DNA, 19 bp, Artificial Sequence
  Gapmer
tttttctgattctctggta

SEQ ID NO: 144 - DNA, 19 bp, Artificial Sequence
  Gapmer
caatttagaaagtgaaacc

SEQ ID NO: 145 - DNA, 19 bp, Artificial Sequence
  Gapmer
acactttaattaacattcg

SEQ ID NO: 146 - DNA, 19 bp, Artificial Sequence
  Gapmer
acacattcatctggacttc

SEQ ID NO: 147 - DNA, 19 bp, Artificial Sequence
  Gapmer
tgtttttcgtgagacggag

SEQ ID NO: 148 - DNA, 19 bp, Artificial Sequence
  Gapmer
tgtttgtttgtttttcgtg

SEQ ID NO: 149 - DNA, 19 bp, Artificial Sequence
  Gapmer
aaagtaaattcaatgagca

SEQ ID NO: 150 - DNA, 19 bp, Artificial Sequence
  Gapmer
agtcagaattggaatacac

SEQ ID NO: 151 - DNA, 19 bp, Artificial Sequence
  Gapmer
cactttaattaacattcgg

SEQ ID NO: 152 - DNA, 19 bp, Artificial Sequence
  Gapmer
aggaaacactttaattaac

SEQ ID NO: 153 - DNA, 19 bp, Artificial Sequence
  Gapmer
aatgtcattttccttgggt

SEQ ID NO: 154 - DNA, 19 bp, Artificial Sequence
  Gapmer
agaattggaatacacattc

SEQ ID NO: 155 - DNA, 19 bp, Artificial Sequence
  Gapmer
aattaacattcggatagat

SEQ ID NO: 156 - DNA, 19 bp, Artificial Sequence
  Gapmer
aattggaatacacattcat

SEQ ID NO: 157 - DNA, 19 bp, Artificial Sequence
  Gapmer
aagtgaaaccaagcctgag

SEQ ID NO: 158 - DNA, 19 bp, Artificial Sequence
  Gapmer
aatttagaaagtgaaacca

SEQ ID NO: 159 - DNA, 19 bp, Artificial Sequence
  Gapmer
atagattctgttcctcata

SEQ ID NO: 160 - DNA, 19 bp, Artificial Sequence
  Gapmer
aaagtgaaaccaagcctga

SEQ ID NO: 161 - DNA, 19 bp, Artificial Sequence
  Gapmer
caacagaaatgtcattttc

SEQ ID NO: 162 - DNA, 19 bp, Artificial Sequence
  Gapmer
cttttctcagcatcttatc

SEQ ID NO: 163 - DNA, 19 bp, Artificial Sequence
  Gapmer
atttccctgaacaggtaga

SEQ ID NO: 164 - DNA, 19 bp, Artificial Sequence
  Gapmer
agaaatgtcattttccttg

SEQ ID NO: 165 - DNA, 19 bp, Artificial Sequence
  Gapmer
aacagagactgttttcccg

SEQ ID NO: 166 - DNA, 19 bp, Artificial Sequence
  Gapmer
aaaccaagcctgagtcaga

SEQ ID NO: 167 - DNA, 19 bp, Artificial Sequence
  Gapmer
ctcagatgatctgtctagg

SEQ ID NO: 168 - DNA, 19 bp, Artificial Sequence
  Gapmer
aattcaatgagcacctcca

SEQ ID NO: 169 - DNA, 19 bp, Artificial Sequence
  Gapmer
atcagattcctgctgagca

SEQ ID NO: 170 - DNA, 19 bp, Artificial Sequence
  Gapmer
atgatctgtctaggggagc

SEQ ID NO: 171 - DNA, 19 bp, Artificial Sequence
  Gapmer
aattactgcattggttcat

SEQ ID NO: 172 - DNA, 19 bp, Artificial Sequence
  Gapmer
atgatggattctcctgcct

SEQ ID NO: 173 - DNA, 19 bp, Artificial Sequence
  Gapmer
acttcgtatttccctgaac

SEQ ID NO: 174 - DNA, 19 bp, Artificial Sequence
  Gapmer
accaaacgcaacagaaatg

SEQ ID NO: 175 - DNA, 19 bp, Artificial Sequence
  Gapmer
actttaattaacattcgga

SEQ ID NO: 176 - DNA, 19 bp, Artificial Sequence
  Gapmer
agtgaaaccaagcctgagt

SEQ ID NO: 177 - DNA, 19 bp, Artificial Sequence
  Gapmer
attgcaatgggccttctga

SEQ ID NO: 178 - DNA, 19 bp, Artificial Sequence
  Gapmer
gaaatgtcattttccttgg

SEQ ID NO: 179 - DNA, 19 bp, Artificial Sequence
  Gapmer
aatgagcacctccaggggg

SEQ ID NO: 180 - DNA, 19 bp, Artificial Sequence
  Gapmer
ctagagagagtgagtaagg

SEQ ID NO: 181 - DNA, 19 bp, Artificial Sequence
  Gapmer
ataagaaccccagattgca

SEQ ID NO: 182 - DNA, 19 bp, Artificial Sequence
  Gapmer
agattctgttcctcatatt

SEQ ID NO: 183 - DNA, 19 bp, Artificial Sequence
  Gapmer
actgttttcccgcccaacc

SEQ ID NO: 184 - DNA, 19 bp, Artificial Sequence
  Gapmer
aagaaccccagattgcaat

SEQ ID NO: 185 - DNA, 19 bp, Artificial Sequence
  Gapmer
agagagtgagtaagggagg

SEQ ID NO: 186 - DNA, 19 bp, Artificial Sequence
  Gapmer
agaaccccagattgcaatg

SEQ ID NO: 187 - DNA, 19 bp, Artificial Sequence
  Gapmer
agagactgttttcccgccc

SEQ ID NO: 188 - DNA, 19 bp, Artificial Sequence
  Gapmer
agatgatctgtctagggga

SEQ ID NO: 189 - DNA, 19 bp, Artificial Sequence
  Gapmer
agaatggcctgggaaaagt

SEQ ID NO: 190 - DNA, 19 bp, Artificial Sequence
  Gapmer
agagagagtgagtaaggga

SEQ ID NO: 191 - DNA, 19 bp, Artificial Sequence
  Gapmer
aggtagaataagaacccca

SEQ ID NO: 192 - DNA, 19 bp, Artificial Sequence
  Gapmer
agtaagggaggaggaaaca

SEQ ID NO: 193 - DNA, 25 bp, Artificial Sequence
  Gapmer
gggagagccagaaatagctggttta

SEQ ID NO: 194 - DNA, 25 bp, Artificial Sequence
  Gapmer
cctcctgggattaccatgaaccaat

SEQ ID NO: 195 - DNA, 25 bp, Artificial Sequence
  Gapmer
cctgggattaccatgaaccaatgca

SEQ ID NO: 196 - DNA, 25 bp, Artificial Sequence
  Gapmer
gggattaccatgaaccaatgcagta

SEQ ID NO: 197 - DNA, 25 bp, Artificial Sequence
  Gapmer
ggattaccatgaaccaatgcagtaa

SEQ ID NO: 198 - DNA, 25 bp, Artificial Sequence
  Gapmer
gattaccatgaaccaatgcagtaat

SEQ ID NO: 199 - DNA, 25 bp, Artificial Sequence
  Gapmer
cctcctcccttactcactctctcta

SEQ ID NO: 200 - DNA, 25 bp, Artificial Sequence
  Gapmer
cctggaggtgctcattgaatttact

SEQ ID NO: 201 - DNA, 25 bp, Artificial Sequence
  Gapmer
tggaggtgctcattgaatttacttt

SEQ ID NO: 202 - DNA, 25 bp, Artificial Sequence
  Gapmer
agtctctgttaccagagaatcagaa

SEQ ID NO: 203 - DNA, 16 bp, Artificial Sequence
  Gapmer
agaatctatccgaatg

SEQ ID NO: 204 - DNA, 16 bp, Artificial Sequence
  Gapmer
ttaaagtgtttcctcc

SEQ ID NO: 205 - DNA, 16 bp, Artificial Sequence
  Gapmer
actttctaaattgctc

SEQ ID NO: 206 - DNA, 16 bp, Artificial Sequence
  Gapmer
atctatccgaatgtta

SEQ ID NO: 207 - DNA, 16 bp, Artificial Sequence
  Gapmer
tctaaattgctcagca

SEQ ID NO: 208 - DNA, 16 bp, Artificial Sequence
  Gapmer
tactcactctctctag

SEQ ID NO: 209 - DNA, 15 bp, Artificial Sequence
  Gapmer
tcccaggccattctc

SEQ ID NO: 210 - DNA, 15 bp, Artificial Sequence
  Gapmer
acagaatctatccga

SEQ ID NO: 211 - DNA, 16 bp, Artificial Sequence
  Gapmer
taattaaagtgtttcc

SEQ ID NO: 212 - DNA, 16 bp, Artificial Sequence
  Gapmer
tacttttcccaggcca

SEQ ID NO: 213 - DNA, 19 bp, Artificial Sequence
  Gapmer
ttagaaagtgaaaccaagc

SEQ ID NO: 214 - DNA, 19 bp, Artificial Sequence
  Gapmer
tttttactcttttctcagc

SEQ ID NO: 215 - DNA, 19 bp, Artificial Sequence
  Gapmer
ttaacattcggatagattc

SEQ ID NO: 216 - DNA, 19 bp, Artificial Sequence
  Gapmer
ttactcttttctcagcatc

SEQ ID NO: 217 - DNA, 19 bp, Artificial Sequence
  Gapmer
tcatattattaattactgc

SEQ ID NO: 218 - DNA, 19 bp, Artificial Sequence
  Gapmer
ttactgcattggttcatgg

SEQ ID NO: 219 - DNA, 19 bp, Artificial Sequence
  Gapmer
tttctcagcatcttatcag

SEQ ID NO: 220 - DNA, 19 bp, Artificial Sequence
  Gapmer
ttaattactgcattggttc

SEQ ID NO: 221 - DNA, 19 bp, Artificial Sequence
  Gapmer
ttattaattactgcattgg

SEQ ID NO: 222 - DNA, 19 bp, Artificial Sequence
  Gapmer
atcttatcagattcctgct

SEQ ID NO: 223 - DNA, 19 bp, Artificial Sequence
  Gapmer
aacattcggatagattctg

SEQ ID NO: 224 - DNA, 19 bp, Artificial Sequence
  Gapmer
tcttatcagattcctgctg

SEQ ID NO: 225 - DNA, 19 bp, Artificial Sequence
  Gapmer
agtaaattcaatgagcacc

SEQ ID NO: 226 - DNA, 19 bp, Artificial Sequence
  Gapmer
aaacgcaacagaaatgtca

SEQ ID NO: 227 - DNA, 19 bp, Artificial Sequence
  Gapmer
taagggaggaggaaacact

SEQ ID NO: 228 - DNA, 19 bp, Artificial Sequence
  Gapmer
ttgtttgtttttcgtgaga

SEQ ID NO: 229 - DNA, 19 bp, Artificial Sequence
  Gapmer
tgtttgtttttcgtgagac

SEQ ID NO: 230 - DNA, 19 bp, Artificial Sequence
  Gapmer
aattttttctttttactct

SEQ ID NO: 231 - DNA, 19 bp, Artificial Sequence
  Gapmer
attcaatgagcacctccag

SEQ ID NO: 232 - DNA, 19 bp, Artificial Sequence
  Gapmer
aaccagctatttctggctc

SEQ ID NO: 233 - DNA, 19 bp, Artificial Sequence
  Gapmer
aataagaaccccagattgc

SEQ ID NO: 234 - DNA, 19 bp, Artificial Sequence
  Gapmer
tttttctgattctctggta

SEQ ID NO: 235 - DNA, 19 bp, Artificial Sequence
  Gapmer
caatttagaaagtgaaacc

SEQ ID NO: 236 - DNA, 19 bp, Artificial Sequence
  Gapmer
acactttaattaacattcg

SEQ ID NO: 237 - DNA, 19 bp, Artificial Sequence
  Gapmer
acacattcatctggacttc

SEQ ID NO: 238 - DNA, 19 bp, Artificial Sequence
  Gapmer
tgtttttcgtgagacggag

SEQ ID NO: 239 - DNA, 19 bp, Artificial Sequence
  Gapmer
tgtttgtttgtttttcgtg

SEQ ID NO: 240 - DNA, 19 bp, Artificial Sequence
  Gapmer
aaagtaaattcaatgagca

SEQ ID NO: 241 - DNA, 19 bp, Artificial Sequence
  Gapmer
taaaccagctatttctggc

SEQ ID NO: 242 - DNA, 19 bp, Artificial Sequence
  Gapmer
tttagaaagtgaaaccaag

SEQ ID NO: 243 - DNA, 19 bp, Artificial Sequence
  Gapmer
aaaagtaaattcaatgagc

SEQ ID NO: 244 - DNA, 19 bp, Artificial Sequence
  Gapmer
ttctcagcatcttatcaga

SEQ ID NO: 245 - DNA, 19 bp, Artificial Sequence
  Gapmer
tatcagattcctgctgagc

SEQ ID NO: 246 - DNA, 19 bp, Artificial Sequence
  Gapmer
tttctgattctctggtaac

SEQ ID NO: 247 - DNA, 19 bp, Artificial Sequence
  Gapmer
ttttctttttactcttttc

SEQ ID NO: 248 - DNA, 19 bp, Artificial Sequence
  Gapmer
ttaattaacattcggatag

SEQ ID NO: 249 - DNA, 19 bp, Artificial Sequence
  Gapmer
taaattcaatgagcacctc

SEQ ID NO: 250 - DNA, 19 bp, Artificial Sequence
  Gapmer
taacattcggatagattct

SEQ ID NO: 251 - DNA, 19 bp, Artificial Sequence
  Gapmer
aacagaaatgtcattttcc

SEQ ID NO: 252 - DNA, 19 bp, Artificial Sequence
  Gapmer
taacagagactgttttccc

SEQ ID NO: 253 - DNA, 19 bp, Artificial Sequence
  Gapmer
tttgtttttcgtgagacgg

SEQ ID NO: 254 - DNA, 19 bp, Artificial Sequence
  Gapmer
ttttctcagcatcttatca

SEQ ID NO: 255 - DNA, 19 bp, Artificial Sequence
  Gapmer
attggaatacacattcatc

SEQ ID NO: 256 - DNA, 19 bp, Artificial Sequence
  Gapmer
ttgtttttctgattctctg

SEQ ID NO: 257 - DNA, 19 bp, Artificial Sequence
  Gapmer
tattaattactgcattggt

SEQ ID NO: 258 - DNA, 19 bp, Artificial Sequence
  Gapmer
ttatcagattcctgctgag

SEQ ID NO: 259 - DNA, 19 bp, Artificial Sequence
  Gapmer
aaattcaatgagcacctcc

SEQ ID NO: 260 - DNA, 19 bp, Artificial Sequence
  Gapmer
ttttgtttttctgattctc

SEQ ID NO: 261 - DNA, 19 bp, Artificial Sequence
  Gapmer
atctggacttcgtatttcc

SEQ ID NO: 262 - DNA, 19 bp, Artificial Sequence
  Gapmer
ttttactcttttctcagca

SEQ ID NO: 263 - DNA, 19 bp, Artificial Sequence
  Gapmer
atacacattcatctggact

SEQ ID NO: 264 - DNA, 19 bp, Artificial Sequence
  Gapmer
agtcagaattggaatacac

SEQ ID NO: 265 - DNA, 19 bp, Artificial Sequence
  Gapmer
cactttaattaacattcgg

SEQ ID NO: 266 - DNA, 19 bp, Artificial Sequence
  Gapmer
aggaaacactttaattaac

SEQ ID NO: 267 - DNA, 19 bp, Artificial Sequence
  Gapmer
aatgtcattttccttgggt

SEQ ID NO: 268 - DNA, 19 bp, Artificial Sequence
  Gapmer
agaattggaatacacattc

SEQ ID NO: 269 - DNA, 19 bp, Artificial Sequence
  Gapmer
aattaacattcggatagat

SEQ ID NO: 270 - DNA, 19 bp, Artificial Sequence
  Gapmer
aattggaatacacattcat

SEQ ID NO: 271 - DNA, 19 bp, Artificial Sequence
  Gapmer
aagtgaaaccaagcctgag

SEQ ID NO: 272 - DNA, 19 bp, Artificial Sequence
  Gapmer
aatttagaaagtgaaacca

SEQ ID NO: 273 - DNA, 19 bp, Artificial Sequence
  Gapmer
atagattctgttcctcata

SEQ ID NO: 274 - DNA, 19 bp, Artificial Sequence
  Gapmer
aaagtgaaaccaagcctga

SEQ ID NO: 275 - DNA, 19 bp, Artificial Sequence
  Gapmer
caacagaaatgtcattttc

SEQ ID NO: 276 - DNA, 19 bp, Artificial Sequence
  Gapmer
cttttctcagcatcttatc

SEQ ID NO: 277 - DNA, 19 bp, Artificial Sequence
  Gapmer
atttccctgaacaggtaga

SEQ ID NO: 278 - DNA, 19 bp, Artificial Sequence
  Gapmer
agaaatgtcattttccttg

SEQ ID NO: 279 - DNA, 19 bp, Artificial Sequence
  Gapmer
aacagagactgttttcccg

SEQ ID NO: 280 - DNA, 19 bp, Artificial Sequence
  Gapmer
aaaccaagcctgagtcaga

SEQ ID NO: 281 - DNA, 19 bp, Artificial Sequence
  Gapmer
ctcagatgatctgtctagg

SEQ ID NO: 282 - DNA, 19 bp, Artificial Sequence
  Gapmer
aattcaatgagcacctcca

SEQ ID NO: 283 - DNA, 19 bp, Artificial Sequence
  Gapmer
atcagattcctgctgagca

SEQ ID NO: 284 - DNA, 19 bp, Artificial Sequence
  Gapmer
atgatctgtctaggggagc

SEQ ID NO: 285 - DNA, 19 bp, Artificial Sequence
  Gapmer
aattactgcattggttcat

SEQ ID NO: 286 - DNA, 19 bp, Artificial Sequence
  Gapmer
atgatggattctcctgcct

SEQ ID NO: 287 - DNA, 19 bp, Artificial Sequence
  Gapmer
tttttcgtgagacggagtc

SEQ ID NO: 288 - DNA, 19 bp, Artificial Sequence
  Gapmer
aatacacattcatctggac

SEQ ID NO: 289 - DNA, 19 bp, Artificial Sequence
  Gapmer
attctctggtaacagagac

SEQ ID NO: 290 - DNA, 19 bp, Artificial Sequence
  Gapmer
tttgtttgtttgttgtttg

SEQ ID NO: 291 - DNA, 19 bp, Artificial Sequence
  Gapmer
tttgtttgttgtttgtttg

SEQ ID NO: 292 - DNA, 19 bp, Artificial Sequence
  Gapmer
taattaacattcggataga

SEQ ID NO: 293 - DNA, 19 bp, Artificial Sequence
  Gapmer
aagtaaattcaatgagcac

SEQ ID NO: 294 - DNA, 19 bp, Artificial Sequence
  Gapmer
tgtttttctgattctctgg

SEQ ID NO: 295 - DNA, 19 bp, Artificial Sequence
  Gapmer
ttctgattctctggtaaca

SEQ ID NO: 296 - DNA, 19 bp, Artificial Sequence
  Gapmer
attcggatagattctgttc

SEQ ID NO: 297 - DNA, 19 bp, Artificial Sequence
  Gapmer
tttactcttttctcagcat

SEQ ID NO: 298 - DNA, 19 bp, Artificial Sequence
  Gapmer
ttttttgtttgtttgttgt

SEQ ID NO: 299 - DNA, 19 bp, Artificial Sequence
  Gapmer
attattaattactgcattg

SEQ ID NO: 300 - DNA, 19 bp, Artificial Sequence
  Gapmer
tttgtttgtttgtttttcg

SEQ ID NO: 301 - DNA, 19 bp, Artificial Sequence
  Gapmer
taattactgcattggttca

SEQ ID NO: 302 - DNA, 19 bp, Artificial Sequence
  Gapmer
attggttcatggtaatccc

SEQ ID NO: 303 - DNA, 19 bp, Artificial Sequence
  Gapmer
tcgtatttccctgaacagg

SEQ ID NO: 304 - DNA, 19 bp, Artificial Sequence
  Gapmer
attactgcattggttcatg

SEQ ID NO: 305 - DNA, 19 bp, Artificial Sequence
  Gapmer
aacaggtagaataagaacc

SEQ ID NO: 306 - DNA, 19 bp, Artificial Sequence
  Gapmer
ttttcgtgagacggagtct

SEQ ID NO: 307 - DNA, 19 bp, Artificial Sequence
  Gapmer
tatttccctgaacaggtag

SEQ ID NO: 308 - DNA, 19 bp, Artificial Sequence
  Gapmer
tttttttgtttgtttgttg

SEQ ID NO: 309 - DNA, 19 bp, Artificial Sequence
  Gapmer
attctcctgcctcagctcc

SEQ ID NO: 310 - DNA, 19 bp, Artificial Sequence
  Gapmer
acttcgtatttccctgaac

SEQ ID NO: 311 - DNA, 19 bp, Artificial Sequence
  Gapmer
accaaacgcaacagaaatg

SEQ ID NO: 312 - DNA, 19 bp, Artificial Sequence
  Gapmer
actttaattaacattcgga

SEQ ID NO: 313 - DNA, 19 bp, Artificial Sequence
  Gapmer
agtgaaaccaagcctgagt

SEQ ID NO: 314 - DNA, 19 bp, Artificial Sequence
  Gapmer
attgcaatgggccttctga

SEQ ID NO: 315 - DNA, 19 bp, Artificial Sequence
  Gapmer
gaaatgtcattttccttgg

SEQ ID NO: 316 - DNA, 19 bp, Artificial Sequence
  Gapmer
aatgagcacctccaggggg

SEQ ID NO: 317 - DNA, 19 bp, Artificial Sequence
  Gapmer
ctagagagagtgagtaagg

SEQ ID NO: 318 - DNA, 19 bp, Artificial Sequence
  Gapmer
ataagaaccccagattgca

SEQ ID NO: 319 - DNA, 19 bp, Artificial Sequence
  Gapmer
agattctgttcctcatatt

SEQ ID NO: 320 - DNA, 19 bp, Artificial Sequence
  Gapmer
actgttttcccgcccaacc

SEQ ID NO: 321 - DNA, 19 bp, Artificial Sequence
  Gapmer
aagaaccccagattgcaat

SEQ ID NO: 322 - DNA, 19 bp, Artificial Sequence
  Gapmer
agagagtgagtaagggagg

SEQ ID NO: 323 - DNA, 19 bp, Artificial Sequence
  Gapmer
agaaccccagattgcaatg

SEQ ID NO: 324 - DNA, 19 bp, Artificial Sequence
  Gapmer
agagactgttttcccgccc

SEQ ID NO: 325 - DNA, 19 bp, Artificial Sequence
  Gapmer
agatgatctgtctagggga

SEQ ID NO: 326 - DNA, 19 bp, Artificial Sequence
  Gapmer
agaatggcctgggaaaagt

SEQ ID NO: 327 - DNA, 19 bp, Artificial Sequence
  Gapmer
agagagagtgagtaaggga

SEQ ID NO: 328 - DNA, 19 bp, Artificial Sequence
  Gapmer
aggtagaataagaacccca

SEQ ID NO: 329 - DNA, 19 bp, Artificial Sequence
  Gapmer
agtaagggaggaggaaaca

SEQ ID NO: 330 - DNA, 25 bp, Artificial Sequence
  Gapmer
gggagagccagaaatagctggttta

SEQ ID NO: 331 - DNA, 25 bp, Artificial Sequence
  Gapmer
cctcctgggattaccatgaaccaat

SEQ ID NO: 332 - DNA, 25 bp, Artificial Sequence
  Gapmer
cctgggattaccatgaaccaatgca

SEQ ID NO: 333 - DNA, 19 bp, Artificial Sequence
  Gapmer
tatttctggctctccccgc

SEQ ID NO: 334 - DNA, 19 bp, Artificial Sequence
  Gapmer
tttgtttgtttttcgtgag

SEQ ID NO: 335 - DNA, 19 bp, Artificial Sequence
  Gapmer
tttaattaacattcggata

SEQ ID NO: 336 - DNA, 19 bp, Artificial Sequence
  Gapmer
tcatggtaatcccaggagg

SEQ ID NO: 337 - DNA, 19 bp, Artificial Sequence
  Gapmer
ttgtttttcgtgagacgga

SEQ ID NO: 338 - DNA, 19 bp, Artificial Sequence
  Gapmer
aaatgtcattttccttggg

SEQ ID NO: 339 - DNA, 25 bp, Artificial Sequence
  Gapmer
gggattaccatgaaccaatgcagta

SEQ ID NO: 340 - DNA, 25 bp, Artificial Sequence
  Gapmer
ggattaccatgaaccaatgcagtaa

SEQ ID NO: 341 - DNA, 25 bp, Artificial Sequence
  Gapmer
gattaccatgaaccaatgcagtaat

SEQ ID NO: 342 - DNA, 25 bp, Artificial Sequence
  Gapmer
cctcctcccttactcactctctcta

SEQ ID NO: 343 - DNA, 25 bp, Artificial Sequence
  Gapmer
cctggaggtgctcattgaatttact

SEQ ID NO: 344 - DNA, 25 bp, Artificial Sequence
  Gapmer
tggaggtgctcattgaatttacttt

SEQ ID NO: 345 - DNA, 25 bp, Artificial Sequence
  Gapmer
agtctctgttaccagagaatcagaa